(19) United States
(12) Patent Application Publication
(10) Pub. No.: US 2018/0320217 A1
(43) Pub. Date: Nov. 8, 2018

(54) METHODS FOR SAMPLING AND MEASURING ORAL LAVAGE PROTEINS

(71) Applicant: The Procter & Gamble Company, Cincinnati, OH (US)

(72) Inventors: John Christian HAUGHT, West Chester, OH (US); Sancai XIE, Liberty Township, OH (US); Cheryl Sue TANSKY, Forest Park, OH (US)

(21) Appl. No.: 15/971,719

(22) Filed: May 4, 2018

Related U.S. Application Data
(60) Provisional application No. 62/501,523, filed on May 4, 2017.

(51) Int. Cl. C12Q 1/32; C12Q 1/00

(52) U.S. Cl. CPC C12Q 1/32; G01N 2800/18; C12Q 2326/90; C12Q 1/008

(57) ABSTRACT

A method for reducing a tetrazolium salt.

### FIELD OF THE INVENTION

The invention is related to methods of collecting oral cavity samples, such as oral lavage, and extracting and analyzing proteins to monitor the health status of oral epithelium.

### BACKGROUND OF THE INVENTION

Periodontal diseases, such as gingivitis and periodontitis, involve chronic inflammation in the gingival tissue caused by microbial communities and host immune responses. They are one of the most ubiquitous diseases worldwide affecting up to 90% of the population, and remain the most common cause of tooth loss in the world today. In healthy gingiva, the microbial community is in a homeostatic equilibrium with the host, and host immune systems limit bacterial overgrowth and neutralize toxic products, such as lipopolysaccharides (LPS) and lipoteichoic acids (LTA). The intricate balance between host and bacteria is disrupted as bacteria overgrow in the gingival margins or in the subgingival crevice. Recent data from metagenomics studies showed that bacterial species were increased in gingivitis in supragingival and subgingival plaques, such as Prevotella pallens, Prevotella intermedia, Porphyromonas gingivalis, and Filifactor alocis. Although the etiology of gingivitis and periodontitis remains elusive, one thing is clear; the composition of the dental plaques is significantly different in healthy sites compared with clinically defined disease sites. This observation, together with advances in characterizing the host and bacterial interactions using the newly developed tools in genomics, proteomics and metabonomics, has led to the notion that gingivitis and periodontitis are the result of disrupted homeostasis between host and polymicrobial communities (Lamont R J and Hajishengallis G. Polymicrobial synergy and dysbiosis in inflammatory disease. G Trends Mol Med. 2015; 21:172-83).

Polymicrobial communities in the dental plaques produce various virulence factors; for example, many bacteria produce digestive enzymes, such as hyaluronidases to breakdown polysaccharides that glue the host cells together, fibrinolytic enzymes that lyse the fibrins of blood clots, and collagenases that degrade collagens in the connective tissues. Gram negative bacteria secrete endotoxins, also called lipopolysaccharide (LPS), lipids, and lipooligosaccharides, while Gram positive bacteria produce lipoteichoic acid (LTA) and peptiglycans. Furthermore, one pathogen bacterium can generate multiple virulence factors; for example P. gingivalis has been reported to generate multiple virulence factors that are involved in the inflammatory and destructive events of periodontal tissues. These virulence factors include the capsule, outer membrane, its associated LPS, fimbriae, proteinases, and selected enzymes.

Microbial virulence factors have been shown to act as inflammatory mediators by activating Toll-like receptors. Binding of LPS to TLR4, and LTA to TLR2, activates the NF-κB signaling pathway in immune cells and gingival epithelial cells, subsequently leading to production and release of proinflammatory cytokines and chemokines, such as IL-lα, IL-1β, IL-6, IL-8, IFN y, and TNF-α. Those microbial virulence factors also bring about profound changes in cellular metabolism, especially in production of Adenosine triphosphate (ATP).

Glucose is the major nutrient for adenosine triphosphate (ATP) production in our diet. There are three well-characterized pathways for extracting energy from glucose: glycolysis, cellular respiration and fermentation.

Glycolysis usually occurs in cytoplasm, and includes a glucose molecule being metabolized to produce 2 molecules of pyruvate, 2 molecules of ATP and 2 molecules of NADH+H⁺. Ten enzymes are involved in the glycolysis process, including hexokinase, phosphoglucose isomerase, phosphofructokinase, aldolase, triosephosphate isomerase, glyceraldehyde phosphate dehydrogenase, phosphoglycerate kinase, phosphoglycerate mutase, enolase, and pyruvate dehydrogenase.

Cellular respiration is a set of metabolic reactions to convert biochemical energy extracted from nutrients into (ATP), carbon dioxide and water. This process includes three sub-pathways—pyruvate oxidation, the citric acid cycle and the electron transport chain. The citric acid cycle—also known as the tricarboxylic acid cycle (TCA cycle) and the Krebs cycle—is a series of enzyme-catalyzed catabolic reactions, breaking a six carbon molecule into a four carbon molecule and two molecules of carbon dioxides. The chemical reactions occur in the matrix of the mitochondrion of mammalian cells, and are catalyzed by citrate synthase, aconitase, isocitrate dehydrogenase, α-ketoglutarate dehydrogenase, succinyl coenzyme A synthetase, succinate dehydrogenase, fumarase, and malate dehydrogenase.

Fermentation occurs when oxygen is limited. It converts pyruvate into lactic acid or ethanol. Fermentation is not as efficient as cellular respiration in converting nutrients into ATP. This process occurs in the cytoplasm.

Glycolysis does not only produce ATP, but also provides metabolic intermediates needed for cell growth and proliferation. In oncology, most cancer cells predominantly produce energy by a high rate of glycolysis followed by lactic acid fermentation in the cytosol—an observation called the Warburg effect. Tumor cells are highly proliferative and typically increase glycolytic rates by up to 200 times higher than those of their normal tissues of origin. This occurs even if oxygen is plentiful. In 1956, Otto Warburg postulated that elevation in glycolysis is the fundamental cause of cancer, a hypothesis currently known as the Warburg effect.

The Warburg effect describes the metabolic changes in a cell or tissue. Cells increase glycolysis with formation of lactate and decrease cellular respiration in mitochondria for the generation of ATP and recycling of NADH to NAD⁺. Accumulating evidence has shown that the Warburg effect is probably mediated by the master transcription factor hypoxia-inducible factor-1 (HIF-1α). In fact, several enzymes in glycolysis are upregulated by HIF-1α, such as aldolase, (Lu H, Forbes R A, Verma A. Hypoxia-inducible factor 1 activation by aerobic glycolysis implicates the Warburg effect in carcinogenesis. J Biol Chem. 2002 Jun. 28; 277(26):23111-5), triosephosphate isomerase (Gess B, Hofbauer K H, Deutzmann R, Kurtz A. Hypoxia up-regulates triosephosphate isomerase expression via a HIF-dependent pathway. Pflugers Arch. 2004 May; 448(2):175-80), and hexokinase (Riddle SR1, Ahmad A, Ahmad S, Deeb S S, Malkki M, Schneider B K, Allen C B, White C W. Hypoxia induces hexokinase II gene expression in human lung cell line A549. Am J Physiol Lung Cell Mol Physiol. 2000 February; 278(2):L407-16.). In addition to elevating glycolysis under hypoxia, HIF-1α also plays a regulatory role in inflammation. Expression of HIF-1α is regulated by proinflammatory cytokines, bacterial products, and microbial infection. At the same time, HIF-1αmediates production of IL-1β (Zhang W I, Petrovic J M, Callaghan D, Jones A, Cui H, Howlett C, Stanimirovic D. Evidence that hypoxia-inducible factor-1 (HIF-1) mediates transcriptional activation of interleukin-1beta (IL-1beta) in astrocyte cultures. J Neuroimmunol. 2006 May; 174(1-2):63-73). The interactions between HIF-1, glycolysis, and the immune response to microbes and their virulent factors still remains to be explored.

Assessing the severity of gingivitis and periodontitis is currently achieved with clinical measures such as gum redness, gum bleeding or pocket depth. While the measures are based on professionally developed scales, the actual values can vary due to examiner differences. There exists a need to quantify how severe gingivitis is and how effective treatments from oral hygiene products are in promoting gingivitis resolution. It is desirable to have objective readings from an instrument that is free of human errors. Transcriptomics, proteomics, and metabonomics measurements in saliva have been used to diagnose gingivitis, and to monitor progresses in treatment. But there is a disadvantage associated with saliva, in that the composition of saliva will be varied dependent upon the time of collection. As should be apparent, this field has a need for a more sensitive, accurate, and consistent test whenever an individual appear in a dentist office, or in a clinical setting, or at home.

### SUMMARY OF THE INVENTION

The foregoing summary is not intended to define every aspect of the invention, and additional aspects are described in other sections, such as the Detailed Description. In addition, the invention includes, as an additional aspect, all embodiments of the invention narrower in scope in any way than the variations defined by specific paragraphs set forth herein. For example, certain aspects of the invention that are described as a genus, and it should be understood that every member of a genus is, individually, an aspect of the invention. Also, aspects described as a genus or selecting a member of a genus should be understood to embrace combinations of two or more members of the genus. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

A method is provided for reducing a tetrazolium salt comprising providing an oral cavity sample; combining the oral cavity sample with a tetrazolium salt; wherein the oral cavity sample comprises an enzyme and at least one of a dehydrogenase, reductase or reducing reagent; and wherein the tetrazolium salt is reduced to produce a formazan dye.

A method is provided for reducing resazurin comprising providing an oral cavity sample; combining the oral cavity sample with resazurin; wherein the oral cavity sample comprises an enzyme and at least one of a dehydrogenase, reductase or reducing reagent; and wherein the resazurin is reduced to produce resorufin.

A method for determining the effectiveness of an oral care composition for maintaining oral health and/or showing the effects of an oral care composition upon gingival inflammation is provided that comprises acquiring an oral cavity sample before and after treatment with an oral care composition; combining the oral cavity sample with a tetrazolium salt; wherein the oral cavity sample comprises an enzyme and at least one of a dehydrogenase, reductase or reducing reagent; and wherein the tetrazolium salt is reduced to produce a formazan dye; or wherein resazurin is reduced to resorufin.

A method for detecting malate dehydrogenase and triosephosphate isomerase from oral biological samples is provided that comprises substrates, an electron coupling reagent, a cofactor and a tetrazolium salt.

### BRIEF DESCRIPTION OF THE DRAWINGS

FIG. 1. Graph showing Modified Gingival Index (MGI) presented by Adjusted Mean vs. Visit by Treatment.

FIG. 2A. Graph showing Gingival Bleeding Index (GBI) presented by Adjusted Mean vs. Visit by Treatment.

FIG. 2B. Graph showing the number of Bleeding Sites are presented by Adjusted Mean vs. Visit by Treatment.

FIG. 3A. Graph showing Spectrum of formazan dyes in the presence of diaphorase.

FIG. 3B. Graph showing Spectrum of formazan dyes in the presence of diaphorase.

FIG. 4A. Graph showing the effect of different concentrations of tetrazolium salts on formation of formazan dyes.

FIG. 4B. Graph showing the effect of different concentrations of tetrazolium salts on formation of formazan dyes.

FIG. 4C. Graph showing the effect of different concentrations of tetrazolium salts on formation of formazan dyes.

FIG. 4D. Graph showing the effect of different concentrations of tetrazolium salts on formation of formazan dyes.

FIG. 4E. Graph showing the effect of different concentrations of tetrazolium salts on formation of formazan dyes.

FIG. 4F. Graph showing the effect of different concentrations of tetrazolium salts on formation of formazan dyes.

FIG. 4G. Graph showing the effect of different concentrations of tetrazolium salts on formation of formazan dyes.

FIG. 5. Graph showing the effect of different concentrations of NAD+ on formation of formazan dyes.

FIG. 6. Graph showing the effect of different concentrations of malate on formation of formazan dyes.

FIG. 7. Graph showing the effect of different concentrations of malate dehydrogenase on formation of formazan dyes.

FIG. 8. Graph showing bleeding and inflammation results.

FIG. 9A Graph showing reduction activities (relative fluorescence unit) in oral lavage.

FIG. 9B Graph showing reduction activities (absorbance) in oral lavage.

### DETAILED DESCRIPTION OF THE INVENTION

The present invention includes methods of measuring the levels of a set of biomarkers in the gingiva. The set of biomarkers may include one or more metabolites, proteins, or messenger RNA (mRNA). Those metabolites and proteins have been shown to change in abundance at particular stages of treatment periods, or in in vitro models treated with different virulence factors, or human dental plaques. Accordingly, the set of metabolite biomarkers may be quantified to determine whether the gingiva has inflammation, whether the gingiva is under oxidative stresses or energy imbalance, and whether the gingiva has cellular damage or injuries.

The present invention demonstrates a role for metabolite and proteins biomarkers to serve as indicators of gingivitis at different stages, and indicators for gingival damage resulting from differing insults, such as oxidative stresses, high bacterial load, proinflammatory insults, energy imbalance or cellular injuries. The methods described herein demonstrate that either elevated or decreased levels of multiple metabolites and/or proteins can be used as a tool for accurately characterizing the quality of the gingiva, such as gingivitis.

Features of the compositions and methods are described below. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. It will be understood that any feature of the methods or compounds described herein can be deleted, combined with, or substituted for, in whole or part, any other feature described herein.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, liquids, gels, gel caps, tablets, and throat sprays; leave-on skin lotions and creams, shampoos, body washes, body rubs, such as Vicks Vaporub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

The term “dentifrice”, as used herein, includes tooth or subgingival—paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

As used herein, the term “oral cavity” means the part of the mouth including the teeth and gums and the cavity behind the teeth and gums that is bounded above by the hard and soft palates and below by the tongue and mucous membrane.

As used herein, the term “biomarker” means a substance that is objectively measured and evaluated as an indicator of normal biologic processes, pathogenic processes, treatment responses to chemical agents, or mechanical instruments. As used herein, biomarkers include, but are not limited to metabolites, proteins and messenger RNA (mRNA).

As used herein, the term “metabolite” means a substance that is objectively measured and evaluated as an indicator of normal biologic processes, pathogenic processes, treatment responses to chemical agents, or mechanical instruments; wherein said metabolites include, but are not limited to, a compound generated by lipid metabolism, protein metabolism, amino acid metabolism, carbohydrate metabolism, nuclear acid metabolism, or oxidative phosphorylation.

As used herein, the term “protein” means a substance that is objectively measured and evaluated as an indicator of normal biologic processes, pathogenic processes, treatment responses to chemical agents, or mechanical instruments; wherein the protein is a polymer consisting of more than three amino acids, including, but not limited to, enzymes, cytokines, chemokines, growth factors, cellular and extracellular proteins.

As used herein, the term “mRNA” means a substance that is a polymer of four ribonucleotides (adenine, uracil, guanine, cytosine), messenger RNA (mRNA) molecules convey genetic information from DNA to the ribosome, where they specify the amino acid sequence of the protein products of gene expression.

As used herein, the term “oral cavity sample” includes biological material isolated from one or more individuals; for example from gingivae, oral mucosa, mouth, supragingival space, or subgingival pockets, wherein gingival samples are isolated from gingivae, and buccal samples are isolated from oral mucosa; wherein oral lavage samples are collected from the mouth by rinsing the mouth with 3-6 ml of a selected solution, such as water; wherein gingival plaques are harvested from supragingival space and/or from subgingival pockets.

As used herein, the term “gum sensitivity” is a sensorial feeling, caused by activating transient receptor potential channel (TRP) V1 or TRPA1 on sensory neurons. Gum sensitivity is a common complaint due to inflammation, and can affect the area covering one or more teeth. Gum sensitivity is often noted when one eats or drinks something hot, cold, sweet, or sour; and can be experienced as a dull or sharp pain. The pain can begin suddenly and be felt deeply in the nerve endings of the tooth. Certain polyunsaturated fatty acids (PUFA), such as linoleic acid, arachidonic acid, hydroxyoctadecadienoic acid (HODE), and hydroxyeicosatetraenoic acid (HETE), are known to activate or sensitize TRPV1 and TRPA1. Certain oxidized lipids also activate TRPV1 and TRPA1 on sensory neurons, such as hydroxyoctadecadienoic acid (HODE) and hydroxyeicosatetraenoic acid (HETE), Prostaglandins, prostacyclins, and thromboxanes.

The term “low bleeder” refers to a panelist with three or less bleeding sites as assessed clinically from a dental probe pushed into the gingiva, generally referred to as bleeding on probing (BOP).

The term “high bleeder” refers to a panelist with twenty or more bleeding sites as determined clinically via BOP.

As used herein, the term “oxidative stress” is a threshold criteria based on panelists exhibiting an imbalance between the production of free radicals and the ability of the body to counteract or detoxify the reactive intermediates or to repair the resulting damage.

As used herein, the term “energy imbalance” or the term “mitochondrial dysfunction” means an imbalance of energy homeostasis. Mitochondria are found in every nucleated cell of the human body, and convert the energy of carbohydrate and fat into the ATP that powers most cellular functions. Both the citric acid cycle and β-oxidation of fatty acids are carried out in mitochondria. In gingivitis where gingivae are inflamed or damaged, AMP levels are high, meaning ATP production is impaired. Similarly, carnitine is a cofactor that helps carry fatty acid into mitochondria. Deoxycarnitine is an immediate precursor of carnitine.

As used herein, the term “glycolysis” means a series of biochemical reactions including, but not limited to, breakdown of glucose into pyruvate. It extends to include production of lactate and/or ethanol from pyruvate. Enzymes involved in the glycolysis process include hexokinase, phosphoglucose isomerase, phosphofructokinase, aldolase, triosephosphate isomerase, glyceraldehyde phosphate dehydrogenase, phosphoglycerate kinase, phosphoglycerate mutase, enolase, pyruvate dehydrogenase, lactate dehydrogenase and alcohol dehydrogenase.

As used herein, the term “cellular respiration” means a set of metabolic reactions to convert biochemical energy from nutrients into (ATP), carbon dioxide and water. This process includes three sub-pathways—pyruvate oxidation, the citric acid cycle and the electron transport chain. The citric acid cycle—also known as the tricarboxylic acid cycle (TCA cycle) and the Krebs cycle—is a series of enzyme-catalyzed catabolic reactions, breaking a six carbon molecule into a four carbon molecule and two molecules of carbon dioxides. The chemical reactions occur in the matrix of the mitochondrion of mammalian cells, and are catalyzed by citrate synthase, aconitase, isocitrate dehydrogenase, a-ketoglutarate dehydrogenase, succinyl coenzyme A synthetase, succinate dehydrogenase, fumarase, and malate dehydrogenase.

As used herein, the term “barrier function” means the defense function of epithelium against the environment, such as heat, dust, and microbes.

As used herein, the term “immunoassay” means any assay based on antibody-binding-to-specific targets, including, but not limiting to, ELISA (enzyme-linked immunosorbent assay) and immunoblotting. The targets can include, but are not limited to, proteins, peptides, fatty acids, carbohydrates, metabolites, and nucleic acids.

Certain embodiments of the present invention provide a method for collection of gingival brush samples. Gingival brush samples may be taken around a tooth or around the connecting areas between the gingiva and the tooth. In one or more embodiments, a collection device, such as an interdental gum brush or buccal brush may be used to collect gingival samples by swabbing back and forth multiple times with the brush-head oriented parallel to the gum line. A portion of the collection device that contacted the connecting areas between the gingiva and tooth may be detached and placed into a container; for example a brush head may be clipped off with a pair of sterile scissors and placed into a container, which may contain a buffer solution or an RNAlater solution.

As used herein, the term “oral lavage” means the fluid collected from the oral cavity. Oral lavage samples may be collected by rinsing the oval cavity with 4 ml of water for 30 seconds and then expectorating the contents of the mouth into a 15 ml centrifuge tube. Oral lavage contains both metabolites and proteins. Metabolites include, but are not limited to, malate, succinate, fumarate, lactate, and phosphoenolpyruvate, for example as shown in TABLE 24 herein. Those metabolites may be derived from glycolysis and citric acid cycle processes. Proteins in oral lavage samples may be composed of many enzymes, including lactate dehydrogenase, malate dehydrogenase, alcohol dehydrogenase and glyceraldehyde 3-phosphate dehydrogenase. They are involved in the glycolysis and citric acid cycle processes. Those enzymes can catalyze oxidation of the metabolites accompanied by reduction of NAD+ (oxidized nicotinamide adenine dinucleotide) into NADH (reduced nicotinamide adenine dinucleotide). In turn, NADH is oxidized into NAD+ accompanied by reduction of tetrazolium salts into formazan products. The latter display a variety of colors, such as yellow, purple and blue. Similarly, oxidization of NADH can also reduce resazurin (7-Hydroxy-3H-phenoxazin-3-one 10-oxide) into resorufin. Resazurin is a blue dye and weakly fluorescent. Upon reduction, resazurin is reduced to resorufin, which is pink and highly red fluorescent.

In certain embodiments of the present invention, a group of tetrazolium salts is used to detect the activities of enzymes that catalyze the biochemical reactions in glycolysis or cellular respiration. The tetrazolium salts are reduced by diaphorase to form formazan dyes in the presence of cofactors, examples of which include magnesium, rotenone, phosphate, and NADH (reduced nicotinamide adenine dinucleotide) or NADPH (reduced nicotinamide adenine dinucleotide phosphate). Enzymes in the oral lavage, gingival brush samples, and in supragingival and subgingival plaque samples can oxidize their relative substrates and also reduce NAD+ or NADP+ into NADH or NADPH. As a result, enzymes in the oral lavage samples, gingival brush samples, supragingival and subgingival samples can convert tetrazolium salts into formazan dyes in biochemical reactions containing malate, succinate, lactate, glycose, dihydroxyacetone phosphate, glyceraldehyde 3-phosphate, magnesium, rotenone, phosphate, NAD+, NADP and other related materials. The gingival brush samples from the unhealthy, gingivitis panelists contain more metabolic enzymes involved in the glycolysis and citric acid cycle processes and more metabolites derived from glycolysis and citric acid cycle processes than those of healthy panelists. Consequently, more enzymes in the gingivitis samples could elevate the conversion of NAD+ to NADH, and then increase reduction of tetrazolium salts and resazarin to formazan products and resorufin, respectively. As a result, more colored formazan and resorufin products are generated in gingivitis samples, forming the basis of diagnosis of gingivitis.

Tetrazolium salts are widely used for measuring the redox potential in biological samples, living cells and tissues. They are reduced to produce chromogenic formazan products by dehydrogenases, reductases and reducing agents. Formazan dyes display a broad spectrum of colors from dark blue, deep red, to orange, depending on the tetrazolium salt and the electron coupling reagents in the reaction. As used herein, the term “electron coupling reagent” means a material that mediates electron transfer between NADH or NADPH and various electron acceptors such as tetrazolium salts or resazurin. Electron coupling reagents include, but not limited to, 1-methoxy-5-methylphenazinium methyl sulfate (1-methoxyPMS), 5-methylphenazinium methyl sulfate (PMS), and diaphorase. Major tetrazolium salts include MTT (3-(4,5-dimethyl-2-thiazolyl)-2,5-diphenyl-2H-tetrazolium bromide), INT (2-(4-Iodophenyl)-3-(4-nitrophenyl)-5-phenyl-2H-tetrazolium chloride), TTC (2,3,5-Triphenyl-2H-tetrazolium chloride), MTS (3-(4,5-dimethylthiazol-2-yl)-5-(3-carboxymethoxyphenyl)-2-(4-sulfophenyl)-2H-tetrazolium), XTT (2,3-bis-(2-methoxy-4-nitro-5-sulfophenyl)-2H-tetrazolium-5-carboxanilide), and NBT (2,2′-bis(4-Nitrophenyl)-5,5′-diphenyl-3,3′-(3,3′-dimethoxy-4,4′-diphenylene) ditetrazolium chloride 3,3′-(3,3′-Dimethoxy-4,4′-biphenylene)bis[2-(4-nitrophenyl)-5-phenyl-2H-tetrazolium chloride]), MTS, 3-(4,5-dimethylthiazol-2-yl)-5-(3-carboxymethoxyphenyl)-2-(4-sulfophenyl)-2H-tetrazolium, inner salt.

In certain embodiments of the present invention a list of proteins has been identified which are either higher or lower in concentrations in the oral lavage of a high bleeder group than that of a low bleeder group. Similarly, a group of proteins has been discovered which are either increased or decreased after panelists with gingivitis were treated with a regimen. Proteins and enzymes which can be used in the methods of this invention include those listed in TABLE 1 and TABLE 23. Oral lavage may comprise microbial products, microbial toxins, live and dead microbes, mucosal fluid, gingival crevicular fluid, epithelial cells and their secreted products, infiltrated blood cells and their products, and secretions from salivary glands. Thus, there are a number of highly complex interactions amongst these various components that compose oral lavage. Undoubtedly, oral lavage can all be impacted differentially on the overall oral health status of the epithelium lining the oral cavity.

### EXAMPLES

All EXAMPLES were run at room temperature (RT), standard pressure and atmosphere, unless otherwise noted. The water used in the EXAMPLES was deionized water, unless otherwise noted.

### Example 1

### A Method to Collect Oral Lavage to Assess Changes in Gingivitis-Related Molecular Markers

Assessing the degree of gingivitis in a person is generally done by a qualified examiner using clinical measures, such as gum redness, gum bleeding or pocket depth. While the measures are based on professionally developed scales, the actual values can vary due to differences between examiners. To reduce or remove these variances it is desirable to have objective readings from instruments that are free of differences between human examiners. The sample collection described below is quantifiable objective measurement of the degree of gingivitis.

A clinical study was conducted to evaluate sample collection methods and measurement procedures. It was a controlled, examiner-blind study. Forty panelists satisfying the inclusion/exclusion criteria were enrolled. Twenty (20) panelists were qualified as healthy—with up to 3 bleeding sites and with all pockets less than or equal to 2 mm deep and twenty (20) panelists were qualified as unhealthy—greater than 20 bleeding sites with at least 3 pockets greater than or equal to 3 mm but not deeper than 4 mm with bleeding, and at least 3 pockets less than or equal to 2 mm deep with no bleeding for sampling. All panelists had up to 6 sites identified as “sampling sites”. Sampling sites had supragingival and subgingival plaque collected at Baseline, Week 2 and Week 4, as described below. Supragingival and subgingival plaque samples were taken from a gingival sulcus of the pre-identified sites.

Supragingival Plague Sample: Plaque samples were collected using a sterile curette at each site. Samples were taken at the tooth/gum interface (supragingival gumline and interproximal, buccal surfaces only) using care to avoid contact with the oral soft tissues. Plaques were transferred to pre-labeled tubes. Supragingival samples were stored at −80° C. freezer until analysis.

Subgingival Sample: Subgingival plaque samples were taken from a gingival sulcus from the pre-identified bleeding and nonbleeding sites. Prior to sample collection, the site had supragingival plaque removed with a curette. The site was dried and subgingival plaque samples were collected with another dental curette. Samples from each site were placed in a pre-labeled 2.0 ml sterile tube containing PBS buffer with glass beads. Samples were stored at −80° C. until analysis.

Metabonomics: The samples were thawed at room temperature and dispersed in a TissueLyser II (Qiagen, Valencia, Calif., USA) at 30 shakes per second for 3 min Protein concentrations of the dispersed subgingival samples were measured using a Pierce microBCA Protein kit (ThermoFisher Scientific, Grand Island, N.Y., USA) following the manufacturer's instruction.

Oral lavage samples were collected at wake up (one per panelist) by rinsing with 4 ml of water for 30 seconds and then expectorating the contents of the mouth into a centrifuge tube. These samples were frozen at home until they were brought into a test site in a cold pack. Each panelist provided up to 15 samples throughout the study. Oral lavage samples at a test site were frozen at −70° C.

All panelists were given investigational products: Crest® Pro-Health Clinical Gum Protection Toothpaste (0.454% stannous fluoride) and Oral-B® Indicator Soft Manual Toothbrush. Panelists continued their regular oral hygiene routine, and did not use any new products starting from the baseline to the end of four week treatment study. During the four week treatment period, panelists brushed their teeth twice daily, morning and evening, in their customary manner using the assigned dentifrice and soft manual toothbrush.

### Example 2

### Changes of Modified Gingival Index (MGI) and Gingival Bleeding Index (GBI) after Four Week Application of Pro-Health Clinical Gum Protection Toothpaste

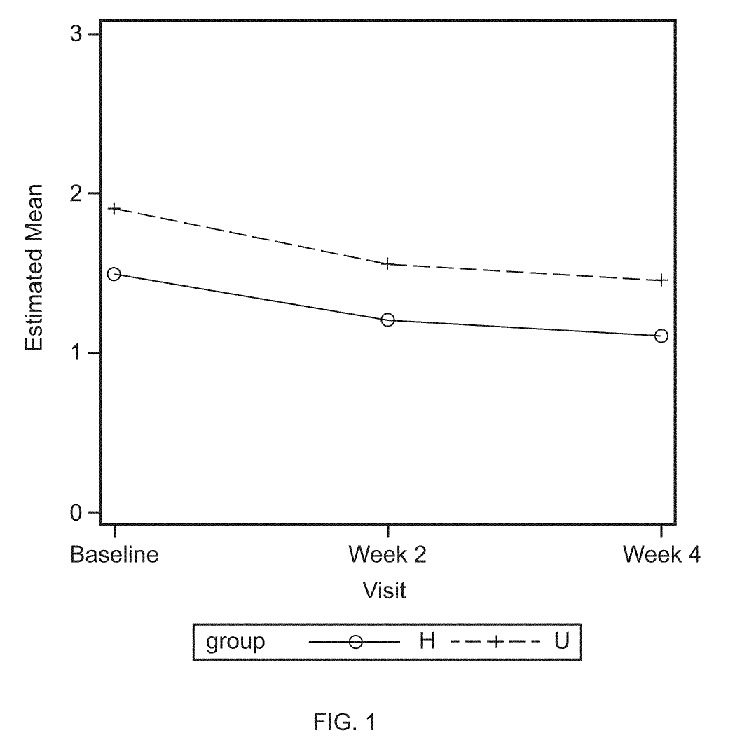

The clinical study was carried out with two groups of panelists as described in Example 1: low bleeders (healthy, non-gingivitis) and high bleeders (chronic gingivitis, unhealthy). All panelists used investigative products for four weeks, as described in Example 1. Modified gingival index (MGI) and gingival bleeding index (GBI) were determined prior to application of the investigative products (baseline), and at week 2 and week 4 of application of the investigative products. MGI was higher in the unhealthy (high bleeder) panelists than the healthy panelists (low bleeders), represented by U and H, respectively, in FIG. 1. MGI was reduced, as compared to baseline, during week 2 and 4 of application of the investigative products for both healthy and unhealthy panelists.

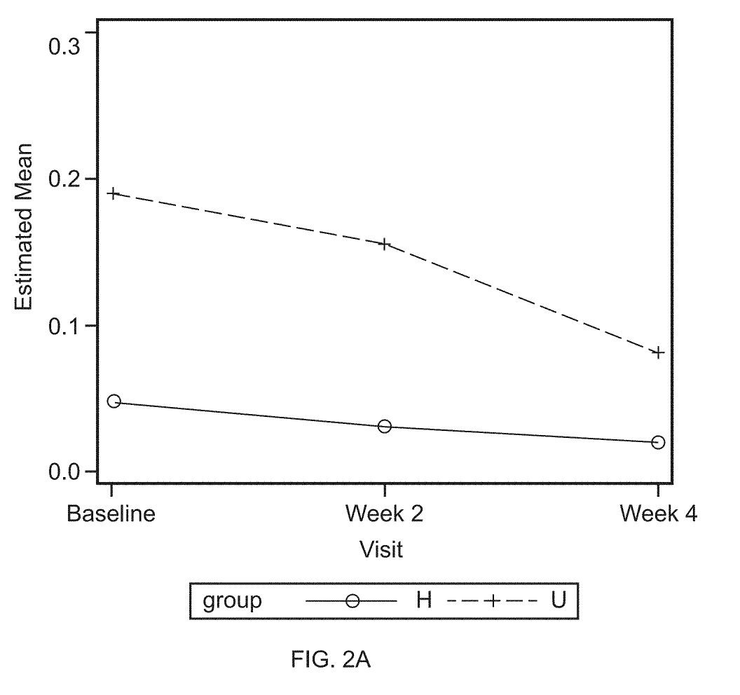

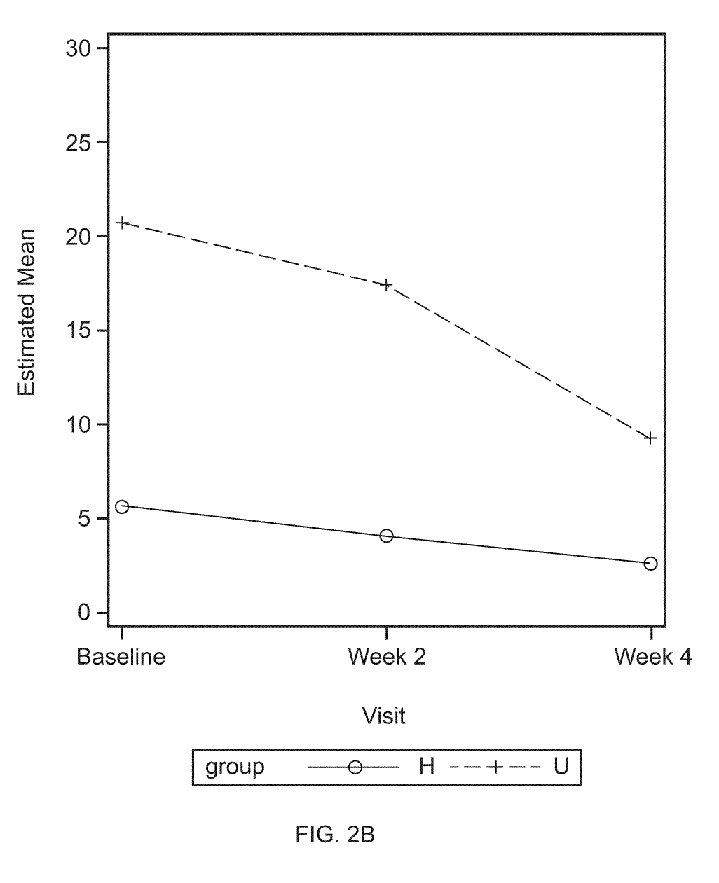

Similarly, gingival bleeding index (GBI) was higher in the unhealthy (high bleeder) panelists than the healthy panelists (low bleeders), represented by U and H, respectively, in FIG. 2A and 2B. GBI and the number of bleeding sites were reduced during week 2 and 4 of application of the investigative products for both healthy and unhealthy panelists.

### Example 3

### Proteins in Oral Lavage

Oral lavage samples were collected, as described as in Example 1, before treatment (baseline) and at the end of a four week application of investigative products. The oral lavage samples were divided into four groups: Low bleeder baseline, Low bleeder week 4, High bleeder baseline, and High bleeder week 4. Each group consists of 20 samples. Ten samples from each of the three sets of samples, including Low bleeder baseline, High bleeder baseline, and High bleeder week 4, were sent to SomaLogic, Inc. (Boulder, Colo.) for protein measurement.

Oral lavage contains proteins secreted from gingival epithelium, oral mucosa, infiltrated neutrophils, lymphocytes, and monocytes of blood. In addition, it also includes microbial proteins.

As shown in TABLE 1, enzymes involved in glycolysis, such as Glucose-6-phosphate isomerase, Fructose-bisphosphate aldolase A, triosephosphate isomerase, and Glyceraldehyde-3-phosphate dehydrogenase, Phosphoglycerate kinase 1, Phosphoglycerate mutase 1, were far more abundant in the oral lavage of high bleeders at baseline than of the low bleeders.

The biochemical profiles of oral lavage from 20 panelists with gingivitis (unhealthy, high bleeders) and 20 non-gingivitis (low bleeders) panelists were analyzed, prior to and following a 4 week toothpaste treatment. As can be seen in TABLE 1, many proteins were significantly (p≤0.05) different in concentrations between high and low bleeder panelists at baseline. Similarly, many proteins were found to be different in concentrations in the gingival brush samples between baseline and three weeks of treatment (TABLE 23). Some enzymes were found to be changed in concentrations in both oral lavage and gingival brush samples, such as triosephosphate isomerase, and malate dehydrogenase.

TABLE 1
Abundance of proteins in human oral lavage.

| | | | | Fold Change | | p-value | |
|---|---|---|---|---|---|---|---|
| | | | | High | High | High | High |
| | | | | bleeder | bleeder | bleeder | bleeder |
| | Means | | | W4 | BL | W4 | BL |
| | (Original Scale) | | | vs | vs | vs | vs |
| | Low | High | High | High | Low | High | Low |
| | bleeder | bleeder | bleeder | bleeder | bleeder | bleeder | bleeder |
| Proteins | BL | BL | W4 | BL | BL | BL | BL |
| 14-3-3 protein theta | 838 | 5344 | 2603 | 0.57 | 5.73 | 0.08 | 0.00 |
| 26S proteasome non-ATPase | 80 | 368 | 252 | 0.74 | 3.81 | 0.04 | 0.00 |
| regulatory subunit 7 | | | | | | | |
| 3-hydroxyanthranilate 3,4- | 1631 | 13609 | 7138 | 0.56 | 8.00 | 0.06 | 0.00 |
| dioxygenase | | | | | | | |
| 40S ribosomal protein S7 | 67 | 198 | 141 | 0.73 | 2.50 | 0.01 | 0.01 |
| 40S ribosomal protein SA | 185 | 665 | 418 | 0.71 | 3.24 | 0.05 | 0.00 |
| 60 kDa heat shock protein, | 236 | 421 | 319 | 0.79 | 1.86 | 0.03 | 0.02 |
| mitochondrial | | | | | | | |
| 72 kDa type IV collagenase | 1249 | 4284 | 2573 | 0.61 | 3.20 | 0.03 | 0.00 |
| Adenylosuccinate lyase | 241 | 1990 | 1160 | 0.63 | 6.74 | 0.07 | 0.00 |
| ADP-ribosyl cyclase/cyclic | 14521 | 35309 | 21260 | 0.59 | 2.53 | 0.01 | 0.01 |
| ADP-ribose hydrolase 2 | | | | | | | |
| Agouti-related protein | 33 | 53 | 44 | 0.85 | 1.55 | 0.05 | 0.00 |
| Alanine aminotransferase 1 | 3829 | 14877 | 10506 | 0.71 | 4.42 | 0.02 | 0.00 |
| Alcohol dehydrogenase | 4061 | 34583 | 17004 | 0.37 | 22.75 | 0.20 | 0.00 |
| [NADP(+)] | | | | | | | |
| Alpha-(1,3)-fucosyltransferase 5 | 1516 | 8483 | 5258 | 0.82 | 5.83 | 0.57 | 0.01 |
| Alpha-1-antitrypsin | 1399 | 3598 | 1784 | 0.62 | 2.26 | 0.05 | 0.05 |
| Alpha-2-HS-glycoprotein | 3367 | 23244 | 12828 | 0.66 | 6.01 | 0.11 | 0.00 |
| Alpha-enolase | 110398 | 217066 | 170325 | 0.76 | 2.28 | 0.04 | 0.01 |
| Amphiregulin | 72 | 168 | 108 | 0.71 | 2.24 | 0.03 | 0.01 |
| Amyloid beta A4 protein | 51294 | 128850 | 81350 | 0.68 | 2.42 | 0.03 | 0.01 |
| Angiotensinogen | 13227 | 39831 | 25334 | 0.95 | 6.12 | 0.88 | 0.03 |
| Annexin A6 | 2676 | 7520 | 4233 | 0.62 | 3.10 | 0.01 | 0.01 |
| Antithrombin-III | 1424 | 7388 | 2485 | 0.95 | 5.80 | 0.89 | 0.03 |
| Arylsulfatase A | 1559 | 5748 | 3387 | 0.58 | 4.58 | 0.03 | 0.00 |
| Aspartate aminotransferase, | 2562 | 8249 | 4985 | 0.62 | 3.15 | 0.01 | 0.02 |
| cytoplasmic | | | | | | | |
| ATP synthase subunit beta, | 162 | 526 | 265 | 0.58 | 2.70 | 0.00 | 0.01 |
| mitochondrial | | | | | | | |
| ATP synthase subunit O, | 331 | 469 | 325 | 0.70 | 1.47 | 0.02 | 0.10 |
| mitochondrial | | | | | | | |
| ATP-dependent RNA helicase | 55 | 220 | 116 | 0.63 | 3.17 | 0.02 | 0.00 |
| DDX19B | | | | | | | |
| Bactericidal permeability- | 23709 | 134635 | 77909 | 0.41 | 5.35 | 0.02 | 0.00 |
| increasing protein | | | | | | | |
| B-cell lymphoma 6 protein | 5292 | 17925 | 6602 | 0.39 | 2.22 | 0.00 | 0.10 |
| Bone morphogenetic protein 7 | 32 | 66 | 50 | 0.80 | 1.90 | 0.05 | 0.00 |
| Brevican core protein | 364 | 3617 | 1786 | 0.51 | 9.85 | 0.04 | 0.00 |
| C3a anaphylatoxin des | 3702 | 32149 | 17327 | 0.64 | 8.20 | 0.15 | 0.00 |
| Arginine | | | | | | | |
| Cadherin-5 | 252 | 1720 | 807 | 0.53 | 5.83 | 0.02 | 0.00 |
| Calcineurin | 311 | 2092 | 1313 | 0.71 | 5.64 | 0.29 | 0.00 |
| Calcineurin subunit B type 1 | 2009 | 12624 | 4434 | 0.53 | 4.97 | 0.05 | 0.00 |
| Calpain I | 25144 | 112397 | 50592 | 0.51 | 5.97 | 0.02 | 0.00 |
| cAMP-dependent protein | 272 | 3400 | 998 | 0.63 | 7.15 | 0.24 | 0.01 |
| kinase catalytic subunit alpha | | | | | | | |
| Carbohydrate sulfotransferase | 71 | 713 | 348 | 0.60 | 8.61 | 0.19 | 0.00 |
| 15 | | | | | | | |
| Carbonic anhydrase 6 | 161029 | 198844 | 226319 | 1.15 | 1.25 | 0.01 | 0.03 |
| Caspase-10 | 2648 | 11989 | 6828 | 0.64 | 4.05 | 0.04 | 0.00 |
| Caspase-2 | 64 | 110 | 88 | 0.82 | 1.68 | 0.02 | 0.00 |
| Caspase-3 | 555 | 2391 | 1368 | 0.59 | 5.96 | 0.06 | 0.00 |
| Cathepsin B | 16350 | 16857 | 8354 | 0.53 | 1.01 | 0.00 | 0.98 |
| Cathepsin F | 1136 | 8192 | 3442 | 0.59 | 4.94 | 0.04 | 0.01 |
| Cathepsin S | 2396 | 18016 | 9075 | 0.65 | 7.16 | 0.13 | 0.00 |
| Cation-independent mannose- | 10151 | 35875 | 22743 | 0.68 | 3.20 | 0.05 | 0.00 |
| 6-phosphate receptor | | | | | | | |
| CD109 antigen | 233 | 512 | 302 | 0.61 | 2.24 | 0.01 | 0.01 |
| CD166 antigen | 2616 | 7301 | 4033 | 0.61 | 2.50 | 0.02 | 0.01 |
| CD209 antigen | 76 | 262 | 176 | 0.70 | 3.22 | 0.05 | 0.00 |
| CD83 antigen | 52 | 130 | 78 | 0.66 | 2.28 | 0.03 | 0.01 |
| Chitotriosidase-1 | 14176 | 58999 | 41470 | 0.70 | 7.00 | 0.09 | 0.01 |
| Chloride intracellular channel | 108 | 787 | 349 | 0.61 | 5.88 | 0.22 | 0.00 |
| protein 1 | | | | | | | |
| Choline/ethanolamine kinase | 243 | 525 | 400 | 0.78 | 2.11 | 0.01 | 0.00 |
| Chorionic | 1638 | 10909 | 6769 | 0.70 | 5.58 | 0.13 | 0.00 |
| somatomammotropin hormone | | | | | | | |
| Clusterin | 340 | 1890 | 1193 | 0.65 | 4.12 | 0.03 | 0.01 |
| Coactosin-like protein | 512 | 2271 | 1213 | 0.60 | 3.84 | 0.01 | 0.00 |
| Cofilin-1 | 254 | 1143 | 622 | 0.62 | 3.94 | 0.01 | 0.00 |
| Collagen alpha-1(XXIII) chain | 106 | 289 | 174 | 0.64 | 2.61 | 0.01 | 0.01 |
| Complement C1q | 3534 | 31801 | 19342 | 0.89 | 8.65 | 0.75 | 0.00 |
| subcomponent | | | | | | | |
| Complement C1r | 918 | 7672 | 2780 | 0.52 | 8.87 | 0.05 | 0.00 |
| subcomponent | | | | | | | |
| Complement C2 | 455 | 4609 | 1511 | 0.64 | 6.34 | 0.25 | 0.01 |
| Complement C4 | 9814 | 53505 | 34190 | 0.73 | 5.62 | 0.21 | 0.00 |
| Complement C5 | 1080 | 7106 | 3434 | 0.77 | 7.04 | 0.47 | 0.01 |
| Complement component C9 | 7900 | 76942 | 31536 | 0.73 | 18.85 | 0.43 | 0.00 |
| Complement decay- | 85178 | 136366 | 106648 | 0.77 | 1.59 | 0.03 | 0.01 |
| accelerating factor | | | | | | | |
| Connective tissue-activating | 311 | 2483 | 678 | 0.47 | 4.08 | 0.03 | 0.04 |
| peptide III | | | | | | | |
| Contactin-1 | 1919 | 9982 | 5517 | 0.60 | 4.20 | 0.04 | 0.00 |
| Contactin-5 | 281 | 937 | 612 | 0.63 | 3.15 | 0.03 | 0.00 |
| C-reactive protein | 285 | 2929 | 1665 | 0.72 | 12.47 | 0.29 | 0.00 |
| Creatine kinase M- | 53 | 105 | 74 | 0.75 | 1.97 | 0.04 | 0.01 |
| type:Creatine kinase B-type | | | | | | | |
| heterodimer | | | | | | | |
| Cryptic protein | 478 | 1592 | 654 | 0.46 | 3.42 | 0.00 | 0.00 |
| C-type mannose receptor 2 | 716 | 2270 | 1308 | 0.63 | 2.98 | 0.03 | 0.00 |
| C-X-C motif chemokine 6 | 26 | 132 | 45 | 0.63 | 2.45 | 0.05 | 0.03 |
| Cyclin-dependent kinase | 89 | 246 | 167 | 0.69 | 2.58 | 0.02 | 0.00 |
| inhibitor 1B | | | | | | | |
| Cystatin-M | 6730 | 17308 | 8283 | 0.50 | 2.72 | 0.00 | 0.01 |
| Cystatin-SA | 215101 | 216303 | 225660 | 1.04 | 1.01 | 0.00 | 0.86 |
| Cysteine and glycine-rich | 69 | 270 | 167 | 0.71 | 3.18 | 0.05 | 0.00 |
| protein 3 | | | | | | | |
| Cytoskeleton-associated | 1303 | 5329 | 3399 | 0.68 | 3.90 | 0.03 | 0.00 |
| protein 2 | | | | | | | |
| D-dimer | 2569 | 19261 | 8828 | 0.66 | 5.99 | 0.21 | 0.01 |
| Desmocollin-3 | 201 | 752 | 317 | 0.53 | 2.67 | 0.01 | 0.03 |
| Desmoglein-1 | 3634 | 12244 | 4796 | 0.44 | 3.45 | 0.00 | 0.00 |
| Diablo homolog, mitochondrial | 366 | 1487 | 827 | 0.60 | 3.96 | 0.01 | 0.00 |
| Disintegrin and | 113 | 768 | 530 | 0.74 | 6.05 | 0.31 | 0.00 |
| metalloproteinase domain- | | | | | | | |
| containing protein 9 | | | | | | | |
| DNA topoisomerase 1 | 79 | 399 | 193 | 0.59 | 4.09 | 0.04 | 0.00 |
| Drebrin-like protein | 809 | 2088 | 1305 | 0.67 | 2.37 | 0.01 | 0.00 |
| Dual specificity mitogen- | 34 | 96 | 69 | 0.74 | 2.47 | 0.01 | 0.00 |
| activated protein kinase kinase 1 | | | | | | | |
| Dual specificity mitogen- | 100 | 244 | 158 | 0.70 | 2.18 | 0.03 | 0.00 |
| activated protein kinase kinase 4 | | | | | | | |
| E3 ubiquitin-protein ligase | 40 | 78 | 57 | 0.77 | 1.95 | 0.03 | 0.01 |
| Mdm2 | | | | | | | |
| EGF-containing fibulin-like | 3473 | 29110 | 11693 | 0.50 | 6.80 | 0.05 | 0.00 |
| extracellular matrix protein 1 | | | | | | | |
| Endoglin | 31 | 55 | 38 | 0.72 | 1.65 | 0.04 | 0.01 |
| Endoplasmic reticulum | 1909 | 9255 | 5865 | 1.04 | 13.26 | 0.92 | 0.01 |
| aminopeptidase 1 | | | | | | | |
| Endothelial cell-selective | 1083 | 2472 | 1391 | 0.56 | 2.25 | 0.01 | 0.00 |
| adhesion molecule | | | | | | | |
| Endothelial monocyte- | 468 | 2955 | 1541 | 0.60 | 4.67 | 0.02 | 0.00 |
| activating polypeptide 2 | | | | | | | |
| Ephrin type-A receptor 1 | 375 | 605 | 371 | 0.59 | 2.13 | 0.02 | 0.04 |
| Ephrin type-A receptor 2 | 16730 | 47551 | 26305 | 0.59 | 2.80 | 0.02 | 0.00 |
| Ephrin type-B receptor 2 | 986 | 3011 | 1883 | 0.52 | 2.36 | 0.01 | 0.04 |
| Ephrin type-B receptor 6 | 392 | 1464 | 755 | 0.54 | 3.50 | 0.03 | 0.00 |
| Ephrin-A4 | 350 | 1384 | 729 | 0.55 | 3.60 | 0.04 | 0.00 |
| Ephrin-B1 | 1760 | 5419 | 3253 | 0.64 | 2.92 | 0.03 | 0.00 |
| Ephrin-B2 | 1236 | 2152 | 1295 | 0.59 | 1.91 | 0.01 | 0.01 |
| Epidermal growth factor | 4786 | 40558 | 18414 | 0.55 | 18.15 | 0.15 | 0.00 |
| Epidermal growth factor | 4814 | 12139 | 7779 | 0.62 | 2.72 | 0.02 | 0.01 |
| receptor | | | | | | | |
| Epiregulin | 365 | 923 | 545 | 0.68 | 2.08 | 0.02 | 0.03 |
| Fatty acid-binding protein, | 3121 | 6011 | 3062 | 0.53 | 2.38 | 0.04 | 0.04 |
| heart | | | | | | | |
| Fibroblast growth factor 10 | 32 | 52 | 41 | 0.82 | 1.56 | 0.01 | 0.00 |
| Fibronectin | 19501 | 81632 | 58452 | 0.92 | 6.50 | 0.80 | 0.01 |
| Ficolin-1 | 329 | 5106 | 1286 | 0.57 | 6.22 | 0.13 | 0.01 |
| Formimidoyltransferase- | 89 | 202 | 110 | 0.59 | 2.01 | 0.02 | 0.01 |
| cyclodeaminase | | | | | | | |
| Fructose-bisphosphate aldolase A | 33774 | 255824 | 179719 | 0.68 | 18.59 | 0.08 | 0.00 |
| Galectin-10 | 75 | 349 | 146 | 0.49 | 3.52 | 0.00 | 0.00 |
| Galectin-7 | 219 | 697 | 435 | 0.68 | 2.83 | 0.01 | 0.00 |
| Gamma-enolase | 200 | 420 | 244 | 0.62 | 2.36 | 0.05 | 0.02 |
| Glucose-6-phosphate | 1781 | 22949 | 20969 | 1.57 | 10.27 | 0.51 | 0.07 |
| isomerase | | | | | | | |
| Glutamate carboxypeptidase 2 | 38 | 72 | 137 | 1.89 | 1.83 | 0.01 | 0.01 |
| Glutathione S-transferase P | 39413 | 49555 | 36219 | 0.69 | 1.64 | 0.02 | 0.10 |
| Glyceraldehyde-3-phosphate | 12144 | 106769 | 85859 | 0.44 | 14.20 | 0.10 | 0.00 |
| dehydrogenase | | | | | | | |
| Granulocyte colony- | 121 | 504 | 268 | 0.61 | 3.52 | 0.03 | 0.00 |
| stimulating factor | | | | | | | |
| Granulocyte colony- | 166 | 327 | 219 | 0.70 | 1.90 | 0.03 | 0.00 |
| stimulating factor receptor | | | | | | | |
| Granulocyte-macrophage | 1747 | 3974 | 2161 | 0.61 | 2.41 | 0.04 | 0.09 |
| colony-stimulating factor | | | | | | | |
| Growth arrest-specific protein 1 | 433 | 2909 | 1272 | 0.49 | 5.48 | 0.02 | 0.00 |
| Growth/differentiation factor 5 | 260 | 511 | 423 | 0.80 | 1.89 | 0.04 | 0.00 |
| Growth-regulated alpha protein | 270 | 3896 | 649 | 0.48 | 5.15 | 0.05 | 0.02 |
| GTP-binding nuclear protein | 254 | 5086 | 2502 | 0.55 | 22.19 | 0.23 | 0.00 |
| Ran | | | | | | | |
| Haptoglobin | 68133 | 128447 | 99492 | 0.71 | 2.07 | 0.01 | 0.07 |
| Heat shock 70 kDa protein 1A | 16620 | 87728 | 63420 | 0.67 | 6.33 | 0.12 | 0.00 |
| Heat shock protein beta-1 | 84 | 216 | 124 | 0.61 | 2.28 | 0.01 | 0.00 |
| Heat shock protein HSP 90- | 1417 | 31849 | 8692 | 0.47 | 18.08 | 0.21 | 0.00 |
| alpha/beta | | | | | | | |
| Heat shock protein HSP 90- | 13991 | 114383 | 53401 | 0.61 | 11.49 | 0.27 | 0.00 |
| beta | | | | | | | |
| HemK methyltransferase | 689 | 2638 | 1424 | 0.62 | 3.51 | 0.02 | 0.00 |
| family member 2 | | | | | | | |
| Hemopexin | 662 | 2677 | 1383 | 0.64 | 6.00 | 0.14 | 0.01 |
| Hepatocyte growth factor-like | 131 | 2399 | 580 | 0.61 | 9.38 | 0.19 | 0.00 |
| protein | | | | | | | |
| HERV-H LTR-associating | 226 | 1034 | 623 | 0.64 | 4.01 | 0.03 | 0.00 |
| protein 2 | | | | | | | |
| High affinity nerve growth | 428 | 919 | 590 | 0.67 | 2.06 | 0.04 | 0.00 |
| factor receptor | | | | | | | |
| Histone H1.2 | 14 | 29 | 18 | 0.68 | 1.93 | 0.01 | 0.04 |
| Histone-lysine N- | 79 | 224 | 154 | 0.72 | 2.52 | 0.02 | 0.00 |
| methyltransferase EHMT2 | | | | | | | |
| ICOS ligand | 13720 | 80395 | 51085 | 0.73 | 5.66 | 0.23 | 0.00 |
| Iduronate 2-sulfatase | 738 | 1631 | 1106 | 0.70 | 2.21 | 0.01 | 0.00 |
| Immunoglobulin M | 31428 | 97097 | 62055 | 0.61 | 3.51 | 0.02 | 0.01 |
| Importin subunit alpha-1 | 52 | 182 | 84 | 0.53 | 3.19 | 0.00 | 0.00 |
| Inhibitor of growth protein 1 | 369 | 1046 | 592 | 0.58 | 2.67 | 0.02 | 0.00 |
| Inorganic pyrophosphatase | 1342 | 4082 | 1645 | 0.49 | 3.73 | 0.01 | 0.02 |
| Insulin-like growth factor I | 1140 | 6949 | 2357 | 0.45 | 4.98 | 0.02 | 0.00 |
| Insulin-like growth factor- | 13120 | 36296 | 21609 | 0.65 | 2.49 | 0.05 | 0.01 |
| binding protein 5 | | | | | | | |
| Insulin-like growth factor- | 761 | 2178 | 1237 | 0.63 | 2.37 | 0.05 | 0.03 |
| binding protein 6 | | | | | | | |
| Insulin-like growth factor- | 578 | 2441 | 760 | 0.39 | 3.09 | 0.01 | 0.01 |
| binding protein 7 | | | | | | | |
| Integrin alpha-I: beta-1 | 948 | 11211 | 6351 | 0.58 | 6.43 | 0.07 | 0.01 |
| complex | | | | | | | |
| Intercellular adhesion molecule 2 | 507 | 4810 | 1913 | 0.55 | 6.58 | 0.06 | 0.00 |
| Interferon gamma | 87 | 658 | 263 | 0.53 | 5.99 | 0.04 | 0.00 |
| Interferon regulatory factor 1 | 165 | 2361 | 954 | 0.64 | 7.66 | 0.10 | 0.00 |
| Interleukin-1 alpha | 1194 | 1989 | 1071 | 0.51 | 1.86 | 0.00 | 0.06 |
| Interleukin-1 beta | 152 | 397 | 228 | 0.60 | 2.54 | 0.01 | 0.00 |
| Interleukin-1 Receptor | 1501 | 5433 | 2418 | 0.52 | 3.72 | 0.02 | 0.00 |
| accessory protein | | | | | | | |
| Interleukin-1 receptor | 142737 | 169211 | 155231 | 0.91 | 1.20 | 0.04 | 0.01 |
| antagonist protein | | | | | | | |
| Interleukin-1 receptor-like 2 | 506 | 1304 | 898 | 0.67 | 2.67 | 0.04 | 0.00 |
| Interleukin-24 | 65 | 111 | 87 | 0.81 | 1.70 | 0.03 | 0.00 |
| Interleukin-27 | 46 | 130 | 82 | 0.69 | 2.65 | 0.02 | 0.00 |
| Interleukin-3 receptor subunit | 89 | 190 | 120 | 0.66 | 2.03 | 0.01 | 0.00 |
| alpha | | | | | | | |
| Interleukin-36 beta | 3545 | 8814 | 5962 | 0.68 | 2.65 | 0.04 | 0.00 |
| Interleukin-6 receptor subunit | 21961 | 63627 | 40305 | 0.62 | 2.76 | 0.03 | 0.00 |
| beta | | | | | | | |
| Kallikrein-12 | 523 | 22605 | 9720 | 0.76 | 6.84 | 0.38 | 0.04 |
| Kallikrein-13 | 16522 | 53398 | 32056 | 0.65 | 3.40 | 0.04 | 0.00 |
| Kallikrein-6 | 280 | 1014 | 468 | 0.55 | 3.37 | 0.01 | 0.00 |
| Kallikrein-8 | 15860 | 33249 | 19220 | 0.67 | 1.84 | 0.01 | 0.10 |
| Kelch-like ECH-associated | 82 | 312 | 187 | 0.63 | 3.50 | 0.01 | 0.00 |
| protein 1 | | | | | | | |
| Kininogen-1 | 13797 | 95635 | 35597 | 0.52 | 8.91 | 0.08 | 0.01 |
| Kunitz-type protease inhibitor 1 | 2020 | 4290 | 2333 | 0.55 | 2.09 | 0.00 | 0.04 |
| Kunitz-type protease inhibitor 2 | 304 | 414 | 303 | 0.76 | 1.32 | 0.00 | 0.22 |
| Latent-transforming growth | 770 | 3512 | 1923 | 0.57 | 4.42 | 0.05 | 0.00 |
| factor beta-binding protein 4 | | | | | | | |
| Layilin | 342 | 635 | 374 | 0.61 | 1.89 | 0.00 | 0.01 |
| Legumain | 23410 | 65072 | 41251 | 0.66 | 2.73 | 0.01 | 0.00 |
| Leptin | 179 | 952 | 526 | 0.61 | 4.44 | 0.02 | 0.00 |
| Leukemia inhibitory factor | 228 | 1001 | 562 | 0.58 | 3.76 | 0.02 | 0.00 |
| receptor | | | | | | | |
| Lipopolysaccharide-binding | 305 | 6694 | 1807 | 0.37 | 13.95 | 0.02 | 0.00 |
| protein | | | | | | | |
| Lithostathine-1-alpha | 5643 | 7884 | 4725 | 0.58 | 1.81 | 0.03 | 0.17 |
| L-lactate dehydrogenase B | 20548 | 269414 | 220227 | 0.88 | 20.02 | 0.58 | 0.00 |
| chain | | | | | | | |
| Low-density lipoprotein | 197 | 477 | 292 | 0.62 | 2.52 | 0.02 | 0.00 |
| receptor-related protein 1, | | | | | | | |
| soluble | | | | | | | |
| Low-density lipoprotein | 3273 | 31383 | 24064 | 0.94 | 7.51 | 0.81 | 0.00 |
| receptor-related protein 1B | | | | | | | |
| Lumican | 5119 | 35877 | 11354 | 0.42 | 6.31 | 0.01 | 0.01 |
| Ly6/PLAUR domain- | 138269 | 166337 | 140608 | 0.83 | 1.23 | 0.01 | 0.03 |
| containing protein 3 | | | | | | | |
| Macrophage colony- | 2422 | 6274 | 4163 | 0.66 | 2.61 | 0.03 | 0.00 |
| stimulating factor 1 | | | | | | | |
| Macrophage mannose receptor 1 | 223 | 852 | 434 | 0.58 | 3.34 | 0.05 | 0.01 |
| Macrophage metalloelastase | 2096 | 16571 | 5282 | 0.48 | 6.43 | 0.05 | 0.00 |
| Malate dehydrogenase, | 12672 | 256310 | 185328 | 0.41 | 208.97 | 0.29 | 0.00 |
| cytoplasmic | | | | | | | |
| Matrilin-2 | 2992 | 17458 | 8756 | 0.63 | 7.28 | 0.12 | 0.00 |
| Matrilysin | 3498 | 25019 | 19110 | 1.05 | 6.34 | 0.89 | 0.00 |
| Mitogen-activated protein | 129 | 1159 | 457 | 0.65 | 6.92 | 0.33 | 0.01 |
| kinase 1 | | | | | | | |
| Mitogen-activated protein | 55 | 239 | 108 | 0.58 | 3.23 | 0.02 | 0.00 |
| kinase 11 | | | | | | | |
| Mitogen-activated protein | 596 | 5862 | 3068 | 0.60 | 15.65 | 0.38 | 0.00 |
| kinase 14 | | | | | | | |
| Mitogen-activated protein | 305 | 2149 | 953 | 0.62 | 6.58 | 0.30 | 0.01 |
| kinase 3 | | | | | | | |
| Mitogen-activated protein | 1367 | 2969 | 1342 | 0.57 | 1.74 | 0.00 | 0.05 |
| kinase 9 | | | | | | | |
| Muellerian-inhibiting factor | 671 | 1634 | 1097 | 0.72 | 2.57 | 0.03 | 0.01 |
| Myc proto-oncogene protein | 42 | 71 | 54 | 0.77 | 1.67 | 0.04 | 0.01 |
| N-acetyl-D-glucosamine | 283 | 2839 | 1570 | 0.48 | 12.44 | 0.13 | 0.00 |
| kinase | | | | | | | |
| N-acylethanolamine- | 121 | 1737 | 606 | 0.52 | 6.62 | 0.04 | 0.00 |
| hydrolyzing acid amidase | | | | | | | |
| NAD-dependent protein | 3074 | 7030 | 4631 | 0.67 | 2.21 | 0.00 | 0.00 |
| deacetylase sirtuin-2 | | | | | | | |
| NADPH--cytochrome P450 | 221 | 1621 | 1355 | 0.54 | 10.71 | 0.18 | 0.00 |
| reductase | | | | | | | |
| Natural cytotoxicity triggering | 45 | 77 | 55 | 0.76 | 1.66 | 0.01 | 0.01 |
| receptor 2 | | | | | | | |
| Netrin-1 | 71 | 321 | 167 | 0.64 | 3.82 | 0.04 | 0.00 |
| Neuregulin-1 | 107 | 251 | 142 | 0.60 | 2.07 | 0.02 | 0.01 |
| Neurexophilin-1 | 135 | 377 | 224 | 0.66 | 2.43 | 0.01 | 0.00 |
| Neurogenic locus notch | 2264 | 12232 | 5959 | 0.54 | 4.76 | 0.02 | 0.00 |
| homolog protein 3 | | | | | | | |
| Neutrophil collagenase | 9187 | 21211 | 4713 | 0.48 | 2.96 | 0.03 | 0.24 |
| Neutrophil gelatinase- | 209403 | 290631 | 182049 | 0.55 | 1.71 | 0.01 | 0.13 |
| associated lipocalin | | | | | | | |
| Neutrophil-activating peptide 2 | 259 | 2147 | 569 | 0.45 | 4.62 | 0.03 | 0.04 |
| Nidogen-1 | 157 | 526 | 329 | 0.65 | 3.12 | 0.04 | 0.00 |
| NSFL1 cofactor p47 | 570 | 1799 | 924 | 0.57 | 3.16 | 0.03 | 0.00 |
| Nucleoside diphosphate kinase A | 6942 | 14841 | 8700 | 0.69 | 2.30 | 0.02 | 0.02 |
| Nucleoside diphosphate kinase B | 342 | 1205 | 913 | 0.76 | 6.98 | 0.17 | 0.00 |
| Osteocalcin | 986 | 3206 | 1808 | 0.63 | 3.38 | 0.03 | 0.01 |
| Osteomodulin | 301 | 2109 | 736 | 0.51 | 4.01 | 0.02 | 0.03 |
| Oxidized low-density | 10913 | 68060 | 43398 | 0.79 | 7.00 | 0.46 | 0.00 |
| lipoprotein receptor 1 | | | | | | | |
| Parathyroid hormone | 32 | 97 | 58 | 0.67 | 2.54 | 0.02 | 0.00 |
| Parathyroid hormone-related | 45 | 102 | 74 | 0.76 | 2.19 | 0.02 | 0.00 |
| protein | | | | | | | |
| Peptidyl-prolyl cis-trans | 41428 | 237334 | 210485 | 0.86 | 14.17 | 0.71 | 0.00 |
| isomerase A | | | | | | | |
| Peptidyl-prolyl cis-trans | 534 | 9535 | 4115 | 0.58 | 17.73 | 0.21 | 0.00 |
| isomerase F, mitochondrial | | | | | | | |
| Peroxiredoxin-1 | 30663 | 62705 | 33782 | 0.53 | 2.55 | 0.03 | 0.02 |
| Peroxiredoxin-6 | 9404 | 22914 | 15898 | 0.64 | 2.62 | 0.02 | 0.03 |
| Phosphoglycerate mutase 1 | 7725 | 52081 | 19944 | 0.17 | 25.56 | 0.08 | 0.00 |
| Plasma kallikrein | 4040 | 46557 | 15193 | 0.73 | 9.09 | 0.46 | 0.01 |
| Plasma protease C1 inhibitor | 1321 | 13980 | 8339 | 0.79 | 5.77 | 0.46 | 0.02 |
| Plasminogen activator inhibitor 1 | 39 | 100 | 67 | 0.72 | 2.40 | 0.05 | 0.00 |
| Platelet factor 4 | 131 | 405 | 200 | 0.61 | 2.33 | 0.04 | 0.01 |
| Platelet receptor Gi24 | 2622 | 7220 | 4528 | 0.62 | 2.74 | 0.01 | 0.00 |
| Platelet-activating factor | 1218 | 6029 | 3019 | 0.55 | 4.50 | 0.02 | 0.00 |
| acetylhydrolase IB subunit beta | | | | | | | |
| Pleiotrophin | 9417 | 20915 | 10949 | 0.58 | 2.15 | 0.01 | 0.03 |
| Plexin-B2 | 32750 | 72614 | 47158 | 0.61 | 2.28 | 0.01 | 0.00 |
| PolyUbiquitin K63-linked | 10517 | 27725 | 14663 | 0.54 | 3.06 | 0.01 | 0.00 |
| Prefoldin subunit 5 | 349 | 1191 | 775 | 0.65 | 3.12 | 0.02 | 0.00 |
| Properdin | 7086 | 52072 | 29032 | 0.81 | 5.80 | 0.43 | 0.01 |
| Proteasome activator complex | 84 | 180 | 96 | 0.64 | 1.85 | 0.03 | 0.02 |
| subunit 3 | | | | | | | |
| Proteasome subunit alpha type-1 | 2767 | 18422 | 8281 | 0.41 | 8.33 | 0.00 | 0.00 |
| Proteasome subunit alpha type-2 | 1058 | 3457 | 1924 | 0.53 | 4.39 | 0.00 | 0.01 |
| Proteasome subunit alpha type-6 | 139 | 392 | 224 | 0.65 | 2.77 | 0.02 | 0.01 |
| Protein deglycase DJ-1 | 788 | 1197 | 455 | 0.36 | 2.67 | 0.00 | 0.03 |
| Protein E7_HPV18 | 200 | 493 | 249 | 0.66 | 2.26 | 0.04 | 0.03 |
| Protein FAM107B | 115 | 190 | 157 | 0.84 | 1.65 | 0.02 | 0.00 |
| Protein S100-A12 | 42367 | 90312 | 37426 | 0.38 | 2.33 | 0.00 | 0.02 |
| Protein S100-A7 | 2514 | 12300 | 5172 | 0.25 | 1.89 | 0.00 | 0.27 |
| Protein S100-A9 | 25886 | 39861 | 15320 | 0.30 | 2.27 | 0.00 | 0.13 |
| Prothrombin | 4336 | 27146 | 10978 | 0.56 | 6.53 | 0.08 | 0.00 |
| Proto-oncogene tyrosine- | 224 | 1690 | 697 | 0.51 | 6.85 | 0.09 | 0.00 |
| protein kinase Src | | | | | | | |
| Pyridoxal kinase | 281 | 2457 | 1778 | 0.54 | 12.00 | 0.38 | 0.01 |
| Rab GDP dissociation inhibitor | 49425 | 173349 | 149926 | 0.90 | 6.49 | 0.61 | 0.00 |
| beta | | | | | | | |
| RAC-alpha/beta/gamma | 166 | 602 | 268 | 0.54 | 3.77 | 0.03 | 0.00 |
| serine/threonine-protein kinase | | | | | | | |
| Ras-related C3 botulinum toxin | 3799 | 37672 | 20914 | 0.91 | 9.95 | 0.81 | 0.00 |
| substrate 1 | | | | | | | |
| Repulsive guidance molecule A | 2620 | 10483 | 5917 | 0.62 | 3.69 | 0.04 | 0.00 |
| RGM domain family member B | 1389 | 9860 | 4375 | 0.52 | 6.17 | 0.03 | 0.00 |
| Ribosomal protein S6 kinase | 12 | 27 | 18 | 0.73 | 2.12 | 0.03 | 0.00 |
| alpha-5 | | | | | | | |
| Ribosome maturation protein | 230 | 595 | 263 | 0.60 | 2.55 | 0.04 | 0.02 |
| SBDS | | | | | | | |
| RNA-binding protein 39 | 276 | 2206 | 1128 | 0.48 | 6.91 | 0.06 | 0.00 |
| Secreted and transmembrane | 400 | 1633 | 905 | 0.64 | 3.57 | 0.03 | 0.00 |
| protein 1 | | | | | | | |
| Secreted frizzled-related | 1212 | 4666 | 2482 | 0.59 | 3.68 | 0.02 | 0.00 |
| protein 1 | | | | | | | |
| Semaphorin-6A | 396 | 3637 | 2503 | 0.68 | 11.58 | 0.18 | 0.00 |
| Serine protease 27 | 7376 | 10968 | 6186 | 0.55 | 1.83 | 0.00 | 0.03 |
| Serine/threonine-protein kinase | 138 | 350 | 231 | 0.73 | 2.31 | 0.04 | 0.01 |
| 16 | | | | | | | |
| Serine/threonine-protein kinase | 115 | 348 | 231 | 0.69 | 2.84 | 0.03 | 0.00 |
| PAK 7 | | | | | | | |
| Serum amyloid P-component | 17509 | 83820 | 38594 | 0.54 | 6.00 | 0.01 | 0.00 |
| S-formylglutathione hydrolase | 122 | 455 | 283 | 0.62 | 4.02 | 0.03 | 0.00 |
| Sialic acid-binding Ig-like | 1489 | 1602 | 996 | 0.70 | 1.43 | 0.02 | 0.37 |
| lectin 14 | | | | | | | |
| Signal transducer and activator | 554 | 5042 | 2798 | 0.60 | 9.28 | 0.18 | 0.00 |
| of transcription 3 | | | | | | | |
| Signal transducer and activator | 438 | 2367 | 2358 | 0.79 | 7.44 | 0.43 | 0.00 |
| of transcription 6 | | | | | | | |
| SLAM family member 7 | 561 | 996 | 712 | 0.76 | 1.66 | 0.04 | 0.01 |
| Small nuclear | 143 | 755 | 413 | 0.62 | 4.40 | 0.02 | 0.00 |
| ribonucleoprotein F | | | | | | | |
| Small ubiquitin-related | 15775 | 42271 | 24878 | 0.63 | 3.03 | 0.04 | 0.00 |
| modifier 3 | | | | | | | |
| Somatostatin-28 | 48 | 171 | 96 | 0.65 | 3.00 | 0.02 | 0.00 |
| SPARC-related modular | 8240 | 25985 | 17574 | 0.66 | 2.94 | 0.04 | 0.00 |
| calcium-binding protein 1 | | | | | | | |
| S-phase kinase-associated | 1572 | 3493 | 1881 | 0.57 | 1.97 | 0.01 | 0.01 |
| protein 1 | | | | | | | |
| Stabilin-2 | 140 | 291 | 205 | 0.75 | 2.19 | 0.05 | 0.01 |
| Stanniocalcin-1 | 896 | 11768 | 3794 | 0.45 | 9.04 | 0.04 | 0.00 |
| Stress-induced-phosphoprotein 1 | 4297 | 16420 | 9806 | 0.53 | 5.59 | 0.03 | 0.00 |
| Stromelysin-2 | 217 | 2793 | 1746 | 0.45 | 8.04 | 0.05 | 0.00 |
| SUMO-conjugating enzyme | 14235 | 69920 | 44705 | 0.68 | 6.93 | 0.09 | 0.00 |
| UBC9 | | | | | | | |
| Superoxide dismutase [Cu—Zn] | 1625 | 1468 | 489 | 0.29 | 1.49 | 0.00 | 0.40 |
| Superoxide dismutase [Mn], | 14640 | 25375 | 18580 | 0.72 | 1.86 | 0.01 | 0.00 |
| mitochondrial | | | | | | | |
| Tenascin | 1484 | 11450 | 4204 | 0.59 | 6.06 | 0.09 | 0.00 |
| Testican-1 | 3587 | 23537 | 13734 | 0.61 | 6.23 | 0.02 | 0.00 |
| Thioredoxin domain- | 7236 | 33968 | 18352 | 0.57 | 6.16 | 0.04 | 0.00 |
| containing protein 12 | | | | | | | |
| Thrombospondin-4 | 235 | 1702 | 608 | 0.52 | 5.93 | 0.05 | 0.00 |
| Tissue Factor | 106 | 174 | 133 | 0.78 | 1.64 | 0.01 | 0.01 |
| T-lymphocyte surface antigen | 481 | 1717 | 877 | 0.55 | 3.27 | 0.02 | 0.01 |
| Ly-9 | | | | | | | |
| Transcription factor IIIB 90 kDa | 118 | 362 | 255 | 0.74 | 2.92 | 0.03 | 0.00 |
| subunit | | | | | | | |
| Transforming growth factor- | 209 | 770 | 446 | 0.62 | 3.15 | 0.02 | 0.00 |
| beta-induced protein ig-h3 | | | | | | | |
| Transgelin-2 | 43038 | 117595 | 74493 | 0.62 | 3.36 | 0.03 | 0.00 |
| Triosephosphate isomerase | 13987 | 199036 | 137411 | 0.63 | 15.87 | 0.11 | 0.00 |
| Tropomyosin alpha-4 chain | 3524 | 21810 | 7848 | 0.38 | 6.73 | 0.01 | 0.00 |
| Troponin I, cardiac muscle | 58 | 304 | 158 | 0.63 | 3.57 | 0.01 | 0.00 |
| Trypsin-1 | 423 | 1882 | 1012 | 0.61 | 3.94 | 0.02 | 0.03 |
| Trypsin-2 | 125 | 335 | 221 | 0.66 | 2.30 | 0.03 | 0.01 |
| Tumor necrosis factor receptor | 198 | 437 | 255 | 0.58 | 2.01 | 0.00 | 0.00 |
| superfamily member 14 | | | | | | | |
| Tumor necrosis factor receptor | 63 | 208 | 119 | 0.65 | 2.82 | 0.02 | 0.00 |
| superfamily member 18 | | | | | | | |
| Tumor necrosis factor receptor | 5149 | 15394 | 8811 | 0.65 | 2.68 | 0.05 | 0.01 |
| superfamily member 21 | | | | | | | |
| Tumor necrosis factor receptor | 586 | 1977 | 1099 | 0.60 | 3.04 | 0.04 | 0.00 |
| superfamily member 6 | | | | | | | |
| Tyrosine-protein kinase CSK | 522 | 4148 | 1086 | 0.39 | 14.80 | 0.13 | 0.00 |
| Tyrosine-protein kinase Lyn | 107 | 727 | 457 | 0.55 | 10.17 | 0.15 | 0.00 |
| Tyrosine-protein kinase Lyn, | 567 | 4095 | 2475 | 0.53 | 15.37 | 0.19 | 0.00 |
| isoform B | | | | | | | |
| Tyrosine-protein kinase | 50 | 95 | 67 | 0.74 | 1.82 | 0.03 | 0.00 |
| receptor TYRO3 | | | | | | | |
| Tyrosine-protein phosphatase | 670 | 5104 | 3229 | 0.66 | 12.44 | 0.04 | 0.00 |
| non-receptor type substrate 1 | | | | | | | |
| Ubiquitin carboxyl-terminal | 2687 | 8790 | 4911 | 0.62 | 3.47 | 0.02 | 0.00 |
| hydrolase isozyme L1 | | | | | | | |
| Ubiquitin-conjugating enzyme | 355 | 1599 | 890 | 0.62 | 4.04 | 0.02 | 0.00 |
| E2 G2 | | | | | | | |
| Ubiquitin-fold modifier- | 2760 | 7435 | 3140 | 0.48 | 2.23 | 0.01 | 0.14 |
| conjugating enzyme 1 | | | | | | | |
| Vascular endothelial growth | 1882 | 5151 | 3283 | 0.66 | 2.62 | 0.03 | 0.00 |
| factor A, isoform 121 | | | | | | | |
| Vascular endothelial growth | 61 | 296 | 144 | 0.54 | 4.24 | 0.02 | 0.00 |
| factor D | | | | | | | |
| Vesicular integral-membrane | 107 | 689 | 356 | 0.62 | 4.42 | 0.03 | 0.00 |
| protein VIP36 | | | | | | | |
| Vitamin K-dependent protein S | 3768 | 26044 | 11517 | 0.49 | 5.90 | 0.04 | 0.00 |
| Vitronectin | 648 | 1831 | 1169 | 0.70 | 2.55 | 0.01 | 0.00 |
| von Willebrand factor | 1535 | 10477 | 4655 | 0.84 | 6.12 | 0.63 | 0.02 |
| WNT1-inducible-signaling | 63 | 194 | 123 | 0.70 | 2.78 | 0.03 | 0.00 |
| pathway protein 3 | | | | | | | |
| X-linked interleukin-1 receptor | 112 | 347 | 224 | 0.70 | 2.77 | 0.04 | 0.00 |
| accessory protein-like 2 | | | | | | | |

### Example 4

### Malate Dehydrogenase, Triosephosphate Isomerase and Catalase Activities in the Oral Lavage

Oral lavage samples were collected, as described in Example 1, before treatment (baseline) and at the end of four week application of investigative products. The oral lavage samples were divided into four groups: Low bleeder baseline, Low bleeder week 4, High bleeder baseline, and High bleeder week 4. Each group consisted of 20 samples. All oral lavage samples were analyzed for malate dehydrogenase activities using malate dehydrogenase activity assay kit following manufacturer's instructions (Abcam, Cambridge, Mass.). All reagents were provided in the assay kit, including malate dehydrogenase assay buffer, enzyme mix, developer and substrate. A reaction buffer was prepared by adding 62 μl of malate dehydrogenase assay buffer, 2 μl of enzyme mix, 10 μl of developer, and 2 μl substrate to a well in a 96-well plate. Ten μl of oral lavage samples were finally added to the well. The reaction plate was set at room temperature for an hour, and absorbance was measured at 450 nM in a spectrometry plate reader (Spectra Max M3, Molecular Devices, Sunnyvale, Calif.).

As shown in TABLE 2, the activity of malate dehydrogenase in the oral lavage was higher at baseline in the high bleeder group than the low bleeder group. Treatment with investigative products (Crest® Pro-Health Clinical Gum Protection Toothpaste with 0.454% stannous fluoride and Oral-B® Indicator Soft Manual Tooth blush) reduced the activity at baseline in the high bleeder group.

TABLE 2
Malate dehydrogenase activity: absorbance was measured
at 450 nM at 60 min after substrates were added.

| Group | |
|---|---|
| High bleeder | Low bleeder |
| OD at 60 Min | OD at 60 Min |

| Time Point | Mean | Std Err | Mean | Std Err |
|---|---|---|---|---|
| Baseline | 0.40 | 0.04 | 0.27 | 0.02 |
| Week 4 | 0.30 | 0.02 | 0.27 | 0.02 |

All oral lavage samples were also analyzed for triosephosphate isomerase (TPI) activities using triosephosphate isomerase assay kit following manufacturer's instructions (BioVision, Inc. Milpitas, Calif.). All reagents were provided in the assay kit, including TPI assay buffer, enzyme mix, developer and substrate. A reaction buffer was prepared by adding 84 μl TPI assay buffer, 2 μl enzyme mix, 2 μl developer, and 2 μl substrate to a well in a 96-well plate. Ten μl of oral lavage samples were finally added to the well. The reaction plate was set at room temperature for an hour, and absorbance was measured at 450 nM in a spectrometry plate reader (Spectra Max M3, Molecular Devices, Sunnyvale, Calif.).

As shown in TABLE 3, the activity of triosephosphate isomerase in the oral lavage was higher at baseline in the high bleeder group than the low bleeder group. Treatment with investigative products (Crest® Pro-Health Clinical Gum Protection Toothpaste with 0.454% stannous fluoride and Oral-B® Indicator Soft Manual Tooth blush) reduced the activity at baseline in the high bleeder group.

TABLE 3
Triose phosphate isomerase activity: absorbance was measured
at 450 nM at 60 min after substrates were added.

| | |
|---|---|
| High bleeder | Low bleeder |
| OD at 10 min | OD at 10 min |

| Time Point | Mean | Std Err | Mean | Std Err |
|---|---|---|---|---|
| Baseline | 0.25 | 0.03 | 0.15 | 0.01 |
| Week 4 | 0.19 | 0.02 | 0.14 | 0.01 |

All oral lavage samples were analyzed for catalase activities using catalase activity assay kit following manufacturer's instructions (BioVision, Inc. Milpitas, Calif.). Briefly, all reagents were provided in the assay kit, including catalase assay buffer, OxiRed probe, horseradish peroxidase, hydrogen peroxide, and stop solution. Ten μl of oral lavage samples were first added to the wells in a 96-well plate. Then 12 μl of 1 mM hydrogen peroxide was added. The plate was set at 25° C. for 30 min. Next 10 μl stop solution was added to stop the reaction. To develop the color, a developer mix was added. The developer mix contained 2 μl OxiRed probe, 2 μl horseradish peroxidase, and 64 μl assay buffer. The reaction was carried out at 25° C. for 10 min, and products formed in the reaction were measured at 570 nM in a plate reader (Spectra Max M3, Molecular Devices, Sunnyvale, Calif.). Catalase activities were calculated as nmol/min/mL of hydrogen peroxide in the test samples following manufacturer's instruction.

As shown in TABLE 4, the activities of catalases in the oral lavage were higher at baseline in the high bleeder group than the low bleeder group. Treatment with investigative products (Crest® Pro-Health Clinical Gum Protection Toothpaste with 0.454% stannous fluoride and Oral-B® Indicator Soft Manual Tooth blush) reduced the activity at baseline in the high bleeder group.

TABLE 4
Catalase activity: absorbance was measured at 570 nM. Catalase
activity was calculated at nmol/min/mL of hydrogen peroxide.

| | |
|---|---|
| High bleeder | Low bleeder |
| Catalase Activity | Catalase Activity |
| nmol/min/mL | nmol/min/mL |

| Time Point | Mean | Std Err | Mean | Std Err |
|---|---|---|---|---|
| Baseline | 39.52 | 7.41 | 29.06 | 5.25 |
| Week 4 | 29.51 | 6.38 | 26.88 | 5.23 |

### Example 5

### Characterization of Tetrazolium Salts in Color Formation

A group of water-soluble tetrazolium salts (WSTs), including WST-1, 3, 4, 5, 8, 9, 10 and 11, were developed by introducing positive or negative charges and hydroxy groups to the phenyl ring of the tetrazolium salt. Those WSTs are easily reduced with NADH or other reducing agents to give orange or purple formazan dyes. Recently, a new water soluble tetrazolium was synthesized, and it is called EZMTT (Zhang W, Zhu M, Wang F, Cao D, Ruan J J, Su W, Ruan B H. Mono-sulfonated tetrazolium salt based NAD(P)H detection reagents suitable for dehydrogenase and real-time cell viability assays. Anal Biochem. 2016 Sep. 15; 509:33-40. doi: 10.1016/j.ab.2016.06.026. Epub 2016 Jul. 4). This new tetrazolium salt gives rise to orange color when reduced to form formazan dyes.

MTT assay is commonly used to determine cell viability, cell proliferation, and drug toxicity. MTT can enter into mitochondria and be reduced directly without any help from electron coupling agents. It can also be reduced by cytoplasmic dehydrogenases and reductases. When reduced in a cell, MTT forms an insoluble dark blue precipitate.

INT can also be used to measure cell viability in the presence of an electron coupling agent. It is usually used to determine activities of various dehydrogenases and reductases, which convert NAD to NADH, or NADP to NADPH. INT is reduced to form a cherry red formazan product. TTC is used to determine metabolic activities in cells and tissue. It's often employed to differentiate between metabolically active and inactive tissues. The white compound is enzymatically reduced to red formazan salts (1,3,5-triphenylformazan) in living tissues by dehydrogenases and reductases. However, it remains as white TTC in necrotic tissues which are deficient in active dehydrogenases and reductases. This color difference renders the TTC dye popular in heart research for identification of infarcted tissue caused by acute myocardial ischemia.

NBT (nitro-blue tetrazolium chloride) is widely employed in immunologic assays for detection of alkaline phosphatase. The combination of NBT and BCIP (5-bromo-4-chloro-3′-indolyphosphate p-toluidine salt) yields an intense, insoluble black-purple precipitate when reacted with alkaline phosphatase, a popular enzyme conjugate for antibody probes. Here, NBT serves as the chromogenic substrate and BCIP is the substrate for alkaline phosphate.

MTS assay was used to quantify cell numbers, based on the conversion of a tetrazolium salt into a colored, aqueous soluble formazan product by mitochondrial activity of viable cells. The amount of formazan produced by dehydrogenases and reductases is directly proportional to the number of metabolically active cells in culture. The MTS assay reagents were composed of solutions of MTS and an electron coupling reagent (PMS, phenazine methosulfate), which is required as a redox intermediary.

Another electron coupling reagent 1-methoxy phenazinium methylsulfate (PMS) is widely used as an electron carrier for NAD(P)H-tetrazolium reactions. It is easily dissolved in water and alcohol. Its redox potential is +63 mV. 1-methoxy PMS solution can be stored at room temperature for over 3 months without protection from light. Therefore, it is a useful regent for NAD(P)H-tetrazolium-based assay systems. Diaphorase, another electron coupling reagent, is often used to catalyze the transfer of electrons from NAD(P)H to tetrazolium salts.

To optimize assay conditions for detecting redox potentials of oral lavage samples, various tetrazolium salts were characterized in the presence of either diaphorase or 1-methoxy PMS. The assay system contained 0-100 units of diaphorase, 0-100 units of malate dehydrogenase, 1-300 mM malate, 0-80 mM NAD+, 0-40 mM NADH, 1-20 mM MgCl2, 0.1-20 mM Tetrazolium salts and 0-4 mM 1-methoxy-5-methylphenazinium methyl sulfate (1-methoxy PMS) in potassium phosphate 100 mM, pH 7.5. Diaphorase from Clostridium kluyveri, L-malate dehydrogenase (pig heart), NADH, MTT, INT, 1-methoxy PMS, XTT, NBT, TTC and NAD were purchased from Sigma-Aldrich (St. Louis, Mo.) as shown in TABLE 5, Potassium Phosphate Stock Solution (500 mM, pH 7.0) and Potassium Phosphate Stock Solution (500 mM, pH 8.0) were purchase from Cayman Chemical Company (Ann Arbor, Mich.). WST-1, 4, 5, 8, and 9 were purchased from Dojindo Molecular Technologies, Inc. (Rockville, Md.). The assay was run at room temperature for up to 24 hours in a kinetic mode. The absorbance reading was taken in every 30 or 60 min in a spectrometry plate reader (Spectra Max M3, Molecular Devices, Sunnyvale, Calif.).

TABLE 5

| COMPOUND/CHEMICAL | FUNCTION | VENDOR | CAT # |
|---|---|---|---|
| Diaphorase (Clostridium kluyveri) | Electron | Cayman | 14671 - 1 kU |
| | Carrier | | |
| DIAPHORASE FROM CLOSTRIDIUM KLUYVERI | Electron | Sigma | D5540- |
| | Carrier | | 500UN |
| Iodonitrotetrazolium chloride: 2-(4-Iodophenyl)-3-(4- | Dye | Sigma | 10406-5 G |
| nitrophenyl)-5-phenyl-2H-tetrazolium chloride, p- | | | |
| Iodonitrotetrazolium Violet, INT | | | |
| Iodonitrotetrazolium (chloride): 2-(4-iodophenyl)-3-(4- | Dye | Cayman | 16073 - 5 g |
| nitrophenyl)-5-phenyl-2H-tetrazolium, monochloride | | | |
| Magnesium Chloride (anhydrous) | Buffer | Sigma | M2670-500 G |
| Mallic Acid Sodium Salt | Buffer | Sigma | M1125-100 G |
| 1-Methoxy PMS: 1-Methoxy-5-methylphenazinium methyl | Electron | Sigma | M8640- |
| sulfate | Carrier | | 100 MG |
| L-malate dehydrogenase, (PIG HEART) | Control | Sigma | 10127248001 |
| | | | 5 MG |
| L-malate dehydrogenase, (PIG HEART), 25 MG | Control | Sigma | 10127914001 |
| Lipoamide dehydrogenase | Electron | Calzyme | 153A0025 |
| | Carrier | | |
| MTS: 3-(4,5-dimethylthiazol-2-yl)-5-(3- | Dye | Bio Vision | 2808-250 |
| carboxymethoxyphenyl)-2-(4-sulfophenyl)-2H-tetrazolium, | | | |
| inner salt | | | |
| MTT: 3-(4,5-Dimethyl-2-thiazolyl)-2,5-diphenyl-2H- | Dye | Dojindo | M009 |
| tetrazolium bromide | | | |
| NADH, APPROX. 100% (nicotinamide adenine dinucleotide | Control | Sigma | 10107735001 |
| (NAD) + hydrogen (H)) | | | |
| NAD, APPROX. 100%, GRADE I, LYO.5 G (Nicotinamide | Control | Sigma | 10127973001 |
| adenine dinucleotide) | | | |
| Nitro Blue Tetrazolium: 3,3′-[3,3′-Dimethoxy-(1,1′- | Dye | Sigma | N5514- |
| biphenyl)-4,4′-diyl]-bis[2-(4-nitrophenyl)-5-phenyl-2H- | | | 25TAB |
| tetrazolium chloride | | | |
| Nitro-TB: 3,3′-[3,3′-Dimethoxy-(1,1′-biphenyl)-4,4′-diyl]- | Dye | Dojindo | N011 |
| bis[2-(4-nitrophenyl)-5-phenyl-2H-tetrazolium chloride | | | |
| OXALOACETIC ACID 1PC X 5 GM | — | Sigma | 5000-5 GM |
| Phenazine Methosulfate | Electron | Sigma | P9625-10 G |
| | Carrier | | |
| Phenazine Ethosulfate | Electron | Sigma | P4544-1 G |
| | Carrier | | |
| Potassium Phosphate Stock Solution (500 mM, pH 7.0) | Buffer | Cayman | 600208 - 500 |
| | | | mL |
| Potassium Phosphate Stock Solution (500 mM, pH 8.0) | Buffer | Cayman | 600209 - 500 |
| | | | mL |
| Tetrazolium Violet: 2,5-Diphenyl-3-(α-naphthyl)tetrazolium | Dye | Sigma | T0138-1 G |
| chloride, 2,5-Diphenyl-3-(1-naphthyl)tetrazolium chloride, | | | |
| TV | | | |
| Triosephosphate Isomerase: D-Glyceraldehyde-3-phosphate | — | Sigma | T2507-10 MG |
| ketol-isomerase, TPI | | | |
| TTC: 2,3,5-Triphenyl-tetrazolium chloride solution | Dye | Sigma | 17779-10 ML- |
| | | | F |
| WST-1: 2-(4-Iodophenyl)-3-(4-nitrophenyl)-5-(2,4- | Dye | Bio Vision | 2198-30 |
| disulfophenyl)-2H-tetrazolium, monosodium salt | | | |
| WST-4: 2-Benzothiazoryl-3-(4-carboxy-2-methoxyphenyl)- | Dye | Dojindo | W203 |
| 5-[4-(2-sulfoethylcarbamoyl)phenyl]-2H-tetrazolium | | | |
| WST-5: 2,2′-Dibenzothiazolyl-5,5′-bis[4-di(2- | Dye | Dojindo | W204 |
| sulfoethyl)carbamoylphenyl]-3,3′-(3,3′-dimethoxy 4,4′- | | | |
| biphenylene)ditetrazolium, disodium salt | | | |
| WST-8: 5-(2,4-disulfophenyl)-3-(2-methoxy-4-nitrophenyl)- | Dye | Cayman | 18721 - 100 |
| 2-(4-nitrophenyl)-2H-tetrazolium, inner salt, monosodium salt | | | mg |
| WST-9: 2-(4-Nitrophenyl)-5-phenyl-3-[4-(4- | Dye | Dojindo | W217 |
| sulfophenylazo)-2-sulfophenyl]-2H-tetrazolium, monosodium | | | |
| salt | | | |
| XTT: 2,3-Bis(2-methoxy-4-nitro-5-sulfophenyl)-2H- | Dye | Sigma | X4626- |
| tetrazolium-5-carboxanilide inner salt | | | 100 MG |
| Rotenone | — | Sigma | R8875-1 G |

First, UV absorbance analysis was carried out. Different tetrazolium salts (2 mM) were added to an assay buffer containing 2 mM NADH, 4 mM NAD, 5 mM MgCl2, 0.2 mM 1-methoxy 5-methylphenazinium methyl sulfate (1-methoxy PMS), 15 mM malate, 5 units of malate dehydrogenase, and 5 μg diaphorase. The reactions were performed at room temperature, and absorbance was taken every hour.

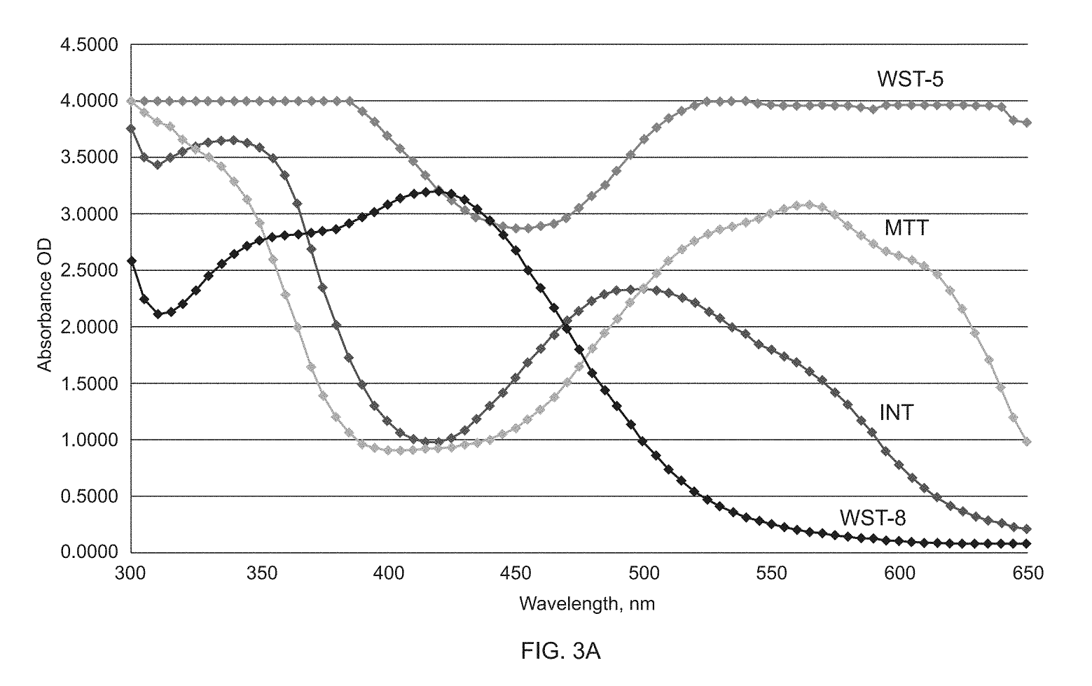

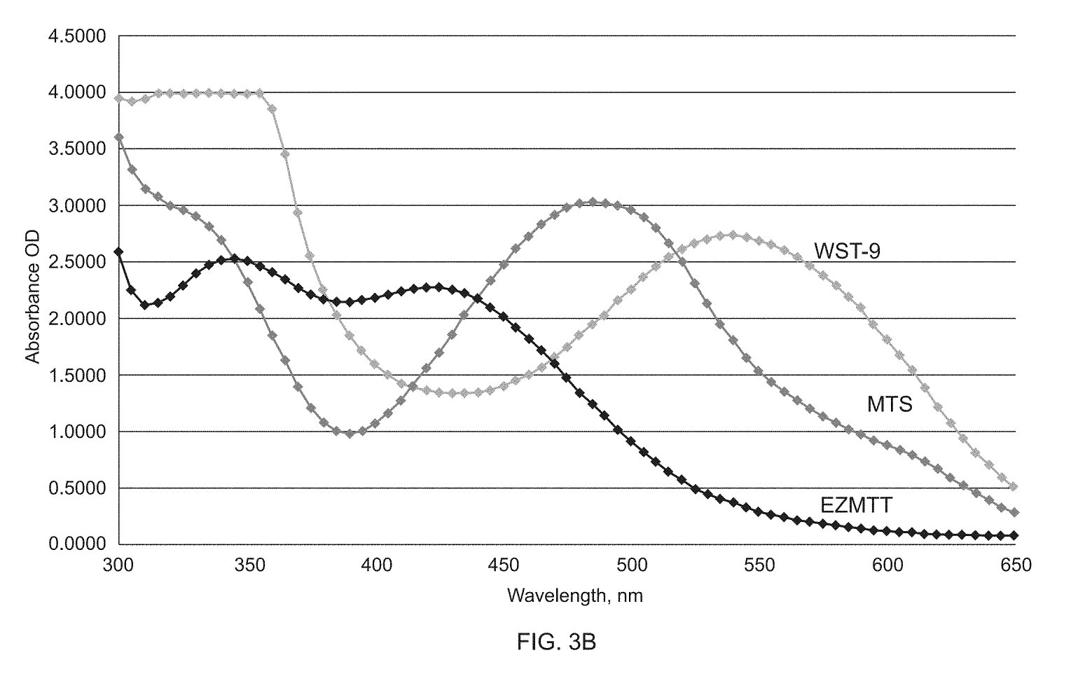

As shown in TABLE 6, each tetrazolium salt produced formazan products with different colors and distinctive absorbance wavelength (nM). MTT, Nitro-TB, WST-9 and INT form precipitates in the presence of 1-methoxy PMS. WST-1, 4, 5 and 8 form water-soluble formazan products. As shown in FIGS. 3A and 3B, each tetrazolium salt generated its own distinctive pattern of absorbance at different wavelength in the reaction buffer containing disphorase.

TABLE 6

| | | | | Wave |
|---|---|---|---|---|
| | | | | length |
| | | | Wave length | (nM) in |
| | | Tetra- | (nM) in | presence of |
| | | zolium | presence of | 1-methoxy |
| CAS # | Structure | dye | Diaphorase | PMS |
| 150849- 52-8 | 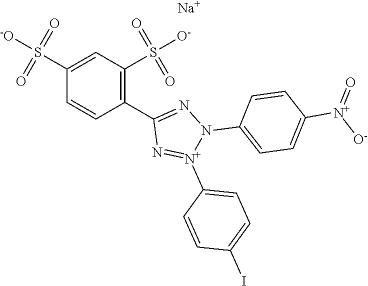 | WST-1 | 440 | 440 |
| 178925- 54-7 | 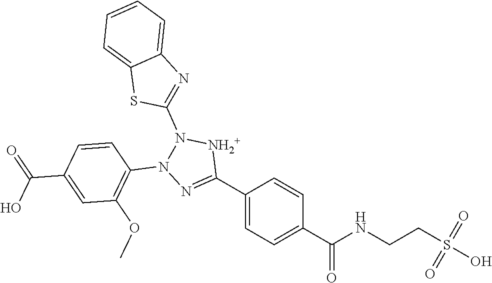 [SMILES: COc1cc(C(=O)O)ccc1N1N=C(c2ccc(C(=O)NCCS(=O)(=O)O)cc2)[NH2+]N1c1nc2ccccc2s1] | WST-4 | 565 | 565 |
| 178925- 55-8 | 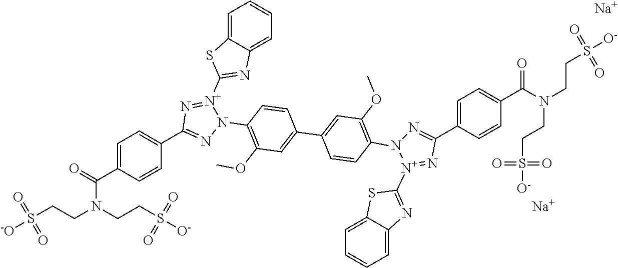 | WST-5 | 570 | 570 |
| 193149- 74-5 | 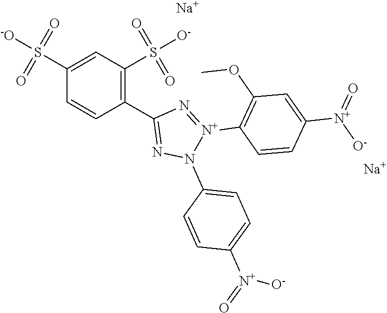 | WST-8 | 460 | 460 |
| 847986- 47-4 | 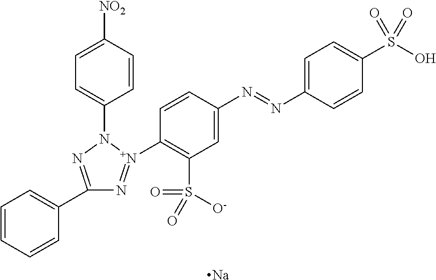 | WST-9 | 545 | 545 |
| 1997299- 51-0 | 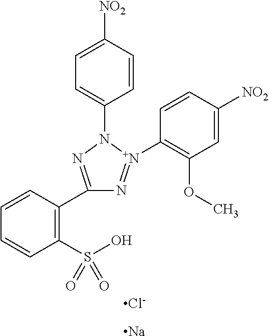 | EZMTT | 460 | 460 |
| 146-68-9 | 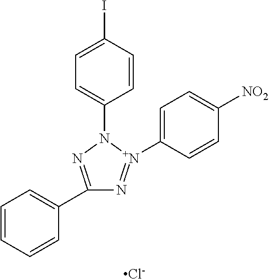 [SMILES: O=[N+]([O-])c1ccc(-[n+]2nc(-c3ccccc3)nn2-c2ccc(I)cc2)cc1 ; [Cl-]] | INT | 500 | 500 |
| 298-93-1 | 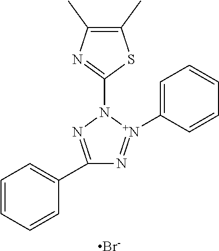 [SMILES: Cc1nc(-n2nc(-c3ccccc3)n[n+]2-c2ccccc2)sc1C ; [Br-]] | MTT | 565 | 565 |
| 138169- 43-4 | 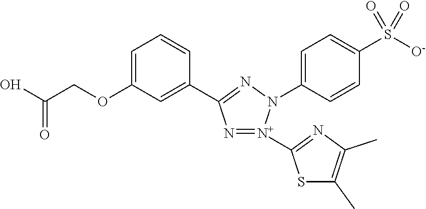 [SMILES: Cc1nc(-[n+]2nc(-c3cccc(OCC(=O)O)c3)nn2-c2ccc(S(=O)(=O)[O-])cc2)sc1C] | MTS | 485 | 485 |
| 111072- 31-2 | 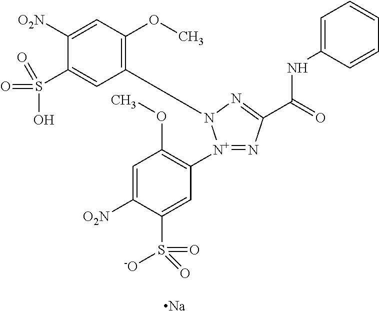 | XTT | 460 | 460 |
| 298-83-9 | 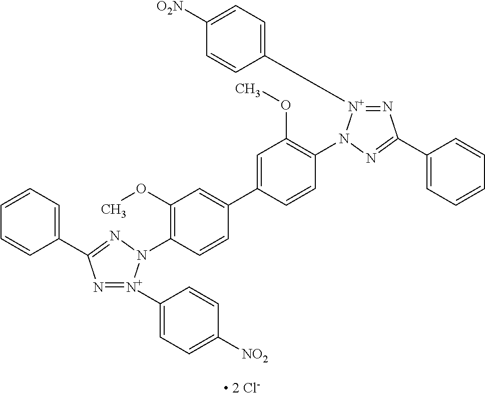 [SMILES: COc1cc(-c2ccc(-n3nc(-c4ccccc4)n[n+]3-c3ccc([N+](=O)[O-])cc3)c(OC)c2)ccc1-n1nc(-c2ccccc2)n[n+]1-c1ccc([N+](=O)[O-])cc1] | Nitro-TB | 550 | 550 |

Next, the rate of formazan formation was examined in the presence of either 1-methoxy PMS or diaphorase, or in the presence of NADP, or in the presence of NADP generation system contains malate dehydrogenase, malate and NAD+. Again, different tetrazolium salts (2 mM) were added to an assay buffer containing 2 mM NADH, 4 mM NAD, 5 mM MgCl2, 15 mM malate, 5 units of malate dehydrogenase, and 5 μg diaphorase or 0.2 mM 1-methoxy 5-methylphenazinium methyl sulfate (1-methoxy PMS). The reactions were performed at room temperature (around 22° C.). Absorbance was taken at every hour.

As shown in TABLE 7, all the tetrazolium salts were reduced to form formazan dyes immediately after adding NADH and diaphorase. WST-9 took about an hour to be completely reduced to formazan dyes.

TABLE 7
Formation of formazan dyes in the presence of NADH and diaphorase, but in the absence
of malate. The absorbance was measured at the time indicated. Each mean and standard deviation
(STDEV) were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 3.54 | 3.60 | 3.59 | 3.60 | 3.59 | 3.59 | 3.55 | 3.52 | 3.45 | 0.13 | 0.14 | 0.12 | 0.13 | 0.10 | 0.11 | 0.10 | 0.15 | 0.19 |
| WST-4 | 1.96 | 2.01 | 2.04 | 2.08 | 2.20 | 2.25 | 2.29 | 2.30 | 2.32 | 0.13 | 0.12 | 0.12 | 0.15 | 0.08 | 0.08 | 0.08 | 0.10 | 0.11 |
| WST-5 | 2.81 | 2.81 | 2.79 | 2.78 | 2.86 | 2.83 | 2.77 | 2.74 | 2.66 | 0.18 | 0.19 | 0.17 | 0.16 | 0.08 | 0.17 | 0.24 | 0.35 | 0.41 |
| WST-8 | 3.54 | 3.72 | 3.77 | 3.77 | 3.63 | 3.64 | 3.66 | 3.65 | 3.67 | 0.30 | 0.25 | 0.23 | 0.24 | 0.18 | 0.13 | 0.11 | 0.17 | 0.18 |
| WST-9 | 1.48 | 2.08 | 2.08 | 2.02 | 1.97 | 1.90 | 1.90 | 1.91 | 1.84 | 0.84 | 0.32 | 0.22 | 0.21 | 0.25 | 0.23 | 0.26 | 0.31 | 0.31 |
| INT | 2.03 | 2.14 | 2.13 | 1.99 | 2.07 | 2.04 | 2.07 | 2.00 | 2.10 | 0.03 | 0.07 | 0.05 | 0.11 | 0.02 | 0.03 | 0.14 | 0.00 | 0.07 |
| XTT | 2.22 | 2.35 | 2.36 | 2.32 | 2.40 | 2.39 | 2.45 | 2.45 | 2.46 | 0.20 | 0.07 | 0.08 | 0.11 | 0.07 | 0.06 | 0.09 | 0.06 | 0.03 |
| Nitro-TB | 1.49 | 1.71 | 1.73 | 1.71 | 1.74 | 1.86 | 1.83 | 1.85 | 1.82 | 0.25 | 0.05 | 0.07 | 0.10 | 0.08 | 0.01 | 0.07 | 0.02 | 0.12 |
| MTS | 3.21 | 3.24 | 3.22 | 3.20 | 3.27 | 3.29 | 3.28 | 3.28 | 3.28 | 0.07 | 0.08 | 0.11 | 0.10 | 0.01 | 0.04 | 0.08 | 0.06 | 0.07 |
| MTT | 2.00 | 2.04 | 1.94 | 1.77 | 1.77 | 1.62 | 1.57 | 1.55 | 1.43 | 0.10 | 0.07 | 0.07 | 0.07 | 0.01 | 0.00 | 0.06 | 0.09 | 0.07 |

As shown in TABLE 8, all the tetrazolium salts were reduced to form formazan dyes immediately after adding NADH and diaphorase as observed in TABLE 7. Again, WST-9 took about an hour to be completely reduced to formazan dyes. In the presence of malate, a lower level of WST-1 was reduced to formazan dyes.

TABLE 8
Formation of formazan dyes in the presence of NADH, diaphorase, and malate. The
absorbance was measured at the time indicated. Each mean and STDEV were derived from three
experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 2.63 | 2.60 | 2.59 | 2.57 | 2.55 | 2.52 | 2.48 | 2.44 | 2.38 | 0.25 | 0.09 | 0.10 | 0.12 | 0.17 | 0.21 | 0.26 | 0.30 | 0.39 |
| WST-4 | 1.92 | 1.91 | 1.91 | 1.89 | 1.97 | 1.94 | 1.92 | 1.88 | 1.83 | 0.14 | 0.14 | 0.15 | 0.16 | 0.11 | 0.15 | 0.20 | 0.25 | 0.32 |
| WST-5 | 2.62 | 2.48 | 2.44 | 2.41 | 2.45 | 2.41 | 2.36 | 2.31 | 2.26 | 0.12 | 0.09 | 0.12 | 0.16 | 0.18 | 0.24 | 0.30 | 0.37 | 0.44 |
| WST-8 | 3.06 | 3.36 | 3.47 | 3.53 | 3.50 | 3.54 | 3.54 | 3.51 | 3.45 | 0.15 | 0.17 | 0.17 | 0.17 | 0.17 | 0.22 | 0.30 | 0.39 | 0.50 |
| WST-9 | 0.85 | 1.34 | 1.41 | 1.38 | 1.39 | 1.34 | 1.30 | 1.28 | 1.24 | 0.72 | 0.38 | 0.23 | 0.22 | 0.26 | 0.24 | 0.25 | 0.26 | 0.31 |
| INT | 1.78 | 1.80 | 1.80 | 1.81 | 1.93 | 1.99 | 2.03 | 2.08 | 2.12 | 0.05 | 0.04 | 0.07 | 0.08 | 0.01 | 0.02 | 0.01 | 0.01 | 0.02 |
| XTT | 1.95 | 2.20 | 2.18 | 2.17 | 2.23 | 2.28 | 2.33 | 2.41 | 2.45 | 0.41 | 0.09 | 0.09 | 0.10 | 0.03 | 0.02 | 0.03 | 0.01 | 0.02 |
| Nitro-TB | 1.03 | 1.15 | 1.16 | 1.17 | 1.22 | 1.27 | 1.33 | 1.37 | 1.42 | 0.19 | 0.04 | 0.04 | 0.08 | 0.00 | 0.00 | 0.01 | 0.00 | 0.02 |
| MTS | 3.01 | 3.01 | 3.00 | 3.00 | 3.07 | 3.09 | 3.17 | 3.08 | 3.20 | 0.10 | 0.08 | 0.08 | 0.10 | 0.18 | 0.14 | 0.10 | 0.17 | 0.29 |
| MTT | 1.70 | 1.71 | 1.68 | 1.64 | 1.79 | 1.77 | 1.73 | 1.68 | 1.64 | 0.10 | 0.12 | 0.12 | 0.17 | 0.02 | 0.02 | 0.01 | 0.00 | 0.00 |

TABLE 9 showed that malate dehydrogenase may reduce some tetrazolium dyes in the absence of substrate malate. WST-1, 4, 5, 8 and 9 were partially reduced in the presence of malate dehydrogenase and diaphorase, while INT, XTT, Nitro-TB, MTS and MTT remained largely in oxidized forms.

TABLE 9 Formation of formazan dyes in the presence of Malate dehydrogenase and diaphorase, but in the absence of malate. The absorbance was measured at the time indicated. Each mean and STDEV were derived from three experiments.

TABLE 9
Formation of formazan dyes in the presence of Malate dehydrogenase and diaphorase,
but in the absence of malate. The absorbance was measured at the time indicated. Each mean and
STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.76 | 1.13 | 1.28 | 1.38 | 1.47 | 1.60 | 1.71 | 1.78 | 1.75 | 0.16 | 0.10 | 0.11 | 0.05 | 0.08 | 0.11 | 0.08 | 0.07 | 0.07 |
| WST-4 | 0.51 | 0.83 | 0.99 | 1.06 | 1.17 | 1.24 | 1.35 | 1.44 | 1.47 | 0.12 | 0.08 | 0.05 | 0.04 | 0.03 | 0.04 | 0.07 | 0.09 | 0.14 |
| WST-5 | 0.83 | 1.35 | 1.58 | 1.74 | 1.86 | 2.06 | 2.24 | 2.33 | 2.33 | 0.27 | 0.19 | 0.16 | 0.14 | 0.19 | 0.23 | 0.27 | 0.33 | 0.42 |
| WST-8 | 1.13 | 1.84 | 2.15 | 2.32 | 2.41 | 2.62 | 2.72 | 2.80 | 2.89 | 0.32 | 0.17 | 0.10 | 0.14 | 0.17 | 0.08 | 0.09 | 0.16 | 0.15 |
| WST-9 | 0.39 | 0.60 | 0.66 | 0.67 | 0.69 | 0.72 | 0.77 | 0.77 | 0.81 | 0.12 | 0.10 | 0.09 | 0.08 | 0.07 | 0.04 | 0.04 | 0.08 | 0.05 |
| INT | 0.15 | 0.22 | 0.22 | 0.20 | 0.21 | 0.14 | 0.11 | 0.12 | 0.12 | 0.06 | 0.07 | 0.06 | 0.08 | 0.11 | 0.05 | 0.01 | 0.01 | 0.02 |
| XTT | 0.39 | 0.44 | 0.46 | 0.50 | 0.50 | 0.54 | 0.57 | 0.58 | 0.66 | 0.06 | 0.08 | 0.07 | 0.06 | 0.11 | 0.09 | 0.08 | 0.15 | 0.02 |
| Nitro-TB | 0.15 | 0.20 | 0.23 | 0.25 | 0.36 | 0.39 | 0.40 | 0.41 | 0.42 | 0.05 | 0.07 | 0.07 | 0.07 | 0.03 | 0.04 | 0.04 | 0.04 | 0.05 |
| MTS | 0.17 | 0.24 | 0.22 | 0.25 | 0.21 | 0.22 | 0.23 | 0.26 | 0.25 | 0.03 | 0.06 | 0.07 | 0.03 | 0.04 | 0.02 | 0.01 | 0.00 | 0.02 |
| MTT | 0.21 | 0.29 | 0.24 | 0.19 | 0.15 | 0.17 | 0.20 | 0.18 | 0.21 | 0.06 | 0.05 | 0.08 | 0.06 | 0.00 | 0.03 | 0.12 | 0.02 | 0.02 |

If malate was added to the system as shown in TABLE 10, INT, MTT, XTT, Nitro-TB and MTS were converted to reduced formazan dyes in a time-dependent manner WST-1, 4, 5 and 8 were also converted to reduced formazan dyes in a time-dependent fashion. However, WST-9 remained largely as an oxidized salt.

TABLE 10
Formation of formazan dyes in the presence of Malate dehydrogenase, diaphorase, and malate. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.84 | 1.15 | 1.31 | 1.44 | 1.53 | 1.69 | 1.81 | 1.90 | 1.96 | 0.14 | 0.11 | 0.11 | 0.12 | 0.16 | 0.20 | 0.25 | 0.29 | 0.34 |
| WST-4 | 0.51 | 0.81 | 0.99 | 1.13 | 1.25 | 1.43 | 1.57 | 1.68 | 1.75 | 0.16 | 0.12 | 0.11 | 0.11 | 0.14 | 0.16 | 0.18 | 0.22 | 0.28 |
| WST-5 | 0.75 | 1.32 | 1.67 | 1.93 | 2.04 | 2.20 | 2.34 | 2.49 | 2.60 | 0.30 | 0.27 | 0.28 | 0.28 | 0.26 | 0.30 | 0.43 | 0.52 | 0.55 |
| WST-8 | 1.13 | 1.93 | 2.36 | 2.65 | 2.86 | 3.12 | 3.26 | 3.30 | 3.28 | 0.41 | 0.31 | 0.27 | 0.25 | 0.29 | 0.27 | 0.28 | 0.31 | 0.37 |
| WST-9 | 0.36 | 0.59 | 0.71 | 0.80 | 0.86 | 0.97 | 1.04 | 1.10 | 1.13 | 0.20 | 0.17 | 0.18 | 0.19 | 0.26 | 0.28 | 0.29 | 0.30 | 0.32 |
| INT | 0.78 | 1.26 | 1.57 | 1.81 | 2.24 | 2.71 | 3.11 | 3.44 | 3.66 | 0.22 | 0.05 | 0.07 | 0.13 | 0.00 | 0.02 | 0.00 | 0.01 | 0.04 |
| XTT | 1.21 | 1.98 | 2.41 | 2.71 | 3.02 | 3.54 | 3.86 | 3.90 | 3.80 | 0.47 | 0.23 | 0.16 | 0.15 | 0.04 | 0.04 | 0.05 | 0.08 | 0.09 |
| Nitro-TB | 0.56 | 0.88 | 1.08 | 1.23 | 1.46 | 1.73 | 1.95 | 2.16 | 2.35 | 0.18 | 0.07 | 0.05 | 0.10 | 0.00 | 0.02 | 0.05 | 0.06 | 0.06 |
| MTS | 1.72 | 2.67 | 2.93 | 3.12 | 3.48 | 3.67 | 3.73 | 3.74 | 3.71 | 0.61 | 0.04 | 0.13 | 0.15 | 0.00 | 0.04 | 0.05 | 0.01 | 0.00 |
| MTT | 0.75 | 1.29 | 1.61 | 1.81 | 2.25 | 2.67 | 2.94 | 3.07 | 3.10 | 0.26 | 0.07 | 0.05 | 0.15 | 0.01 | 0.02 | 0.05 | 0.04 | 0.03 |

Part of the oral lavage samples from the high bleeder group, collected from Example 1, were pooled and used for the enzymatic assays. The pooled oral lavage samples, containing various enzymes and proteins, were added to the assay buffer, which contained 2 mM NADH, 4 mM NAD, 5 mM MgCl2, 15 mM malate, 5 units of malate dehydrogenase, and 5 μg diaphorase or 0.2 mM 1-methoxy 5-methylphenazinium methyl sulfate (1-methoxy PMS). As shown in TABLE 11, WST-1, 4, 5 and 8 were partially reduced to formazan dyes. Similarly, INT, XTT, Nitro-TB, MTS and MTT were also changed to formazan dyes in a significant amount. It should also be noted that the oral lavage also contains a small amount of malate.

TABLE 11
Formation of formazan dyes in the presence of oral lavage and diaphorase, but not malate. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 1.01 | 1.24 | 1.28 | 1.30 | 1.29 | 1.42 | 1.34 | 1.31 | 1.42 | 0.14 | 0.09 | 0.10 | 0.09 | 0.22 | 0.08 | 0.18 | 0.33 | 0.25 |
| WST-4 | 0.55 | 0.74 | 0.76 | 0.80 | 0.78 | 0.88 | 0.90 | 0.87 | 0.93 | 0.08 | 0.07 | 0.12 | 0.07 | 0.09 | 0.13 | 0.12 | 0.19 | 0.16 |
| WST-5 | 0.60 | 0.72 | 0.77 | 0.80 | 0.78 | 0.83 | 0.84 | 0.81 | 0.80 | 0.04 | 0.05 | 0.09 | 0.10 | 0.11 | 0.17 | 0.16 | 0.19 | 0.24 |
| WST-8 | 0.59 | 0.83 | 0.87 | 0.84 | 0.82 | 0.88 | 1.00 | 1.00 | 1.01 | 0.06 | 0.09 | 0.11 | 0.10 | 0.09 | 0.13 | 0.12 | 0.20 | 0.27 |
| WST-9 | 0.45 | 0.63 | 0.64 | 0.67 | 0.59 | 0.62 | 0.71 | 0.64 | 0.67 | 0.06 | 0.09 | 0.12 | 0.15 | 0.13 | 0.20 | 0.14 | 0.22 | 0.18 |
| INT | 0.64 | 0.92 | 1.04 | 1.01 | 1.18 | 1.32 | 1.35 | 1.43 | 1.48 | 0.08 | 0.06 | 0.09 | 0.29 | 0.09 | 0.10 | 0.16 | 0.18 | 0.09 |
| XTT | 0.80 | 0.98 | 1.04 | 0.99 | 1.18 | 1.24 | 1.26 | 1.27 | 1.28 | 0.05 | 0.08 | 0.09 | 0.13 | 0.10 | 0.05 | 0.07 | 0.12 | 0.07 |
| Nitro-TB | 0.56 | 0.79 | 0.85 | 0.81 | 0.88 | 0.87 | 0.88 | 0.86 | 0.87 | 0.10 | 0.15 | 0.21 | 0.22 | 0.12 | 0.09 | 0.14 | 0.10 | 0.12 |
| MTS | 0.70 | 0.86 | 0.91 | 1.00 | 1.13 | 1.18 | 1.21 | 1.25 | 1.27 | 0.08 | 0.19 | 0.26 | 0.23 | 0.28 | 0.32 | 0.32 | 0.33 | 0.25 |
| MTT | 0.97 | 1.29 | 1.49 | 1.52 | 1.69 | 1.82 | 1.76 | 1.91 | 1.94 | 0.18 | 0.19 | 0.21 | 0.23 | 0.15 | 0.17 | 0.37 | 0.34 | 0.22 |

Interestingly, addition of malate in the assay system increased the rate of formazan formation in the presence of oral lavage, even though the increase was small, as shown in TABLE 12.

TABLE 12
Formation of formazan dyes in the presence of oral lavage, malate and diaphorase. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2h | 3 h | 4 h | 6 h | 8 h | 10 h | 12 h | 0 h | 1 h | 2 h | 3 h | 4 h | 6 h | 8 h | 10 h | 12 h |
| WST-1 | 0.88 | 1.19 | 1.19 | 1.23 | 1.39 | 1.31 | 1.52 | 1.61 | 1.75 | 0.18 | 0.09 | 0.08 | 0.15 | 0.11 | 0.16 | 0.27 | 0.37 | 0.37 |
| WST-4 | 0.44 | 0.56 | 0.63 | 0.67 | 0.77 | 0.83 | 0.89 | 0.96 | 0.95 | 0.09 | 0.11 | 0.11 | 0.10 | 0.10 | 0.07 | 0.17 | 0.23 | 0.21 |
| WST-5 | 0.57 | 0.74 | 0.80 | 0.75 | 0.67 | 0.85 | 0.90 | 1.02 | 1.08 | 0.13 | 0.05 | 0.11 | 0.17 | 0.09 | 0.23 | 0.09 | 0.18 | 0.25 |
| WST-8 | 0.51 | 0.70 | 0.80 | 0.84 | 0.85 | 0.91 | 1.05 | 1.09 | 1.33 | 0.12 | 0.07 | 0.14 | 0.16 | 0.15 | 0.21 | 0.19 | 0.28 | 0.26 |
| WST-9 | 0.46 | 0.56 | 0.57 | 0.61 | 0.65 | 0.58 | 0.63 | 0.64 | 0.78 | 0.11 | 0.13 | 0.13 | 0.16 | 0.20 | 0.23 | 0.20 | 0.19 | 0.16 |
| INT | 0.52 | 0.81 | 0.92 | 0.96 | 1.10 | 1.20 | 1.44 | 1.84 | 2.37 | 0.07 | 0.12 | 0.17 | 0.35 | 0.11 | 0.13 | 0.03 | 0.08 | 0.09 |
| XTT | 0.75 | 0.88 | 0.94 | 0.98 | 1.19 | 1.41 | 1.73 | 1.90 | 2.08 | 0.10 | 0.09 | 0.11 | 0.17 | 0.03 | 0.16 | 0.03 | 0.02 | 0.14 |
| Nitro-TB | 0.52 | 0.75 | 0.80 | 0.75 | 0.88 | 1.00 | 1.09 | 1.20 | 1.30 | 0.10 | 0.07 | 0.08 | 0.13 | 0.05 | 0.03 | 0.01 | 0.08 | 0.09 |
| MTS | 0.68 | 0.93 | 1.07 | 1.14 | 1.34 | 1.56 | 1.84 | 2.10 | 2.27 | 0.15 | 0.28 | 0.30 | 0.39 | 0.28 | 0.38 | 0.52 | 0.66 | 0.59 |
| MTT | 0.77 | 1.15 | 1.36 | 1.52 | 1.65 | 1.94 | 2.11 | 2.41 | 2.72 | 0.16 | 0.23 | 0.28 | 0.27 | 0.27 | 0.29 | 0.27 | 0.14 | 0.08 |

If NADH and malate dehydrogenase are not added, diaphorase could not convert tetrazolium salts into formazan dyes in the absence of NADH as shown in TABLE 13 and TABLE 14.

TABLE 13
Formation of formazan dyes in the presence of only diaphorase, but not malate. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.41 | 0.60 | 0.66 | 0.69 | 0.70 | 0.71 | 0.73 | 0.69 | 0.67 | 0.16 | 0.10 | 0.09 | 0.10 | 0.03 | 0.08 | 0.09 | 0.10 | 0.14 |
| WST-4 | 0.22 | 0.35 | 0.40 | 0.40 | 0.38 | 0.30 | 0.32 | 0.36 | 0.39 | 0.06 | 0.03 | 0.03 | 0.05 | 0.06 | 0.06 | 0.10 | 0.08 | 0.14 |
| WST-5 | 0.25 | 0.30 | 0.35 | 0.32 | 0.32 | 0.38 | 0.41 | 0.38 | 0.36 | 0.07 | 0.02 | 0.05 | 0.07 | 0.05 | 0.11 | 0.14 | 0.11 | 0.12 |
| WST-8 | 0.22 | 0.38 | 0.46 | 0.48 | 0.43 | 0.46 | 0.44 | 0.44 | 0.47 | 0.09 | 0.06 | 0.06 | 0.06 | 0.07 | 0.10 | 0.18 | 0.16 | 0.10 |
| WST-9 | 0.19 | 0.28 | 0.29 | 0.30 | 0.29 | 0.28 | 0.27 | 0.31 | 0.27 | 0.06 | 0.04 | 0.03 | 0.06 | 0.08 | 0.14 | 0.11 | 0.06 | 0.08 |
| INT | 0.14 | 0.25 | 0.23 | 0.24 | 0.24 | 0.25 | 0.23 | 0.20 | 0.24 | 0.04 | 0.03 | 0.02 | 0.10 | 0.09 | 0.00 | 0.01 | 0.09 | 0.12 |
| XTT | 0.41 | 0.49 | 0.52 | 0.54 | 0.54 | 0.60 | 0.63 | 0.64 | 0.67 | 0.03 | 0.04 | 0.05 | 0.11 | 0.11 | 0.07 | 0.06 | 0.07 | 0.11 |
| Nitro-TB | 0.15 | 0.19 | 0.21 | 0.21 | 0.31 | 0.33 | 0.34 | 0.36 | 0.37 | 0.04 | 0.05 | 0.05 | 0.07 | 0.03 | 0.03 | 0.03 | 0.03 | 0.03 |
| MTS | 0.16 | 0.25 | 0.26 | 0.22 | 0.17 | 0.20 | 0.16 | 0.13 | 0.14 | 0.02 | 0.05 | 0.05 | 0.03 | 0.02 | 0.11 | 0.05 | 0.01 | 0.01 |
| MTT | 0.21 | 0.27 | 0.23 | 0.24 | 0.17 | 0.14 | 0.23 | 0.22 | 0.24 | 0.06 | 0.05 | 0.09 | 0.04 | 0.01 | 0.02 | 0.14 | 0.01 | 0.02 |

TABLE 14
Formation of formazan dyes in the presence of only diaphorase and malate. The absorbance was
measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.20 | 0.22 | 0.23 | 0.24 | 0.24 | 0.25 | 0.26 | 0.27 | 0.27 | 0.02 | 0.02 | 0.02 | 0.01 | 0.01 | 0.02 | 0.02 | 0.03 | 0.04 |
| WST-4 | 0.08 | 0.10 | 0.12 | 0.14 | 0.16 | 0.19 | 0.23 | 0.26 | 0.27 | 0.02 | 0.02 | 0.02 | 0.02 | 0.02 | 0.02 | 0.02 | 0.02 | 0.03 |
| WST-5 | 0.09 | 0.12 | 0.15 | 0.18 | 0.21 | 0.23 | 0.26 | 0.29 | 0.32 | 0.03 | 0.02 | 0.03 | 0.02 | 0.03 | 0.04 | 0.04 | 0.04 | 0.04 |
| WST-8 | 0.10 | 0.14 | 0.16 | 0.19 | 0.21 | 0.26 | 0.31 | 0.35 | 0.38 | 0.03 | 0.02 | 0.02 | 0.02 | 0.02 | 0.02 | 0.01 | 0.02 | 0.01 |
| WST-9 | 0.07 | 0.08 | 0.09 | 0.10 | 0.12 | 0.14 | 0.15 | 0.16 | 0.18 | 0.02 | 0.02 | 0.02 | 0.02 | 0.01 | 0.01 | 0.01 | 0.02 | 0.02 |
| INT | 0.08 | 0.10 | 0.12 | 0.13 | 0.18 | 0.23 | 0.25 | 0.28 | 0.33 | 0.02 | 0.02 | 0.02 | 0.03 | 0.00 | 0.00 | 0.02 | 0.06 | 0.11 |
| XTT | 0.39 | 0.45 | 0.48 | 0.53 | 0.52 | 0.57 | 0.63 | 0.69 | 0.73 | 0.07 | 0.07 | 0.07 | 0.06 | 0.04 | 0.06 | 0.05 | 0.07 | 0.08 |
| Nitro-TB | 0.07 | 0.08 | 0.09 | 0.10 | 0.13 | 0.16 | 0.18 | 0.19 | 0.21 | 0.02 | 0.02 | 0.02 | 0.02 | 0.00 | 0.01 | 0.01 | 0.01 | 0.00 |
| MTS | 0.16 | 0.22 | 0.27 | 0.32 | 0.46 | 0.57 | 0.68 | 0.79 | 0.93 | 0.06 | 0.05 | 0.04 | 0.04 | 0.01 | 0.04 | 0.07 | 0.08 | 0.00 |
| MTT | 0.08 | 0.11 | 0.13 | 0.14 | 0.20 | 0.24 | 0.25 | 0.28 | 0.31 | 0.01 | 0.02 | 0.02 | 0.03 | 0.01 | 0.01 | 0.01 | 0.01 | 0.01 |

Next examined was the effect of 1-methoxy PMS on formation of formazan dyes in the presence of NADH. WST-8 was converted to formazan dyes quickly in the presence of 1-methoxy PMS in the absence of malate (TABLE 15) or in the presence of malate (TABLE 16). MTT, Nitro-TB and INT formed precipitates when both 1-methoxy PMS and NADH were added in the absence of malate (TABLE 15) or in the presence of malate (TABLE 16). WST-9 also formed precipitated products.

TABLE 15
Formation of formazan dyes in the presence of NADH and 1-methoxy PMS, but in the absence of malate. The
absorbance was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 2.52 | 2.71 | 2.59 | 2.80 | | | | | | 0.33 | 0.13 | 0.11 | 0.22 | | | | | |
| WST-4 | 1.52 | 1.57 | 1.57 | 1.64 | | | | | | 0.05 | 0.06 | 0.08 | 0.07 | | | | | |
| WST-5 | 2.12 | 2.14 | 2.18 | 2.22 | | | | | | 0.08 | 0.06 | 0.11 | 0.15 | | | | | |
| WST-8 | 3.65 | 3.66 | 3.66 | 3.67 | | | | | | 0.28 | 0.31 | 0.33 | 0.33 | | | | | |
| WST-9 | 1.36 | 1.47 | 1.44 | 1.33 | | | | | | 0.18 | 0.08 | 0.07 | 0.05 | | | | | |
| INT | 0.96 | 0.91 | 0.95 | 0.94 | 0.66 | 0.77 | 0.75 | 0.78 | 0.98 | 0.26 | 0.19 | 0.25 | 0.13 | 0.16 | 0.11 | 0.14 | 0.23 | 0.33 |
| XTT | 1.87 | 1.98 | 2.08 | 2.12 | 2.06 | 2.20 | 2.22 | 2.33 | 2.32 | 0.16 | 0.18 | 0.15 | 0.13 | 0.09 | 0.06 | 0.05 | 0.06 | 0.04 |
| Nitro-TB | 0.99 | 1.56 | 1.76 | 1.66 | 2.13 | 1.50 | 1.30 | 1.79 | 1.55 | 0.28 | 0.17 | 0.57 | 0.52 | 0.25 | 0.04 | 0.09 | 0.05 | 0.18 |
| MTS | 2.31 | 2.55 | 2.60 | 2.59 | 2.63 | 2.65 | 2.69 | 2.67 | 2.65 | 0.41 | 0.41 | 0.34 | 0.37 | 0.01 | 0.05 | 0.06 | 0.05 | 0.02 |
| MTT | 0.91 | 0.69 | 0.70 | 0.78 | 0.91 | 0.76 | 0.69 | 0.73 | 0.68 | 0.21 | 0.15 | 0.11 | 0.20 | 0.14 | 0.18 | 0.17 | 0.15 | 0.02 |

TABLE 16
Formation of formazan dyes in the presence of NADH, malate and 1-methoxy PMS. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 1.83 | 1.81 | 1.79 | 1.79 | | | | | | 0.09 | 0.09 | 0.08 | 0.09 | | | | | |
| WST-4 | 1.32 | 1.31 | 1.29 | 1.30 | | | | | | 0.04 | 0.04 | 0.04 | 0.04 | | | | | |
| WST-5 | 1.84 | 1.84 | 1.84 | 1.84 | | | | | | 0.05 | 0.05 | 0.05 | 0.03 | | | | | |
| WST-8 | 3.37 | 3.42 | 3.41 | 3.42 | | | | | | 0.12 | 0.04 | 0.03 | 0.06 | | | | | |
| WST-9 | 0.78 | 0.83 | 0.80 | 0.81 | | | | | | 0.14 | 0.03 | 0.05 | 0.04 | | | | | |
| INT | 0.81 | 0.83 | 0.78 | 0.78 | 0.47 | 0.52 | 0.70 | 0.72 | 0.94 | 0.23 | 0.35 | 0.44 | 0.44 | 0.12 | 0.08 | 0.46 | 0.23 | 0.19 |
| XTT | 1.51 | 1.56 | 1.61 | 1.67 | 1.56 | 1.80 | 1.94 | 2.02 | 2.15 | 0.20 | 0.18 | 0.19 | 0.17 | 0.05 | 0.04 | 0.07 | 0.07 | 0.19 |
| Nitro-TB | 0.61 | 1.02 | 1.38 | 1.22 | 1.65 | 1.63 | 1.20 | 1.15 | 1.13 | 0.10 | 0.20 | 0.43 | 0.42 | 0.37 | 0.77 | 0.13 | 0.47 | 0.34 |
| MTS | 2.33 | 2.41 | 2.44 | 2.47 | 2.27 | 2.38 | 2.41 | 2.45 | 2.40 | 0.27 | 0.23 | 0.23 | 0.20 | 0.10 | 0.13 | 0.15 | 0.23 | 0.12 |
| MTT | 0.61 | 0.45 | 0.48 | 0.46 | 0.30 | 0.53 | 0.39 | 0.60 | 0.53 | 0.17 | 0.11 | 0.15 | 0.19 | 0.02 | 0.29 | 0.06 | 0.13 | 0.25 |

Malate dehydrogenase can oxidize malate and reduce NAD+ to NADH+H at the same time. Without malate in the assay medium, the rate and extent of tetrazolium reduction did not change as shown in TABLE 17. It is worth noting that malate dehydrogenase alone did not catalyze the reduction of WST-1, 4, 5, and 8 even in the presence of electron coupling reagent 1-methoxy PMS (TABLE 17). However, the combination of malate dehydrogenase and diaphorase was able to catalyze the reduction of WST-1, 4, 5 and 8 as shown in TABLE 10.

TABLE 17
Formation of formazan dyes in the presence of Malate dehydrogenase and 1-Methoxy PMS, but in the absence of malate.
The absorbance was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.51 | 0.71 | 0.59 | 0.90 | | | | | | 0.12 | 0.10 | 0.30 | 0.03 | | | | | |
| WST-4 | 0.27 | 0.29 | 0.31 | 0.38 | | | | | | 0.02 | 0.08 | 0.09 | 0.06 | | | | | |
| WST-5 | 0.24 | 0.37 | 0.41 | 0.43 | | | | | | 0.03 | 0.11 | 0.10 | 0.10 | | | | | |
| WST-8 | 0.35 | 0.47 | 0.47 | 0.47 | | | | | | 0.06 | 0.07 | 0.09 | 0.07 | | | | | |
| WST-9 | 0.33 | 0.45 | 0.45 | 0.54 | | | | | | 0.05 | 0.04 | 0.12 | 0.08 | | | | | |
| INT | 0.33 | 0.43 | 0.42 | 0.42 | 0.37 | 0.38 | 0.43 | 0.45 | 0.52 | 0.06 | 0.06 | 0.05 | 0.06 | 0.07 | 0.03 | 0.03 | 0.07 | 0.04 |
| XTT | 0.61 | 0.72 | 0.80 | 0.82 | 0.84 | 0.85 | 0.85 | 0.91 | 0.93 | 0.14 | 0.06 | 0.03 | 0.01 | 0.00 | 0.01 | 0.08 | 0.11 | 0.12 |
| Nitro-TB | 0.31 | 0.40 | 0.39 | 0.35 | 0.35 | 0.36 | 0.32 | 0.41 | 0.40 | 0.06 | 0.05 | 0.06 | 0.04 | 0.01 | 0.04 | 0.01 | 0.10 | 0.08 |
| MTS | 0.34 | 0.36 | 0.33 | 0.33 | 0.41 | 0.48 | 0.48 | 0.52 | 0.51 | 0.07 | 0.12 | 0.07 | 0.09 | 0.01 | 0.08 | 0.05 | 0.00 | 0.04 |
| MTT | 0.41 | 0.46 | 0.44 | 0.40 | 0.36 | 0.37 | 0.43 | 0.46 | 0.53 | 0.11 | 0.05 | 0.04 | 0.03 | 0.05 | 0.04 | 0.01 | 0.04 | 0.02 |

In the presence of malate, malate dehydrogenase produced NADH+H by oxidizing malate. The rate and extent of tetrazolium reduction were increased as shown in TABLE 18.

TABLE 18
Formation of formazan dyes in the presence of Malate dehydrogenase, malate and 1-Methoxy PMS. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.51 | 0.64 | 0.72 | 0.79 | | | | | | 0.12 | 0.07 | 0.05 | 0.05 | | | | | |
| WST-4 | 0.61 | 1.03 | 1.28 | 1.48 | | | | | | 0.27 | 0.15 | 0.12 | 0.09 | | | | | |
| WST-5 | 0.83 | 1.50 | 1.88 | 2.18 | | | | | | 0.44 | 0.25 | 0.19 | 0.15 | | | | | |
| WST-8 | 1.38 | 2.47 | 2.99 | 3.41 | | | | | | 0.71 | 0.30 | 0.15 | 0.06 | | | | | |
| WST-9 | 0.20 | 0.21 | 0.22 | 0.24 | | | | | | 0.03 | 0.02 | 0.01 | 0.02 | | | | | |
| INT | 0.68 | 0.90 | 0.86 | 0.88 | 0.76 | 0.75 | 0.65 | 0.65 | 0.55 | 0.24 | 0.05 | 0.04 | 0.07 | 0.02 | 0.04 | 0.11 | 0.13 | 0.02 |
| XTT | 2.09 | 3.28 | 3.71 | 3.96 | 4.00 | 4.00 | 4.00 | 4.00 | 4.00 | 0.87 | 0.57 | 0.35 | 0.06 | 0.00 | 0.00 | 0.00 | 0.00 | 0.00 |
| Nitro-TB | 0.51 | 0.84 | 1.29 | 1.40 | 1.21 | 1.27 | 1.25 | 1.22 | 1.33 | 0.17 | 0.15 | 0.14 | 0.24 | 0.14 | 0.04 | 0.11 | 0.05 | 0.42 |
| MTS | 2.40 | 3.33 | 3.42 | 3.38 | 3.38 | 3.27 | 3.26 | 3.18 | 3.09 | 0.90 | 0.18 | 0.03 | 0.04 | 0.04 | 0.03 | 0.02 | 0.02 | 0.09 |
| MTT | 0.88 | 0.98 | 1.25 | 1.48 | 1.47 | 1.50 | 1.46 | 1.41 | 1.38 | 0.26 | 0.06 | 0.13 | 0.10 | 0.06 | 0.06 | 0.11 | 0.10 | 0.14 |

Oral lavage contains both malate dehydrogenase and malate. Adding oral lavage alone promoted the change of tetrazolium salts into colored formazan products as shown in TABLE 19.

TABLE 19
Formation of formazan dyes in the presence of oral lavage and 1-Methoxy PMS, but in the absence of malate.
The absorbance was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 1.19 | 1.52 | 1.61 | 1.76 | | | | | | 0.34 | 0.06 | 0.10 | 0.12 | | | | | |
| WST-4 | 0.78 | 1.07 | 1.15 | 1.22 | | | | | | 0.14 | 0.05 | 0.04 | 0.02 | | | | | |
| WST-5 | 0.77 | 1.11 | 1.29 | 1.39 | | | | | | 0.12 | 0.11 | 0.13 | 0.13 | | | | | |
| WST-8 | 1.03 | 1.38 | 1.68 | 1.86 | | | | | | 0.19 | 0.13 | 0.34 | 0.37 | | | | | |
| WST-9 | 1.02 | 1.26 | 1.28 | 1.57 | | | | | | 0.23 | 0.14 | 0.27 | 0.37 | | | | | |
| INT | 0.80 | 1.27 | 1.46 | 1.55 | 1.67 | 1.98 | 1.99 | 2.21 | 2.31 | 0.21 | 0.10 | 0.07 | 0.08 | 0.03 | 0.01 | 0.00 | 0.05 | 0.03 |
| XTT | 1.14 | 1.84 | 2.15 | 2.45 | 2.79 | 3.06 | 3.19 | 3.38 | 3.32 | 0.36 | 0.19 | 0.29 | 0.39 | 0.13 | 0.16 | 0.30 | 0.10 | 0.05 |
| Nitro-TB | 0.52 | 0.58 | 0.61 | 0.63 | 0.60 | 0.63 | 0.68 | 0.73 | 0.82 | 0.04 | 0.04 | 0.05 | 0.06 | 0.13 | 0.15 | 0.12 | 0.14 | 0.06 |
| MTS | 0.87 | 1.50 | 1.82 | 2.08 | 1.94 | 2.32 | 2.51 | 2.66 | 2.74 | 0.19 | 0.13 | 0.22 | 0.34 | 0.15 | 0.20 | 0.15 | 0.11 | 0.07 |
| MTT | 0.92 | 1.34 | 1.40 | 1.60 | 1.43 | 1.51 | 1.51 | 1.44 | 1.36 | 0.19 | 0.11 | 0.13 | 0.17 | 0.07 | 0.01 | 0.12 | 0.11 | 0.06 |

When both oral lavage and substrate malate were added, the rate and extent of converting tetrazolium salts into colored formazan dyes increased as shown in TABLE 20.

TABLE 20
Formation of formazan dyes in the presence of oral lavage, malate and 1-Methoxy PMS, but in the absence of malate.
The absorbance was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 1.20 | 1.68 | 2.01 | 2.32 | | | | | | 0.36 | 0.31 | 0.34 | 0.40 | | | | | |
| WST-4 | 0.73 | 1.14 | 1.44 | 1.61 | | | | | | 0.29 | 0.23 | 0.29 | 0.33 | | | | | |
| WST-5 | 0.72 | 1.24 | 1.56 | 1.95 | | | | | | 0.24 | 0.26 | 0.34 | 0.26 | | | | | |
| WST-8 | 0.98 | 1.39 | 1.83 | 2.31 | | | | | | 0.35 | 0.31 | 0.31 | 0.37 | | | | | |
| WST-9 | 1.01 | 1.34 | 1.39 | 1.62 | | | | | | 0.35 | 0.13 | 0.12 | 0.21 | | | | | |
| INT | 0.56 | 0.87 | 1.01 | 1.04 | 0.80 | 1.04 | 1.26 | 1.53 | 1.69 | 0.17 | 0.22 | 0.33 | 0.27 | 0.14 | 0.31 | 0.53 | 0.83 | 0.62 |
| XTT | 0.90 | 1.20 | 1.54 | 1.66 | 1.83 | 1.91 | 1.92 | 1.98 | 2.59 | 0.36 | 0.35 | 0.21 | 0.37 | 0.49 | 0.22 | 0.42 | 0.10 | 0.06 |
| Nitro-TB | 0.35 | 0.37 | 0.40 | 0.43 | 0.29 | 0.29 | 0.30 | 0.30 | 0.31 | 0.10 | 0.12 | 0.14 | 0.16 | 0.01 | 0.01 | 0.01 | 0.01 | 0.01 |
| MTS | 0.78 | 1.33 | 1.69 | 1.90 | 1.87 | 2.27 | 2.60 | 2.89 | 3.22 | 0.30 | 0.16 | 0.18 | 0.22 | 0.10 | 0.14 | 0.05 | 0.00 | 0.16 |
| MTT | 0.72 | 1.20 | 1.44 | 1.56 | 1.74 | 1.57 | 1.46 | 1.44 | 1.43 | 0.22 | 0.14 | 0.15 | 0.21 | 0.06 | 0.07 | 0.13 | 0.21 | 0.12 |

1-Methoxy PMS is an electron coupling reagent. XTT and MTS appeared to slowly catalyze the conversion of tetrazolium salts into colored formazan dyes in the assay buffer containing 1-methoxy PMS, in the absence of malate (TABLE 21) or in the presence of malate (TABLE 22).

TABLE 21
Formation of formazan dyes in the presence of control buffer and 1-Methoxy PMS, but in the absence of malate.
The absorbance was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.53 | 0.74 | 0.65 | 0.88 | | | | | | 0.09 | 0.13 | 0.29 | 0.05 | | | | | |
| WST-4 | 0.30 | 0.34 | 0.43 | 0.44 | | | | | | 0.05 | 0.14 | 0.04 | 0.03 | | | | | |
| WST-5 | 0.33 | 0.45 | 0.48 | 0.55 | | | | | | 0.06 | 0.06 | 0.03 | 0.06 | | | | | |
| WST-8 | 0.44 | 0.55 | 0.49 | 0.62 | | | | | | 0.03 | 0.06 | 0.09 | 0.04 | | | | | |
| WST-9 | 0.31 | 0.38 | 0.30 | 0.43 | | | | | | 0.04 | 0.03 | 0.10 | 0.06 | | | | | |
| INT | 0.37 | 0.41 | 0.39 | 0.35 | 0.33 | 0.38 | 0.43 | 0.35 | 0.44 | 0.11 | 0.06 | 0.03 | 0.06 | 0.03 | 0.06 | 0.05 | 0.11 | 0.04 |
| XTT | 0.66 | 0.81 | 0.85 | 0.91 | 0.90 | 1.00 | 1.05 | 1.11 | 1.20 | 0.23 | 0.11 | 0.04 | 0.05 | 0.03 | 0.05 | 0.02 | 0.03 | 0.01 |
| Nitro-TB | 0.33 | 0.41 | 0.41 | 0.42 | 0.44 | 0.39 | 0.42 | 0.45 | 0.43 | 0.09 | 0.10 | 0.09 | 0.07 | 0.06 | 0.02 | 0.01 | 0.02 | 0.01 |
| MTS | 0.36 | 0.42 | 0.41 | 0.43 | 0.43 | 0.48 | 0.59 | 0.63 | 0.66 | 0.07 | 0.06 | 0.06 | 0.07 | 0.03 | 0.03 | 0.02 | 0.00 | 0.04 |
| MTT | 0.34 | 0.36 | 0.35 | 0.35 | 0.33 | 0.35 | 0.37 | 0.41 | 0.52 | 0.15 | 0.08 | 0.08 | 0.07 | 0.01 | 0.02 | 0.04 | 0.01 | 0.06 |

TABLE 22
Formation of formazan dyes in the presence of control buffer, malate and 1-Methoxy PMS. The absorbance
was measured at the time indicated. Each mean and STDEV were derived from three experiments.

| Tetrazolium | Means | | | | | | | | | STDEV | | | | | | | | |
|---|---|---|---|---|---|---|---|---|---|---|---|---|---|---|---|---|---|---|
| dye | 0 h | 1 h | 2 h | 3 h | 4 h | 6 h | 8 h | 10 h | 12 h | 0 h | 1 h | 2 h | 3 h | 4 h | 6 h | 8 h | 10 h | 12 h |
| WST-1 | 0.31 | 0.31 | 0.32 | 0.32 | | | | | | 0.02 | 0.02 | 0.02 | 0.02 | | | | | |
| WST-4 | 0.13 | 0.13 | 0.13 | 0.15 | | | | | | 0.00 | 0.00 | 0.00 | 0.01 | | | | | |
| WST-5 | 0.13 | 0.15 | 0.16 | 0.16 | | | | | | 0.02 | 0.02 | 0.02 | 0.01 | | | | | |
| WST-8 | 0.19 | 0.20 | 0.23 | 0.26 | | | | | | 0.01 | 0.00 | 0.04 | 0.02 | | | | | |
| WST-9 | 0.18 | 0.20 | 0.19 | 0.23 | | | | | | 0.01 | 0.03 | 0.02 | 0.04 | | | | | |
| INT | 0.20 | 0.20 | 0.21 | 0.23 | 0.20 | 0.25 | 0.29 | 0.26 | 0.36 | 0.01 | 0.02 | 0.02 | 0.04 | 0.02 | 0.08 | 0.13 | 0.11 | 0.14 |
| XTT | 0.42 | 0.48 | 0.53 | 0.59 | 0.60 | 0.77 | 0.91 | 1.03 | 1.17 | 0.03 | 0.03 | 0.02 | 0.04 | 0.01 | 0.04 | 0.03 | 0.03 | 0.08 |
| Nitro-TB | 0.16 | 0.16 | 0.17 | 0.19 | 0.19 | 0.24 | 0.29 | 0.18 | 0.23 | 0.01 | 0.00 | 0.01 | 0.02 | 0.03 | 0.03 | 0.02 | 0.06 | 0.05 |
| MTS | 0.20 | 0.20 | 0.21 | 0.23 | 0.25 | 0.33 | 0.36 | 0.47 | 0.50 | 0.01 | 0.01 | 0.01 | 0.01 | 0.01 | 0.00 | 0.05 | 0.10 | 0.11 |
| MTT | 0.13 | 0.14 | 0.15 | 0.15 | 0.17 | 0.18 | 0.25 | 0.23 | 0.22 | 0.00 | 0.01 | 0.01 | 0.01 | 0.00 | 0.02 | 0.01 | 0.02 | 0.07 |

### Example 6

### Concentrations of Tetrazolium Salts, NAD+, Malate and Malate Dehydrogenase on the Rate of Formazan Formation

On the idea that higher concentrations of tetrazolium salts in the assay buffer would likely result in more formazan dyes in the reaction, various concentrations of tetrazolium salts were added to a reaction buffer and the formation of formazan dyes were measured at 0, 30 and 60 minutes. The reaction buffer was comprised of 1 mM MgCl2, 15 mM NADH+H, and 20 μg diaphorase in potassium phosphate 100 mM, pH 7.5. The reactions were performed at room temperature. Absorbance was taken at 0, 30 and 60 minutes.

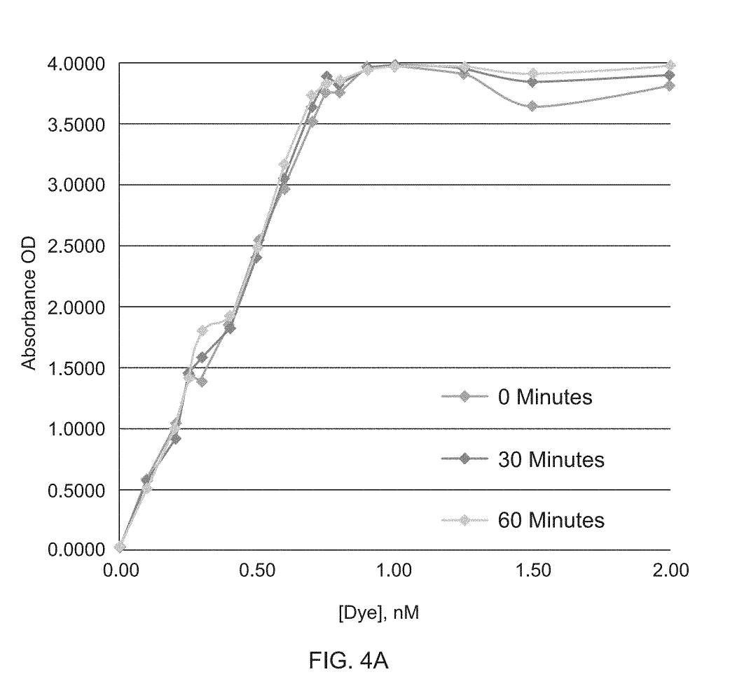

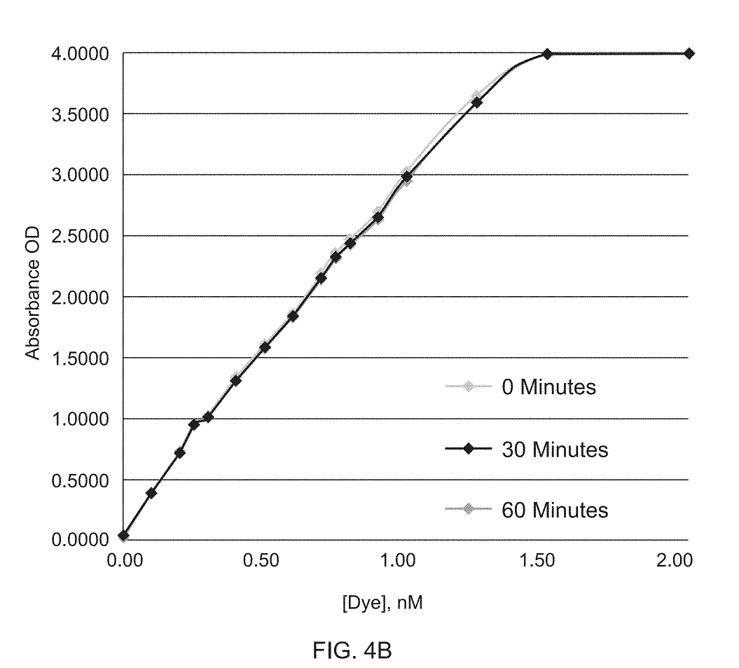

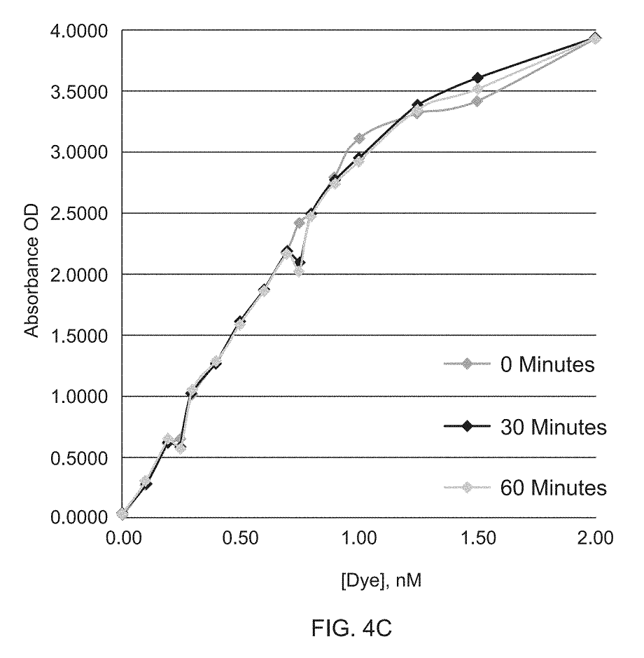

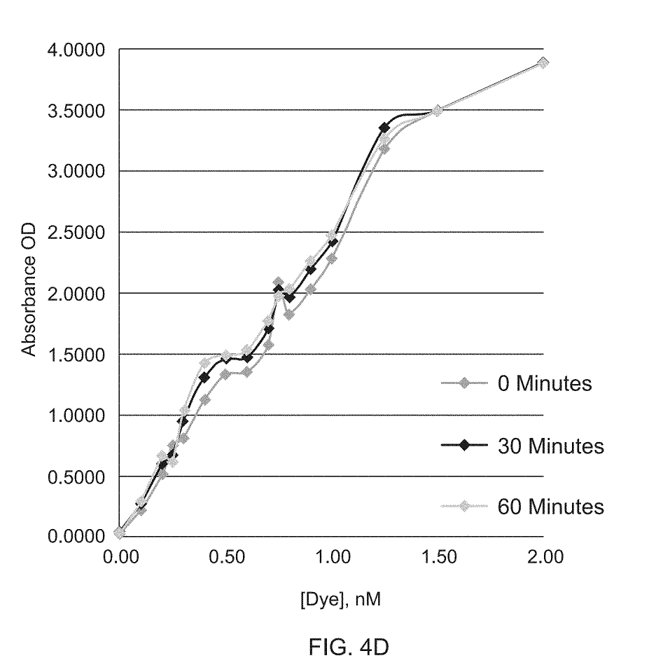

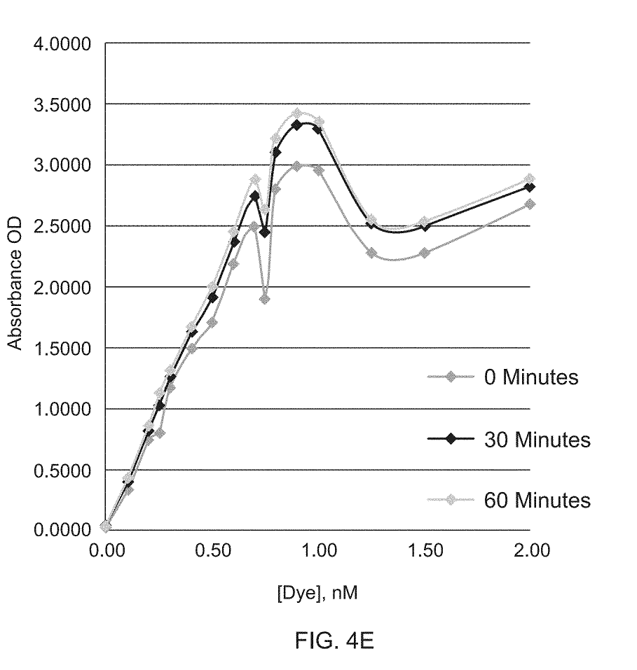

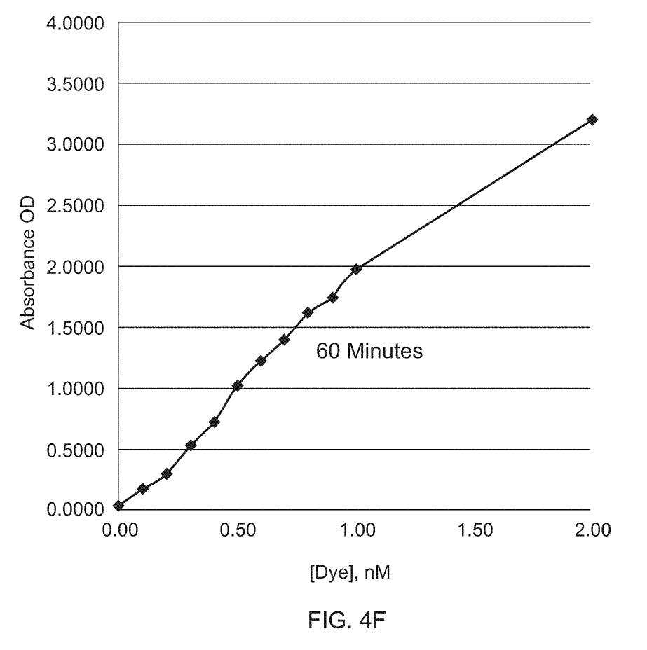

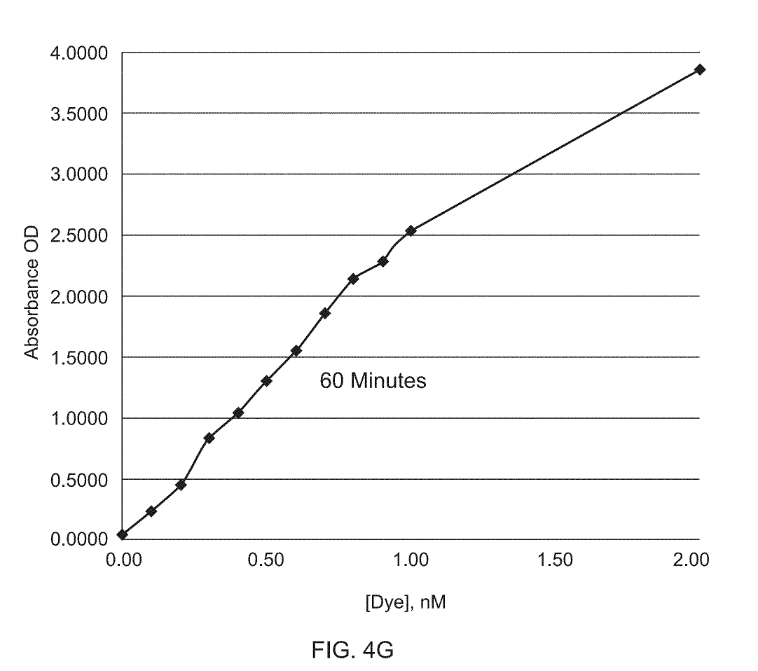

As shown in FIGS. 4A to 4G, the absorbance was highly correlated with the concentrations of tetrazolium salts in the reaction buffer. WST-5 reached peaks at 1 mM, while WST-8, EZMTT, MTT, INT did not reach peaks until 2 mM was added to the reaction buffer. WST-9 did not reach peaks even at 2 mM. The formazan salts of WST-9 started to form precipitates at 1 mM. MTS reached peaks around 1.5 mM.

To determine optimal conditions for quantifying enzymes in the gingival brush samples, oral lavage and gingival plaques, an experiment was carried out to determine the effect of NAD+ on conversion of tetrazolium salts to formazan dyes. A range of NAD+ concentrations from 100, 33.3, 11.1, 3.7, 1.2, 0.41, 0.13, 0.045, 0.015, 0.0051, 0.0017 and 0 was added to an assay medium containing: 4 μM rotenone, 1 mM MgCl2, 15 mM malate, 1.5 units of malate dehydrogenase, 2 mM WST-8 and 20 μg diaphorase in 100 mM potassium phosphate at pH 7.5. Absorbance was taken at every 5 minutes for 2 hours.

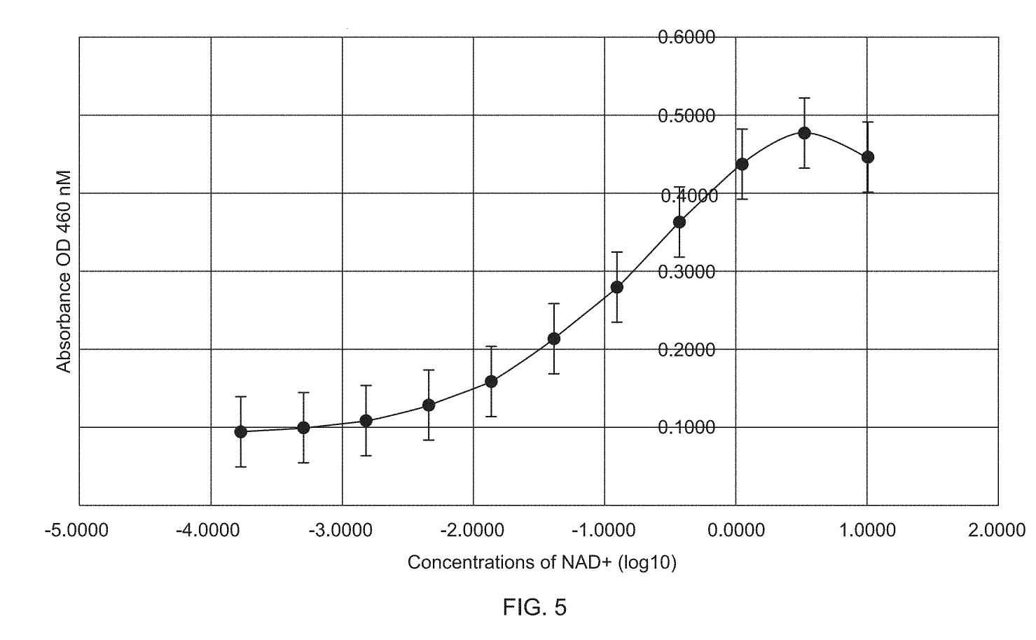

As shown in FIG. 5, the amount of formazan formation was proportional to the concentrations of NAD+ in the assay system. The higher NAD+ was in the assay buffer, the more formazan dyes were generated.

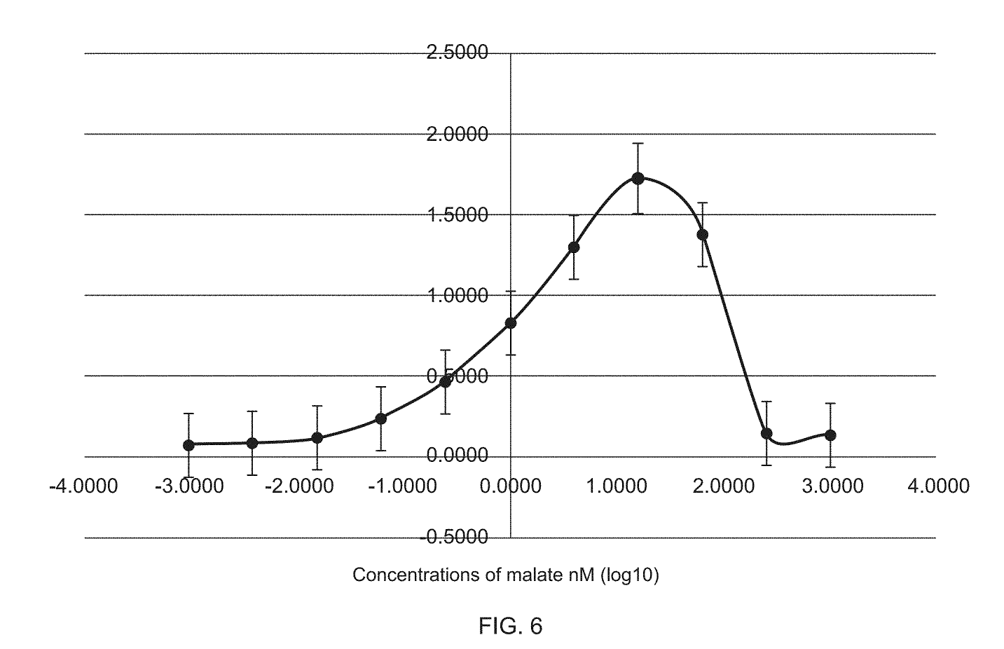

Substrate concentrations are important parameters in an enzymatic assay. An experiment was carried out to determine the effect of malate concentrations on formation of formazan dyes. Differing amounts of malate were added to an assay buffer, which comprised: 128.5 μM NAD+, 4 μM rotenone, 1 mM MgCl2, 1.5 units of malate dehydrogenase, 2 mM WST-8 and 20 μg diaphorase in 100 mM potassium phosphate at pH 7.5. Absorbance was measured every 5 minutes for 2 hours. As shown in FIG. 6, high concentrations of malate, (above 65 mM), inhibited production of formazan dyes. But at low concentrations from 0.001 mM to 15.6 mM, formation of formazan dyes was positively correlated with malate concentrations.

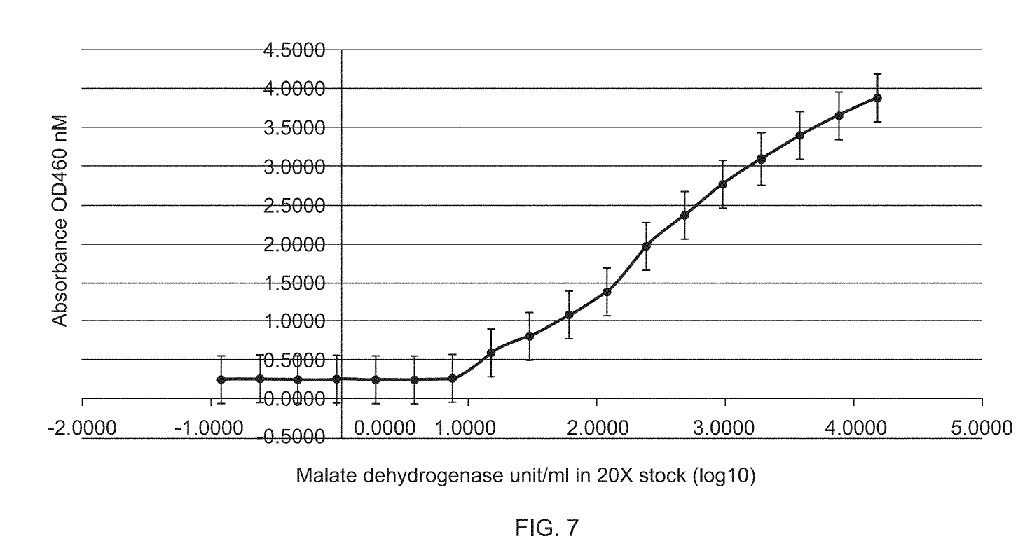

In oral lavage, gingival epithelium brush samples and gingival plaque samples, the amount of enzymes that metabolize glycose, amino acids, and fatty acids changes; depending on the healthy status of the oral tissues. The activities of the enzymes are indicative of oral tissue health status. Examples of indicative enzymes include: malate dehydrogenases, hexokinase, phosphohexose isomerase, phosphofructokinase, aldolase, triosephosphate isomerase, glyceraldehyde phosphate dehydrogenase, phosphoglycerate kinase, phosphoglycerate mutase, enolase, pyruvate kinase, lactate dehydrogenase, alcohol dehydrogenase, malate dehydrogenase, and other enzymes that participate in the tricarboxylic acid cycle or fatty acid and amino acid metabolism. Here, malate dehydrogenase was used to optimize an assay condition for formazan dye formation. Various amounts of malate dehydrogenase were added to an assay buffer which comprised 128.5 uM NAD+, 4 μM rotenone, 1 mM MgCl2, 15 mM malate, 2 mM WST-8 and 20 μg diaphorase in 100 mM potassium phosphate pH 7.5. Absorbance was measured every 5 minutes for 2 hours. As shown in FIG. 7, the absorbance at OD460 nM increased as more malate dehydrogenase was added to the reaction mix. This result showed that the assay buffer was able to quantitate malate dehydrogenase in the samples in the range of 15 to 15,000 units/ml.

### Example 7

### Profiling Proteins and Peptides in the Gingival Samples

A randomized, parallel group clinical study was conducted with 69 panelists (35 in the negative control group and 34 in the test regimen group). Panelists were 39 years old on average, ranging from 20 to 69, and 46% of the panelists were female. Treatment groups were well balanced, since there were no statistically significant (p≥0.395) differences for demographic characteristics (age, ethnicity, gender) or starting measurements for Gingival Bleeding Index (GBI); mean=29.957 with at least 20 bleeding sites, and Modified Gingival Index (MGI); mean=2.086. All sixty-nine panelists attended each visit and completed the research. The following treatment groups were compared over a 6-week period: Test regimen: Crest® Pro-Health Clinical Plaque Control (0.454% stannous fluoride) dentifrice; Oral-B® Professional Care 1000 with Precision Clean brush head and Crest® Pro-Health Refreshing Clean Mint (0.07% CPC) mouth rinse; Control regimen: Crest® Cavity Protection (0.243% sodium fluoride) dentifrice and Oral-B® Indicator Soft Manual toothbrush.

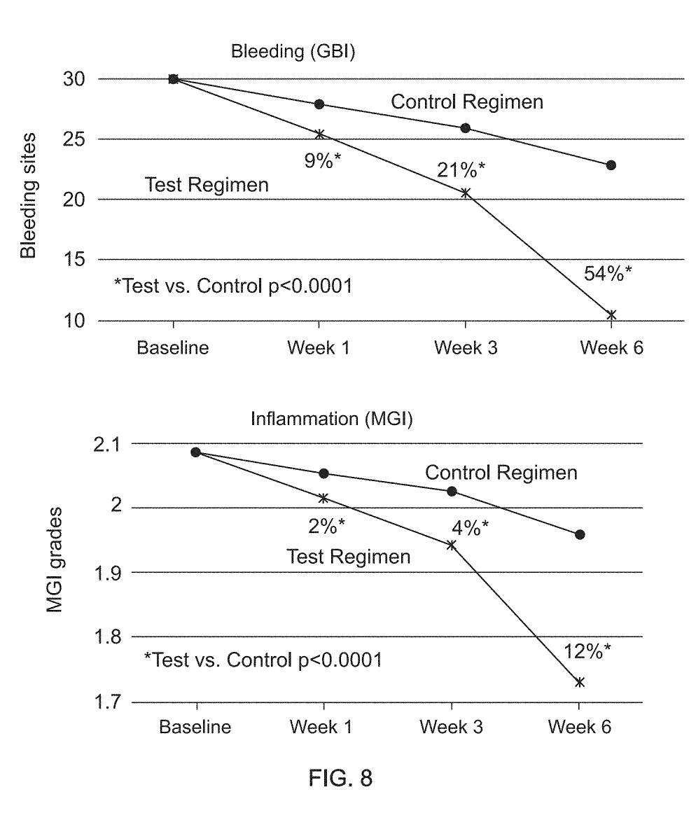

The test regimen group demonstrated significantly (p<0.0001) lower mean bleeding (GBI) and inflammation (MGI) relative to the negative control group at Weeks 1, 3 and 6, as shown in FIG. 8.

Gingival brush samples: Before sampling, panelists rinsed their mouths for 30 seconds with water. A dental hygienist then sampled the area just above the gumline using a buccal swab brush (Epicentre Biotechnologies, Madison, Wis.; cat. #MB100SP). At each sample site a brush was swabbed back-forth 10 times with the brush-head oriented parallel to the gum line. Each brush head was clipped off with sterile scissors and placed into a 15 ml conical tube with 800 ul DPBS (Dulbecco's phosphate-buffered saline), from Lifetechnologies, Grand Island, N.Y., containing 1× Halt™ Protease Inhibitor Single-Use Cocktail (Lifetechnologies). All gingival swabs from a given panelist were pooled into the same collection tube. All collection tubes were vigorously shaken on a multi-tube vortexer for 30 seconds at 4° C. Using sterile tweezers the brush heads were dabbed to the side of the tube to collect as much lysate as possible and subsequently discarded. Samples were immediately frozen on dry ice and stored in a −80° C. freezer until analysis. For analysis the samples were removed from the freezer, thawed and extracted by placing the samples on a tube shaker for 30 minutes at 4° C.; and then the tubes were centrifuged at 15000 RPM for 10 min in Eppendorf Centrifuge 5417R (Eppendorf, Ontario, Canada) to pellet any debris. The extract (800 μL) was analyzed for protein concentrations using the Bio-Rad protein assay (BioRad, Hercules, Calif.).

To reduce the sample numbers for proteomic study, protein samples from different panelists were pooled at baseline and week 3. Six pools were generated at baseline for the control and test regimens, respectively. Similarly, six pools were also generated for the control and test regimens at week 3, respectively. One baseline sample from the control regimen was excluded from analysis due to irregular output. Protein and peptide profiling were performed at the Yale W. M. Keck Foundation Biotechnology Resource Laboratory as described (Shibata S, Zhang J, Puthumana J, Stone K L, Lifton R P. Kelch-like 3 and Cullin 3 regulate electrolyte homeostasis via ubiquitination and degradation of WNK4. Proc Natl Ac ad Sci USA. 2013 May 7; 110(19):7838-43. doi: 10.1073/pnas.1304592110. Epub 2013 Apr. 1). Briefly, Proteins were digested with trypsin (modified sequencing grade, Sigma, St. Louis Mo.) overnight. Trypsin activity was quenched by acidification with trifluoroacetic acid, and peptide mixtures were fractionated by HPLC interfacing an electrospray ionisation quadrupole time-of-flight mass spectrometer. All MS/MS spectra were searched using the Mascot algorithm. Mascot is a powerful search engine used to identify proteins from LC-MS/MS data. See Matrix Science—Home (http://www.matrixscience.com/) for more details on this analysis.

Two hundred and eighty two peptides were found to be significantly different between the control and treatment regimens (P>0.05) or between baseline and week 3 (P<0.01) in either the control or treatment regimen. Those peptides represent 140 proteins (Each protein was cut into multiple peptides. In some instance, several peptides were derived from the same proteins.). TABLE 23 lists 140 proteins and peptides. Some of those peptides were derived from the following proteins: 14-3-3 protein epsilon, 14-3-3 protein sigma, Alpha-2-macroglobulin-like protein 1, Long-chain-fatty-acid-CoA ligase ACSBG1, Fructose-bisphosphate aldolase A, Alpha-amylase 1, Annexin A1, Calmodulin, Macrophage-capping protein, Cathepsin G, Carbonyl reductase [NADPH] 1, CD59 glycoprotein, 10 kDa heat shock protein, mitochondrial, Charged multivesicular body protein 4b, Clathrin light chain B, Complement C3, Cytochrome c, Cystatin-A, Cystatin-B, Desmoplakin, Destrin, Desmocollin-2, Extracellular matrix protein 1, Proteasome-associated protein ECM29 homolog, Elongation factor 1-alpha 1, Alpha-enolase, ERO1-like protein alpha, Ezrin, Protein FAM25A, Glucose-6-phosphate isomerase, Gelsolin, Glutamine synthetase, GDP-mannose 4,6 dehydratase, 78 kDa glucose-regulated protein, Glutathione S-transferase P, Histone H1.0, Hemoglobin subunit alpha, Hemoglobin subunit beta, E3 ubiquitin-protein ligase HECTD3, Heat shock protein beta-1, Calpastatin, Interleukin-1 receptor antagonist protein, Leukocyte elastase inhibitor, Involucrin, Creatine kinase U-type, mitochondrial, Laminin subunit gamma-1, L-lactate dehydrogenase A chain, Serine/threonine-protein kinase LMTK3, Malate dehydrogenase, mitochondrial, E3 ubiquitin-protein ligase MYCBP2, Neurofilament heavy polypeptide, Polyadenylate-binding protein 1, Protein disulfide-isomerase, Myeloperoxidase, Phosphoglycerate mutase 2, Phosphoglycerate kinase 1, Plectin, Peptidyl-prolyl cis-trans isomerase A, Peptidyl-prolyl cis-trans isomerase B, Peroxiredoxin-1, Peroxiredoxin-6, Pregnancy-specific beta-1-glycoprotein 8, Proteasome activator complex subunit 1, Cellular retinoic acid-binding protein 2, Protein S100-A8, Protein S100-A11, Protein S100-A16, Specifically androgen-regulated gene protein, Suprabasin, Protein SETSIP, Serpin B13, Serpin B3, Serpin B5, Small proline-rich protein 3, Small proline-rich protein 3, Translationally-controlled tumor protein, Transitional endoplasmic reticulum ATPase, Protein-glutamine gamma-glutamyltransferase E, Triosephosphate isomerase, Lactotransferrin, Uncharacterized protein DKFZp434B061, and Probable ribonuclease ZC3H12B.

TABLE 23
Peptides identified in the gingival brush samples.

| | | | | | | | P-value T-Test | | | |
|---|---|---|---|---|---|---|---|---|---|---|
| | | | | | | | Trt | Cntl | Trt | Trt |
| | | | | | | | Wk3 | Wk3 | Bsl | Wk3 |
| | | | Mean | | | | vs | vs | vs | vs |
| Uni | | | TrtB | TrtW | Cntl | Cntl | Trt | Cntl | Cnt | Cntl |
| Prot | Description | Sequence | sl | k3 | Bsl | Wk3 | Bsl | Bsl | 1Bsl | Wk3 |
| CH10_ | 10 kDa heat | DGDILGK | 623.0 | 1546.4 | 336.8 | 1115.3 | 0.021 | 0.101 | 0.459 | 0.187 |
| HUMAN | shock protein, | | | | | | | | | |
| | mitochondrial | | | | | | | | | |
| 1433B_ | 14-3-3 protein | AVTEQGHEL | 6474.2 | 10149.8 | 6129.8 | 9357.7 | 0.013 | 0.025 | 0.742 | 0.374 |
| HUMAN | beta/alpha | SNEER | | | | | | | | |
| 1433E_ | 14-3-3 protein | YLAEFATGN | 2804.5 | 5713.5 | 3802.4 | 6194.2 | 0.017 | 0.026 | 0.240 | 0.531 |
| HUMAN | epsilon | DRK | | | | | | | | |
| 1433S_ | 14-3-3 protein | EMPPTNPIR | 3283.7 | 6593.3 | 2008.9 | 4952.8 | 0.040 | 0.002 | 0.049 | 0.201 |
| HUMAN | sigma | | | | | | | | | |
| 1433Z_ | 14-3-3 protein | SVTEQGAEL | 186750.4 | 326090.7 | 190255.8 | 292907.6 | 0.034 | 0.024 | 0.891 | 0.518 |
| HUMAN | zeta/delta | SNEER | | | | | | | | |
| RS27_ | 40S ribosomal | DLLHPSPEE | 33752.2 | 45837.1 | 36248.5 | 47593.6 | 0.011 | 0.048 | 0.440 | 0.673 |
| HUMAN | protein S27 | EK | | | | | | | | |
| AL9A1_ | 4- | VEPADASGT | 5984.7 | 8802.5 | 4689.8 | 8624.4 | 0.029 | 0.064 | 0.184 | 0.915 |
| HUMAN | trimethylamino | EK | | | | | | | | |
| | butyraldehyde | | | | | | | | | |
| | dehydrogenase | | | | | | | | | |
| CH60_ | 60 kDa heat | VGGTSDVEV | 11806.1 | 18455.9 | 10415.4 | 17103.0 | 0.039 | 0.071 | 0.642 | 0.564 |
| HUMAN | shock protein, | NEK | | | | | | | | |
| | mitochondrial | | | | | | | | | |
| 6PGD_ | 6- | AGQAVDDFI | 10096.9 | 20075.9 | 12758.4 | 18840.3 | 0.036 | 0.029 | 0.070 | 0.744 |
| HUMAN | phospho- | EK | | | | | | | | |
| | gluconate | | | | | | | | | |
| | dehydrogenase, | | | | | | | | | |
| | decarboxyl- | | | | | | | | | |
| | ating | | | | | | | | | |
| GRP78_ | 78 kDa | VLEDSDLK | 9192.3 | 15225.7 | 7182.7 | 12846.8 | 0.001 | 0.043 | 0.335 | 0.069 |
| HUMAN | glucose- | | | | | | | | | |
| | regulated | | | | | | | | | |
| | protein | | | | | | | | | |
| ACTB_ | Actin, | EITALAPST | 41673.6 | 76926.2 | 36319.2 | 73810.4 | 0.030 | 0.008 | 0.438 | 0.799 |
| HUMAN | cytoplasmic 1 | MK | | | | | | | | |
| ARPC4_ | Actin-related | AENFFILR | 486.5 | 928.4 | 364.6 | 565.2 | 0.032 | 0.467 | 0.654 | 0.051 |
| HUMAN | protein 2/3 | | | | | | | | | |
| | complex | | | | | | | | | |
| | subunit 4 | | | | | | | | | |
| TCP4_ | Activated RNA | EQISDIDDA | 7363.3 | 11531.9 | 9699.4 | 9831.8 | 0.030 | 0.822 | 0.014 | 0.250 |
| HUMAN | polymerase II | VR | | | | | | | | |
| | transcriptional | | | | | | | | | |
| | coactivator p15 | | | | | | | | | |
| ADSV_ | Adseverin | TAEEFLQQM | 2147.3 | 5044.2 | 3094.1 | 4306.6 | 0.003 | 0.459 | 0.443 | 0.527 |
| HUMAN | | NYSK | | | | | | | | |
| FETUA_ | Alpha-2-HS- | HTLNQIDEV | 3584.3 | 5904.1 | 4239.5 | 5183.4 | 0.020 | 0.579 | 0.596 | 0.592 |
| HUMAN | glycoprotein | K | | | | | | | | |
| A2ML1_ | Alpha-2- | TFNIQSVNR | 8971.9 | 1413 2.9 | 7579.1 | 13769.2 | 0.035 | 0.096 | 0.215 | 0.914 |
| HUMAN | macroglobulin- | | | | | | | | | |
| | like protein 1 | | | | | | | | | |
| ACTN1_ | Alpha-actinin-1 | RDQALTEEH | 2471.3 | 1263.6 | 1660.8 | 1371.4 | 0.002 | 0.271 | 0.010 | 0.663 |
| HUMAN | | AR | | | | | | | | |
| ACTN4_ | Alpha-actinin-4 | DHGGALGPE | 8847.1 | 14536.6 | 7938.4 | 13691.8 | 0.013 | 0.025 | 0.563 | 0.616 |
| HUMAN | | EFK | | | | | | | | |
| ENOA_ | Alpha-enolase | LNVTEQEK | 33697.9 | 71243.0 | 30252.6 | 45538.4 | 0.024 | 0.352 | 0.761 | 0.152 |
| HUMAN | | | | | | | | | | |
| ANXA1_ | Annexin A1 | TPAQFDADE | 573737.1 | 1103967.7 | 529165.7 | 960205.2 | 0.001 | 0.011 | 0.678 | 0.073 |
| HUMAN | | LR | | | | | | | | |
| ANXA2_ | Annexin A2 | DLYDAGVKR | 39545.4 | 50535.7 | 25612.4 | 43936.0 | 0.103 | 0.000 | 0.059 | 0.006 |
| HUMAN | | | | | | | | | | |
| AATC_ | Aspartate | LALGDDSPA | 1731.2 | 3165.4 | 1835.9 | 3381.2 | 0.001 | 0.062 | 0.862 | 0.470 |
| HUMAN | amino- | LK | | | | | | | | |
| | transferase, | | | | | | | | | |
| | cytoplasmic | | | | | | | | | |
| ATPB_ | ATP synthase | IMDPNIVGS | 1275.7 | 3129.0 | 3158.5 | 3701.7 | 0.047 | 0.543 | 0.067 | 0.474 |
| HUMAN | subunit beta, | EHYDVAR | | | | | | | | |
| | mitochondrial | | | | | | | | | |
| RECQ5_ | ATP-dependent | ELLADLER | 3823.0 | 5623.5 | 3312.2 | 5483.6 | 0.031 | 0.029 | 0.565 | 0.621 |
| HUMAN | DNA helicase | | | | | | | | | |
| CALM_ | Calmodulin | DGNGYISAA | 34876.0 | 61067.7 | 48803.7 | 53288.5 | 0.033 | 0.619 | 0.140 | 0.431 |
| HUMAN | | ELR | | | | | | | | |
| CALL3_ | Calmodulin- | DTDNEEEIR | 9733.9 | 15869.0 | 5891.9 | 12940.4 | 0.046 | 0.004 | 0.073 | 0.191 |
| HUMAN | like protein 3 | | | | | | | | | |
| ICAL_ | Calpastatin | KTEKEESTE | 7723.3 | 11067.6 | 7447.8 | 9228.5 | 0.019 | 0.117 | 0.808 | 0.053 |
| HUMAN | | VLK | | | | | | | | |
| CALR_ | Calreticulin | GLQTSQDAR | 11499.9 | 16791.2 | 8830.2 | 15188.8 | 0.030 | 0.006 | 0.117 | 0.336 |
| HUMAN | | | | | | | | | | |
| CAH1_ | Carbonic | VLDALQAIK | 8.7 | 910.7 | 3.4 | 192.1 | 0.031 | 0.303 | 0.605 | 0.087 |
| HUMAN | anhydrase 1 | | | | | | | | | |
| CAMP_ | Cathelicidin | AIDGINQR | 5923.5 | 4078.8 | 4362.4 | 3844.6 | 0.045 | 0.719 | 0.298 | 0.758 |
| HUMAN | antimicrobial | | | | | | | | | |
| | peptide | | | | | | | | | |
| CATG_ | Cathepsin G | IFGSYDPR | 21478.2 | 12710.1 | 20293.9 | 17693.8 | 0.000 | 0.336 | 0.517 | 0.050 |
| HUMAN | | | | | | | | | | |
| CHM4B_ | Charged | KIEQELTAA | 616.9 | 1454.2 | 325.9 | 1287.6 | 0.031 | 0.028 | 0.285 | 0.611 |
| HUMAN | multivesicular | K | | | | | | | | |
| | body protein 4b | | | | | | | | | |
| CLIC1_ | Chloride | NSNPALNDN | 7569.2 | 16353.7 | 9055.6 | 15004.7 | 0.036 | 0.022 | 0.592 | 0.541 |
| HUMAN | intracellular | LEK | | | | | | | | |
| | channel protein | | | | | | | | | |
| | 1 | | | | | | | | | |
| CLCB_ | Clathrin light | RLQELDAAS | 1495.8 | 3625.8 | 945.1 | 3203.2 | 0.050 | 0.112 | 0.411 | 0.744 |
| HUMAN | chain B | K | | | | | | | | |
| CO3_ | Complement | TGLQEVEVK | 6462.6 | 7987.3 | 7378.4 | 7432.8 | 0.016 | 0.922 | 0.047 | 0.379 |
| HUMAN | C3 | | | | | | | | | |
| CRNN_ | Cornulin | LDQGNLHTS | 295264.9 | 482761.2 | 309640.1 | 501133.9 | 0.014 | 0.161 | 0.841 | 0.862 |
| HUMAN | | VSSAQGQDA | | | | | | | | |
| | | AQSEEK | | | | | | | | |
| COR1A_ | Coronin-1A | AAPEASGTP | 2381 5.5 | 16177.7 | 15288.9 | 19325.5 | 0.044 | 0.465 | 0.115 | 0.447 |
| HUMAN | | SSDAVSR | | | | | | | | |
| KCRU_ | Creatine kinase | ILENLR | 4549.5 | 7364.6 | 5907.6 | 6882.9 | 0.022 | 0.016 | 0.001 | 0.576 |
| HUMAN | U-type, | | | | | | | | | |
| | mitochondrial | | | | | | | | | |
| CYTA_ | Cystatin-A | VKPQLEEK | 14851.3 | 20781.0 | 8213.5 | 14863.4 | 0.061 | 0.057 | 0.094 | 0.018 |
| HUMAN | | | | | | | | | | |
| CYTB_ | Cystatin-B | AKHDELTYF | 350500.9 | 686593.2 | 402767.3 | 632435.7 | 0.008 | 0.022 | 0.351 | 0.534 |
| HUMAN | | | | | | | | | | |
| CYC_ | Cytochrome c | KTGQAPGYS | 3784.4 | 7949.3 | 4147.6 | 6023.8 | 0.014 | 0.048 | 0.641 | 0.116 |
| HUMAN | | YTAANK | | | | | | | | |
| DMKN_ | Dermokine | VGEAAHALG | 3281.4 | 7486.9 | 7330.6 | 7536.0 | 0.028 | 0.879 | 0.073 | 0.936 |
| HUMAN | | NTGHEIGR | | | | | | | | |
| DSC2_ | Desmocollin-2 | NLFYVER | 9081.8 | 15742.8 | 11058.5 | 17241.0 | 0.007 | 0.084 | 0.305 | 0.578 |
| HUMAN | | | | | | | | | | |
| DESP_ | Desmoplakin | GIVDSITGQ | 5624.2 | 10059.4 | 7712.9 | 7628.8 | 0.013 | 0.947 | 0.055 | 0.151 |
| HUMAN | | R | | | | | | | | |
| ODO2_ | Dihydrolipoyl- | TPAFAESVT | 20853.2 | 45660.4 | 30536.6 | 37348.6 | 0.041 | 0.561 | 0.261 | 0.508 |
| HUMAN | lysine-residue | EGDVR | | | | | | | | |
| | succinyltrans- | | | | | | | | | |
| | ferase compo- | | | | | | | | | |
| | nent of 2- | | | | | | | | | |
| | oxoglutarate | | | | | | | | | |
| | dehydrogenase | | | | | | | | | |
| | complex, | | | | | | | | | |
| | mitochondrial | | | | | | | | | |
| DNJB1_ | DnaJ homolog | GKDYYQTLG | 588.0 | 1472.7 | 1670.3 | 1765.4 | 0.018 | 0.821 | 0.038 | 0.360 |
| HUMAN | subfamily B | LAR | | | | | | | | |
| | member 1 | | | | | | | | | |
| MYCB2_ | E3 ubiquitin- | ACARELDGQ | 8051.1 | 63943.8 | 9187.8 | 22635.4 | 0.038 | 0.290 | 0.730 | 0.123 |
| HUMAN | protein ligase | EARQR | | | | | | | | |
| | MYCBP2 | | | | | | | | | |
| EF1A1_ | Elongation | LPLQDVYK | 873.0 | 2785.9 | 946.2 | 2257.5 | 0.004 | 0.020 | 0.741 | 0.286 |
| HUMAN | factor 1-alpha | | | | | | | | | |
| | 1 | | | | | | | | | |
| ERO1A_ | ERO1-like | LGAVDESLS | 50809.3 | 89111.5 | 58705.8 | 87660.3 | 0.011 | 0.041 | 0.452 | 0.877 |
| HUMAN | protein alpha | EETQK | | | | | | | | |
| ECM1_ | Extracellular | LLPAQLPAE | 11406.6 | 24555.1 | 13172.4 | 24637.4 | 0.029 | 0.118 | 0.771 | 0.985 |
| HUMAN | matrix protein | K | | | | | | | | |
| | 1 | | | | | | | | | |
| EZR1_ | Ezrin | EAQDDLVK | 1812 4.8 | 29354.9 | 13381.9 | 23893.0 | 0.008 | 0.046 | 0.283 | 0.055 |
| HUMAN | | | | | | | | | | |
| CAZA1_ | F-actin-capping | EASDPQPEE | 25245.0 | 32347.1 | 21583.3 | 27205.9 | 0.038 | 0.043 | 0.174 | 0.066 |
| HUMAN | protein subunit | ADGGLK | | | | | | | | |
| | alpha-1 | | | | | | | | | |
| FILA_ | Filaggrin | HSASQDGQD | 2441.8 | 903.3 | 1241.5 | 1838.4 | 0.015 | 0.296 | 0.051 | 0.104 |
| HUMAN | | TIR | | | | | | | | |
| ALDOA_ | Fructose- | RLQSIGTEN | 46120.0 | 95453.7 | 48319.3 | 73986.7 | 0.001 | 0.052 | 0.792 | 0.039 |
| HUMAN | bisphosphate | TEENRR | | | | | | | | |
| | aldolase A | | | | | | | | | |
| GMDS_ | GDP-mannose | VAFDELVR | 669.6 | 3459.8 | 336.8 | 1052.3 | 0.062 | 0.202 | 0.437 | 0.098 |
| HUMAN | 4,6 dehydratase | | | | | | | | | |
| GELS_ | Gelsolin | DSQEEEKTE | 7784.5 | 16910.5 | 5175.6 | 12191.3 | 0.001 | 0.075 | 0.115 | 0.169 |
| HUMAN | | ALTSAK | | | | | | | | |
| GLU2B_ | Glucosidase 2 | TVKEEAEKP | 828.0 | 1472.3 | 850.1 | 1573.2 | 0.007 | 0.044 | 0.934 | 0.455 |
| HUMAN | subunit beta | ER | | | | | | | | |
| GLNA_ | Glutamine | YIEEAIEK | 13589.1 | 26133.8 | 13608.7 | 20891.9 | 0.001 | 0.006 | 0.986 | 0.037 |
| HUMAN | synthetase | | | | | | | | | |
| GSTP1_ | Glutathione S- | TLGLYGK | 4071.6 | 9416.8 | 2925.0 | 7563.2 | 0.031 | 0.014 | 0.234 | 0.359 |
| HUMAN | transferase P | | | | | | | | | |
| GOGB1_ | Golgin | AQLKEIEAE | 13618.7 | 16567.4 | 10960.0 | 14976.9 | 0.048 | 0.307 | 0.205 | 0.638 |
| HUMAN | subfamily B | K | | | | | | | | |
| | member 1 | | | | | | | | | |
| HSP71_ | Heat shock 70 | YKAEDEVQR | 30544.9 | 38078.3 | 21846.5 | 33255.0 | 0.052 | 0.001 | 0.035 | 0.012 |
| HUMAN | kDa protein | | | | | | | | | |
| | 1A/1B | | | | | | | | | |
| HSPB1_ | Heat shock | AQLGGPEAA | 82641.1 | 47943.4 | 86986.6 | 56903.3 | 0.018 | 0.445 | 0.910 | 0.211 |
| HUMAN | protein beta-1 | KSDETAAK | | | | | | | | |
| HBA_ | Hemoglobin | VLSPADKTN | 44975.0 | 340255.2 | 36452.9 | 95049.1 | 0.009 | 0.375 | 0.646 | 0.042 |
| HUMAN | subunit alpha | VK | | | | | | | | |
| HBB_ | Hemoglobin | VNVDEVGGE | 79546.8 | 1001395.5 | 80565.4 | 252012.9 | 0.050 | 0.375 | 0.983 | 0.114 |
| HUMAN | subunit beta | ALGR | | | | | | | | |
| HMGB2_ | High mobility | IKSEHPGLS | 19129.2 | 10085.9 | 13849.4 | 11639.0 | 0.003 | 0.631 | 0.272 | 0.411 |
| HUMAN | group protein | IGDTAK | | | | | | | | |
| | B2 | | | | | | | | | |
| H10_ | Histone H1.0 | RLVTTGVLK | 4264.7 | 8798.0 | 2520.3 | 7680.7 | 0.044 | 0.034 | 0.396 | 0.448 |
| HUMAN | | | | | | | | | | |
| H15_ | Histone H1.5 | ALAAGGYDV | 27518.3 | 42195.0 | 30656.1 | 31134.3 | 0.000 | 0.897 | 0.334 | 0.009 |
| HUMAN | | EK | | | | | | | | |
| MYSM1_ | Histone H2A | DAVEAYQLA | 68195.5 | 17889.7 | 46262.7 | 20231.4 | 0.018 | 0.263 | 0.388 | 0.770 |
| HUMAN | deubiquitinase | QR | | | | | | | | |
| | MYSM1 | | | | | | | | | |
| H2B2F_ | Histone H2B | EIQTAVR | 24063.4 | 3033 | 21942.3 | 23502.2 | 0.092 | 0.838 | 0.788 | 0.040 |
| HUMAN | type 2-F | | | | | | | | | |
| INVO_ | Involucrin | HLVQQEGQL | 52897.6 | 80754.6 | 58457.2 | 7270 1.8 | 0.014 | 0.554 | 0.703 | 0.688 |
| HUMAN | | EQQER | | | | | | | | |
| IDHC_ | Isocitrate | TVEAEAAHG | 15482.9 | 17631.4 | 17771.7 | 13240.3 | 0.080 | 0.451 | 0.681 | 0.079 |
| HUMAN | dehydrogenase | TVTR | | | | | | | | |
| | [NADP] | | | | | | | | | |
| | cytoplasmic | | | | | | | | | |
| TRFL_ | Lactotrans- | LKQVLLHQQ | 15434.5 | 7532.6 | 13672.9 | 6956.6 | 0.009 | 0.063 | 0.573 | 0.648 |
| HUMAN | ferrin | AK | | | | | | | | |
| LAMC1_ | Laminin | LIEIASR | 15872.0 | 7213.4 | 16571.0 | 6151.9 | 0.039 | 0.069 | 0.881 | 0.704 |
| HUMAN | subunit | | | | | | | | | |
| | gamma-1 | | | | | | | | | |
| ILEU_ | Leukocyte | LGVQDLFNS | 9351.4 | 19761.2 | 8769.7 | 8901.6 | 0.069 | 0.973 | 0.915 | 0.007 |
| HUMAN | elastase | SK | | | | | | | | |
| | inhibitor | | | | | | | | | |
| LDHA_ | L-lactate | LNLVQR | 1210.4 | 2207.5 | 805.5 | 1964.9 | 0.015 | 0.024 | 0.223 | 0.459 |
| HUMAN | dehydrogenase | | | | | | | | | |
| | A chain | | | | | | | | | |
| LYSC_ | Lysozyme C | WESGYNTR | 286.6 | 783.6 | 685.6 | 419.4 | 0.009 | 0.436 | 0.207 | 0.126 |
| HUMAN | | | | | | | | | | |
| MIF_ | Macrophage | PMFIVNTNV | 5332.4 | 10109.7 | 8319.5 | 11969.6 | 0.040 | 0.140 | 0.201 | 0.319 |
| HUMAN | migration | PR | | | | | | | | |
| | inhibitory | | | | | | | | | |
| | factor | | | | | | | | | |
| CAPG_ | Macrophage- | EGNPEEDLT | 29622.1 | 46989.5 | 25960.8 | 42118.2 | 0.002 | 0.030 | 0.401 | 0.267 |
| HUMAN | capping protein | ADK | | | | | | | | |
| MDHM_ | Malate | ANTFVAELK | 1778.0 | 4639.0 | 2848.8 | 3731.2 | 0.006 | 0.307 | 0.190 | 0.222 |
| HUMAN | dehydrogenase, | | | | | | | | | |
| | mitochondrial | | | | | | | | | |
| MOES_ | Moesin | KAQQELEEQ | 3594.1 | 2149.5 | 2186.3 | 2082.8 | 0.026 | 0.804 | 0.054 | 0.789 |
| HUMAN | | TR | | | | | | | | |
| PERM_ | Myeloper- | RSPTLGASN | 28386.8 | 16648.3 | 25181.7 | 20462.4 | 0.016 | 0.345 | 0.386 | 0.410 |
| HUMAN | oxidase | R | | | | | | | | |
| MYH9_ | Myosin-9 | TDLLLEPYN | 875.8 | 1574.2 | 771.1 | 1533.5 | 0.039 | 0.112 | 0.781 | 0.885 |
| HUMAN | | K | | | | | | | | |
| NACAM_ | Nascent | IEDLSQQAQ | 2161.3 | 7139.8 | 6397.2 | 9293.3 | 0.003 | 0.172 | 0.005 | 0.282 |
| HUMAN | polypeptide- | LAAAEK | | | | | | | | |
| | associated | | | | | | | | | |
| | complex | | | | | | | | | |
| | subunit alpha, | | | | | | | | | |
| | muscle-specific | | | | | | | | | |
| | form | | | | | | | | | |
| NFH_ | Neurofilament | KLLEGEECR | 11145.3 | 2920.2 | 8545.7 | 5412.1 | 0.022 | 0.332 | 0.482 | 0.154 |
| HUMAN | heavy | | | | | | | | | |
| | polypeptide | | | | | | | | | |
| WIBG_ | Partner of Y14 | AAPTAASDQ | 3729.9 | 5686.7 | 3468.5 | 4789.6 | 0.024 | 0.437 | 0.787 | 0.544 |
| HUMAN | and mago | PDSAATTEK | | | | | | | | |
| PPIA_ | Peptidyl-prolyl | TAENFR | 10876.4 | 16838.5 | 10474.4 | 13854.3 | 0.022 | 0.066 | 0.773 | 0.147 |
| HUMAN | cis-trans | | | | | | | | | |
| | isomerase A | | | | | | | | | |
| PPIB_ | Peptidyl-prolyl | TVDNFVALA | 424.1 | 1663.6 | 1483.2 | 2313.8 | 0.040 | 0.283 | 0.113 | 0.332 |
| HUMAN | cis-trans | TGEK | | | | | | | | |
| | isomerase B | | | | | | | | | |
| PEPL_ | Periplakin | LSELEFHNS | 8446.6 | 5882.9 | 6810.3 | 6172.7 | 0.003 | 0.581 | 0.150 | 0.687 |
| HUMAN | | K | | | | | | | | |
| PRDX1_ | Peroxiredoxin- | TIAQDYGVL | 14423.1 | 30893.5 | 16697.8 | 26985.8 | 0.023 | 0.064 | 0.497 | 0.500 |
| HUMAN | 1 | K | | | | | | | | |
| PRDX2_ | Peroxiredoxin- | IGKPAPDFK | 4840.4 | 9887.5 | 2938.5 | 6338.7 | 0.006 | 0.043 | 0.126 | 0.036 |
| HUMAN | 2 | | | | | | | | | |
| PRDX6_ | Peroxiredoxin- | KLFPK | 1376.3 | 2985.7 | 1477.7 | 2558.2 | 0.005 | 0.083 | 0.832 | 0.259 |
| HUMAN | 6 | | | | | | | | | |
| PEX1_ | Peroxisome | GMMKELQTK | 1326.8 | 2861.3 | 2581.7 | 2565.0 | 0.003 | 0.983 | 0.163 | 0.323 |
| HUMAN | biogenesis | | | | | | | | | |
| | factor 1 | | | | | | | | | |
| PGKl_ | Phospho- | FHVEEEGKG | 5340.0 | 8397.1 | 4884.9 | 5952.9 | 0.043 | 0.428 | 0.703 | 0.101 |
| HUMAN | glycerate | K | | | | | | | | |
| | kinase 1 | | | | | | | | | |
| PLEC_ | Plectin | VPVDVAYR | 23162.7 | 46143.6 | 17501.7 | 40865.7 | 0.006 | 0.056 | 0.479 | 0.460 |
| HUMAN | | | | | | | | | | |
| PABP1_ | Polyadenylate- | KFEQMK | 56370.3 | 84565.0 | 42504.8 | 63429.8 | 0.039 | 0.054 | 0.281 | 0.012 |
| HUMAN | binding | | | | | | | | | |
| | protein 1 | | | | | | | | | |
| PSG8_ | Pregnancy- | SMTVKVSGK | 7117.7 | 13237.7 | 8855.0 | 11333.1 | 0.031 | 0.464 | 0.580 | 0.424 |
| HUMAN | specific | R | | | | | | | | |
| | beta-1- | | | | | | | | | |
| | glycoprotein | | | | | | | | | |
| | 8 | | | | | | | | | |
| GP146_ | Probable G- | LQRLMK | 7634.4 | 14633.5 | 9521.0 | 12181.9 | 0.001 | 0.036 | 0.005 | 0.105 |
| HUMAN | protein coupled | | | | | | | | | |
| | receptor 146 | | | | | | | | | |
| ZC12B_ | Probable | GVYARNPNL | 14702.2 | 1027.9 | 5903.5 | 1876.8 | 0.048 | 0.173 | 0.179 | 0.183 |
| HUMAN | ribonuclease | CSDSR | | | | | | | | |
| | ZC3H12B | | | | | | | | | |
| PROF1_ | Profilin-1 | EGVHGGLIN | 643.6 | 1039.7 | 653.0 | 731.5 | 0.037 | 0.779 | 0.969 | 0.168 |
| HUMAN | | K | | | | | | | | |
| PSME1_ | Proteasome | IENLLGSYF | 479.7 | 1982.2 | 1309.6 | 2136.1 | 0.042 | 0.037 | 0.056 | 0.766 |
| HUMAN | activator | PK | | | | | | | | |
| | complex | | | | | | | | | |
| | subunit 1 | | | | | | | | | |
| PSA3_ | Proteasome | AVENSSTAI | 2591.7 | 4133.7 | 4680.6 | 4309.5 | 0.024 | 0.807 | 0.164 | 0.845 |
| HUMAN | subunit alpha | GIR | | | | | | | | |
| | type-3 | | | | | | | | | |
| PSB6_ | Proteasome | TTTGSYIAN | 4184.4 | 5054.8 | 4537.9 | 4417.4 | 0.021 | 0.882 | 0.617 | 0.237 |
| HUMAN | subunit beta | R | | | | | | | | |
| | type-6 | | | | | | | | | |
| ECM29_ | Proteasome- | LSSTQEGVR | 83586.4 | 29450.7 | 68098.3 | 31331.7 | 0.002 | 0.164 | 0.521 | 0.788 |
| HUMAN | associated | K | | | | | | | | |
| | protein ECM29 | | | | | | | | | |
| | homolog | | | | | | | | | |
| PDIA1_ | Protein | YQLDK | 17143.1 | 27359.8 | 14218.1 | 23271.7 | 0.024 | 0.009 | 0.374 | 0.087 |
| HUMAN | disulfide- | | | | | | | | | |
| | isomerase | | | | | | | | | |
| FM25A_ | Protein | LAAEGLAHR | 1647.2 | 2857.6 | 1016.6 | 1517.2 | 0.047 | 0.350 | 0.214 | 0.047 |
| HUMAN | FAM25A | | | | | | | | | |
| PRC2B_ | Protein | QDQQDPK | 3876.0 | 6095.8 | 2539.4 | 3767.8 | 0.027 | 0.120 | 0.168 | 0.005 |
| HUMAN | PRRC2B | | | | | | | | | |
| S10AB_ | Protein S100- | NQKDPGVLD | 124467.2 | 218832.8 | 109449.5 | 169693.8 | 0.014 | 0.095 | 0.443 | 0.187 |
| HUMAN | A11 | R | | | | | | | | |
| S10AG_ | Protein S100- | LIHEQEQQS | 8702.5 | 15985.5 | 6388.6 | 10985.9 | 0.025 | 0.077 | 0.386 | 0.032 |
| HUMAN | A16 | SS | | | | | | | | |
| S10A6_ | Protein S100- | LQDAEIAR | 167980.2 | 219750.4 | 116779.4 | 176514.2 | 0.030 | 0.010 | 0.021 | 0.045 |
| HUMAN | A6 | | | | | | | | | |
| S10A8_ | Protein S100- | KLLETECPQ | 14185.0 | 31460.6 | 38968.7 | 41008.0 | 0.049 | 0.854 | 0.028 | 0.376 |
| HUMAN | A8 | YIRK | | | | | | | | |
| S10A9_ | Protein S100- | DLQNFLK | 411383.9 | 648948.4 | 483769.1 | 545150.1 | 0.003 | 0.109 | 0.036 | 0.070 |
| HUMAN | A9 | | | | | | | | | |
| SETLP_ | Protein SETSIP | RSELIAK | 809.2 | 1620.6 | 722.3 | 1133.6 | 0.043 | 0.140 | 0.754 | 0.116 |
| HUMAN | | | | | | | | | | |
| TGM3_ | Protein- | VPDESEVVV | 40923.6 | 70843.9 | 31482.5 | 51193.2 | 0.017 | 0.036 | 0.212 | 0.060 |
| HUMAN | glutamine | ER | | | | | | | | |
| | gamma- | | | | | | | | | |
| | glutamyl- | | | | | | | | | |
| | transferase E | | | | | | | | | |
| PTMA_ | Prothymosin | RAAEDDEDD | 3550.8 | 5186.4 | 3114.9 | 4841.1 | 0.046 | 0.047 | 0.504 | 0.589 |
| HUMAN | alpha | DVDTKK | | | | | | | | |
| KPYM_ | Pyruvate kinase | GSGTAEVEL | 16658.2 | 23529.4 | 12918.4 | 19760.9 | 0.011 | 0.004 | 0.039 | 0.065 |
| HUMAN | PKM | KK | | | | | | | | |
| GDIB_ | Rab GDP | TFEGIDPK | 4080.5 | 5987.0 | 3885.3 | 5618.5 | 0.043 | 0.012 | 0.762 | 0.472 |
| HUMAN | dissociation | | | | | | | | | |
| | inhibitor beta | | | | | | | | | |
| GDIR2_ | Rho GDP- | APNVVVTR | 5082.8 | 2536.9 | 3520.1 | 2670.1 | 0.021 | 0.312 | 0.174 | 0.714 |
| HUMAN | dissociation | | | | | | | | | |
| | inhibitor 2 | | | | | | | | | |
| RINI_ | Ribonuclease | ELTVSNNDI | 5705.2 | 13521.2 | 13042.9 | 15417.3 | 0.012 | 0.219 | 0.014 | 0.316 |
| HUMAN | inhibitor | NEAGVR | | | | | | | | |
| LMTK3_ | Serine/ | APGIEEK | 58040.2 | 104244.7 | 37038.8 | 76294.9 | 0.002 | 0.025 | 0.086 | 0.037 |
| HUMAN | threonine- | | | | | | | | | |
| | protein | | | | | | | | | |
| | kinase LMTK3 | | | | | | | | | |
| TRFE_ | Serotrans- | DSAHGFLK | 11823.8 | 16197.3 | 9903.5 | 11794.2 | 0.045 | 0.267 | 0.361 | 0.012 |
| HUMAN | ferrin | | | | | | | | | |
| SPB13_ | Serpin B13 | TYLFLQK | 528.9 | 1816.3 | 671.5 | 1401.6 | 0.045 | 0.009 | 0.360 | 0.412 |
| HUMAN | | | | | | | | | | |
| SPB3_ | Serpin B3 | VLHFDQVTE | 12335.0 | 31865.2 | 16544.0 | 25073.8 | 0.028 | 0.072 | 0.472 | 0.184 |
| HUMAN | | NTTGK | | | | | | | | |
| SPB5_ | Serpin B5 | DVEDESTGL | 18881.3 | 33124.8 | 14492.1 | 21720.3 | 0.009 | 0.129 | 0.325 | 0.016 |
| HUMAN | | EK | | | | | | | | |
| ALBU_ | Serum albumin | DDNPNLPR | 125593.7 | 150718.4 | 61878.9 | 102025.1 | 0.065 | 0.083 | 0.017 | 0.015 |
| HUMAN | | | | | | | | | | |
| SSBP_ | Single-stranded | SGDSEVYQL | 5558.2 | 10917.0 | 7882.5 | 10686.9 | 0.014 | 0.17 | 0.242 | 0.867 |
| HUMAN | DNA-binding | GDVSQK | | | | | | | | |
| | protein, | | | | | | | | | |
| | mitochondrial | | | | | | | | | |
| SPRR3_ | Small proline- | VPVPGYTK | 212243.1 | 394037.6 | 167604.6 | 348856.5 | 0.003 | 0.056 | 0.490 | 0.371 |
| HUMAN | rich protein 3 | | | | | | | | | |
| SARG_ | Specifically | | | | | | | | | |
| HUMAN | androgen- | AEDAPLSSG | 6421.1 | 10908.1 | 9103.6 | 12683.0 | 0.045 | 0.222 | 0.224 | 0.475 |
| | regulated gene | EDPNSR | | | | | | | | |
| | protein | | | | | | | | | |
| GRP75_ | Stress-70 | VLENAEGAR | 5750.4 | 8750.7 | 4681.5 | 6879.2 | 0.086 | 0.080 | 0.524 | 0.025 |
| HUMAN | protein, | | | | | | | | | |
| | mitochondrial | | | | | | | | | |
| SBSN_ | Suprabasin | FGQGAHHAA | 62030.8 | 82870.1 | 57228.2 | 65066.6 | 0.038 | 0.413 | 0.674 | 0.003 |
| HUMAN | | GQAGNEAGR | | | | | | | | |
| THIO_ | Thioredoxin | VGEFSGANK | 257150.7 | 394704.2 | 221041.9 | 341085.6 | 0.016 | 0.001 | 0.143 | 0.157 |
| HUMAN | | | | | | | | | | |
| TYPH_ | Thymidine | ALQEALVLS | 1282.9 | 3282.4 | 1774.9 | 3512.2 | 0.007 | 0.087 | 0.435 | 0.740 |
| HUMAN | phosphorylase | DR | | | | | | | | |
| TALDO_ | Transaldolase | SYEPLEDPG | 19378.8 | 28463.8 | 18752.3 | 25742.5 | 0.008 | 0.023 | 0.770 | 0.196 |
| HUMAN | | VK | | | | | | | | |
| TERA_ | Transitional | LAGESESNL | 6262.0 | 11337.6 | 5692.4 | 9616.4 | 0.035 | 0.015 | 0.441 | 0.385 |
| HUMAN | endoplasmic | RK | | | | | | | | |
| | reticulum | | | | | | | | | |
| | ATPase | | | | | | | | | |
| TCTP_ | Transla- | GKLEEQRPE | 1438.3 | 2941.9 | 1566.9 | 2422.8 | 0.051 | 0.001 | 0.521 | 0.378 |
| HUMAN | tionally- | R | | | | | | | | |
| | controlled | | | | | | | | | |
| | tumor protein | | | | | | | | | |
| TPIS_ | Triosephos- | VIADNVK | 4916.5 | 9475.5 | 4301.4 | 7296.5 | 0.001 | 0.014 | 0.367 | 0.025 |
| HUMAN | phate isomerase | | | | | | | | | |
| RS27A_ | Ubiquitin-40S | TLSDYNIQK | 16297.6 | 28274.7 | 13652.7 | 25475.3 | 0.023 | 0.028 | 0.564 | 0.313 |
| HUMAN | ribosomal | | | | | | | | | |
| | protein S27a | | | | | | | | | |
| UB2V1_ | Ubiquitin- | LLEELEEGQ | 2880.5 | 6110.0 | 3307.2 | 5580.5 | 0.003 | 0.032 | 0.317 | 0.530 |
| HUMAN | conjugating | K | | | | | | | | |
| | enzyme E2 | | | | | | | | | |
| | variant 1 | | | | | | | | | |
| RD23B_ | UV excision | TLQQQTFK | 2542.4 | 3945.9 | 954.6 | 2401.6 | 0.021 | 0.040 | 0.025 | 0.021 |
| HUMAN | repair protein | | | | | | | | | |
| | RAD23 | | | | | | | | | |
| | homolog B | | | | | | | | | |
| YBOX3_ | Y-box-binding | GAEAANVTG | 8580.6 | 15477.7 | 9414.4 | 13549.8 | 0.028 | 0.070 | 0.744 | 0.172 |
| HUMAN | protein 3 | PDGVPVEGS | | | | | | | | |
| | | R | | | | | | | | |
| ZN185_ | Zinc finger | RVEVVEEDG | 1304.0 | 2653.4 | 4594.2 | 6167.6 | 0.041 | 0.780 | 0.410 | 0.418 |
| HUMAN | protein 185 | PSEK | | | | | | | | |

### Example 8

### Multiple Substrates into One Assay

Malate dehydrogenase catalyzes the conversion of malate into oxaloacetate and reduces oxidized nicotinamide adenine dinucleotide (NAD) to reduced nicotinamide adenine dinucleotide (NADH). Similarly, glyceraldehyde-3-phosphate dehydrogenase catalyzes oxidative phosphorylation of glyceraldehyde-3-phosphate in the presence of inorganic phosphate and reduces NAD to NADH. NADH can reduce tetrazolium salts, such as WST-1, WST-5, WST-8, WST-9, MTT, MTS, Nitro-Blue, INT and EZMTT, into formazan pigments to generate distinctive colors. As described in Example 4, oral lavage samples from gingivitis panelists had higher activities of malate dehydrogenase and triosephosphate isomerase which can convert tetrazolium salts into formazan products. Mixtures of both malate dehydrogenase and triosephosphate substrates speed the conversion of tetrazolium salts into formazan products.

### Example 9

### Resazurin Reduction Activities in Oral Lavage

A clinical study was conducted, as described in Example 1, to evaluate sample collection methods and measurement procedures. It was a controlled, examiner-blind study. Forty panelists satisfying the inclusion/exclusion criteria were enrolled. Twenty (20) panelists were qualified as healthy—with up to 3 bleeding sites and with all pockets less than or equal to 2 mm deep and twenty (20) panelists were qualified as unhealthy—greater than 20 bleeding sites with at least 3 pockets greater than or equal to 3 mm but not deeper than 4 mm with bleeding, and at least 3 pockets less than or equal to 2 mm deep with no bleeding for sampling. All panelists were given investigational products: Crest® Pro-Health Clinical Gum Protection Toothpaste (0.454% stannous fluoride) and Oral-B® Indicator Soft Manual Toothbrush. Panelists continued their regular oral hygiene routine, and did not use any new products starting from the baseline to the end of four week treatment study. During the four week treatment period, panelists brushed their teeth twice daily, morning and evening, in their customary manner using the assigned dentifrice and soft manual toothbrush.

Oral lavage samples were collected at wake up (one per panelist) by rinsing with 4 ml of water for 30 seconds and then expectorating the contents of the mouth into a centrifuge tube. These samples were frozen at home until they were brought into a test site in a cold pack. Each panelist provided up to 15 samples throughout the study. Oral lavage samples at a test site were frozen at −70° C.

Oral lavage samples (150 μl) at baseline and week 4 treatment of 20 healthy panelists and 18 healthy panelists were sent to Metabolon (Morrisville, N.C. 27560) for metabolite profiling. All samples were analyzed using Metabolon's global biochemical profiling platforms. In brief, samples were extracted and split into equal parts for analysis on the LC (liquid chromatography)/MS (mass spectrometry)/MS and Polar LC platforms. Proprietary software was used to match ions to an in-house library of standards for metabolite identification and for metabolite quantitation by peak area integration.

As shown in TABLE 24, succinate, malate, fumarate, phosphoenolpyruvate (PEP) and lactate are presented in oral lavage samples. Succinate, malate, fumarate and lactate are substrates for succinate dehydrogenase, malate dehydrogenase and lactate dehydrogenase, respectively.

As shown in TABLE 1, malate dehydrogenase and lactate dehydrogenase are increased in the lavage of unhealthy panelists in comparison with those in the lavage samples of healthy panelists. Both malate dehydrogenase and lactate dehydrogenase can catalyze oxidation of their respective substrates, and reduce NAD to NADH at the same time. NADH in turn can reduce tetrazolium salts or resazurin into formazan dyes and resorufin, respectively, in the presence of diaphorase or other electron carriers.

TABLE 24
Metabolites in oral lavage samples

| | | Ratios | | | | Statistical p Values | | | |
|---|---|---|---|---|---|---|---|---|---|
| | | | | | Unhealthy | | | | Unhealthy |
| | | Healthy | Unhealthy | Unhealthy | Wk 4/ | Healthy | Unhealthy | Unhealthy | Wk 4/ |
| | | Wk 4/ | Wk 4/ | BL/ | Low | Wk 4/ | Wk 4/ | BL/ | Low |
| Biochemical | | Healthy | Unhealthy | Healthy | Healthy | Healthy | Unhealthy | Healthy | Healthy |
| Name | PUBCHEM | BL | BL | BL | Wk 4 | BL | BL | BL | Wk 4 |
| glycine | 750 | 1.24 | 1.33 | 1.11 | 1.19 | 0.11 | 0.05 | 0.69 | 0.50 |
| N-acetylglycine | 10972 | 1.06 | 1.04 | 1.11 | 1.09 | 0.61 | 0.75 | 0.63 | 0.69 |
| sarcosine (N- | 1088 | 1.15 | 1.02 | 1.17 | 1.03 | 0.26 | 0.89 | 0.53 | 0.89 |
| Methylglycine) | | | | | | | | | |
| dimethylglycine | 673 | 0.97 | 0.81 | 1.31 | 1.09 | 0.83 | 0.15 | 0.30 | 0.73 |
| betaine | 247 | 0.96 | 0.77 | 1.23 | 0.98 | 0.67 | 0.02 | 0.25 | 0.93 |
| serine | 5951 | 1.6 | 1.44 | 1.45 | 1.31 | 0.01 | 0.04 | 0.19 | 0.34 |
| N-acetylserine | 65249 | 0.95 | 0.85 | 1.49 | 1.33 | 0.62 | 0.16 | 0.08 | 0.21 |
| threonine | 6288 | 1.39 | 1.09 | 1.9 | 1.49 | 0.09 | 0.68 | 0.04 | 0.20 |
| N-acetylthreonine | 152204 | 0.91 | 0.77 | 1.53 | 1.31 | 0.54 | 0.14 | 0.12 | 0.32 |
| O- | 439389 | 0.74 | 0.57 | 1.04 | 0.8 | 0.07 | 0.00 | 0.91 | 0.50 |
| acetylhomoserine | | | | | | | | | |
| alanine | 5950 | 1.06 | 0.87 | 1.56 | 1.28 | 0.69 | 0.33 | 0.08 | 0.32 |
| N-acetylalanine | 88064 | 0.95 | 0.72 | 1.54 | 1.16 | 0.74 | 0.03 | 0.11 | 0.58 |
| aspartate | 5960 | 1.14 | 1.17 | 1.57 | 1.61 | 0.36 | 0.31 | 0.08 | 0.07 |
| asparagine | 6267 | 3.31 | 3.41 | 0.82 | 0.84 | 0.00 | 0.00 | 0.56 | 0.61 |
| N- | 99715 | 0.77 | 0.69 | 1.42 | 1.27 | 0.14 | 0.04 | 0.22 | 0.40 |
| acetylasparagine | | | | | | | | | |
| N-acetylaspartate | 65065 | 0.98 | 0.8 | 1.43 | 1.17 | 0.84 | 0.09 | 0.13 | 0.49 |
| (NAA) | | | | | | | | | |
| glutamate | 611 | 0.91 | 0.83 | 1.55 | 1.42 | 0.48 | 0.20 | 0.08 | 0.16 |
| glutamine | 5961 | 1.44 | 1.55 | 1.3 | 1.4 | 0.10 | 0.06 | 0.44 | 0.32 |
| N-acetylglutamate | 70914 | 0.8 | 0.64 | 1.48 | 1.17 | 0.13 | 0.00 | 0.12 | 0.52 |
| N-acetylglutamine | 182230 | 1.15 | 0.86 | 1.11 | 0.83 | 0.26 | 0.25 | 0.66 | 0.43 |
| gamma- | 119 | 0.64 | 0.53 | 1.29 | 1.08 | 0.00 | 0.00 | 0.29 | 0.76 |
| aminobutyrate | | | | | | | | | |
| (GABA) | | | | | | | | | |
| glutamate, | 68662 | 1.01 | 0.95 | 1.61 | 1.5 | 0.93 | 0.71 | 0.05 | 0.09 |
| gamma-methyl | | | | | | | | | |
| ester | | | | | | | | | |
| pyroglutamine* | 134508 | 0.97 | 0.83 | 1.96 | 1.69 | 0.79 | 0.19 | 0.02 | 0.06 |
| histidine | 6274 | 1.5 | 1.61 | 1.26 | 1.34 | 0.04 | 0.02 | 0.43 | 0.31 |
| N-acetylhistidine | 75619 | 0.81 | 0.64 | 1.43 | 1.13 | 0.25 | 0.03 | 0.29 | 0.71 |
| 1-methylhistidine | 92105 | 0.93 | 0.72 | 1.54 | 1.2 | 0.66 | 0.08 | 0.20 | 0.58 |
| 3-methylhistidine | 64969 | 0.59 | 0.76 | 1.33 | 1.71 | 0.05 | 0.32 | 0.43 | 0.14 |
| trans-urocanate | 736715 | 1.11 | 0.79 | 1.83 | 1.3 | 0.47 | 0.12 | 0.01 | 0.28 |
| cis-urocanate | 1549103 | 1.03 | 0.89 | 1.08 | 0.94 | 0.86 | 0.55 | 0.75 | 0.80 |
| formiminoglutamate | 439233 | 0.7 | 0.69 | 1.08 | 1.07 | 0.04 | 0.05 | 0.77 | 0.80 |
| imidazole | 70630 | 0.92 | 0.71 | 1.26 | 0.97 | 0.51 | 0.01 | 0.41 | 0.90 |
| propionate | | | | | | | | | |
| imidazole lactate | 440129 | 0.92 | 0.82 | 1.29 | 1.14 | 0.59 | 0.20 | 0.41 | 0.67 |
| histamine | 774 | 0.81 | 0.46 | 2.77 | 1.59 | 0.39 | 0.01 | 0.04 | 0.34 |
| 4-imidazoleacetate | 96215 | 1.07 | 0.73 | 1.53 | 1.04 | 0.64 | 0.05 | 0.12 | 0.89 |
| N-acetylhistamine | 69602 | 0.73 | 0.45 | 2.94 | 1.82 | 0.15 | 0.00 | 0.02 | 0.20 |
| lysine | 5962 | 1.21 | 1.16 | 1.39 | 1.33 | 0.19 | 0.33 | 0.22 | 0.28 |
| N2- | | 1.08 | 0.88 | 1.54 | 1.25 | 0.61 | 0.43 | 0.18 | 0.48 |
| acetyllysine/N6- | | | | | | | | | |
| acetyllysine | | | | | | | | | |
| N6,N6,N6- | 440120 | 0.97 | 0.72 | 1.71 | 1.26 | 0.88 | 0.09 | 0.16 | 0.53 |
| trimethyllysine | | | | | | | | | |
| 5-hydroxylysine | 1029 | 0.8 | 0.56 | 1.19 | 0.83 | 0.17 | 0.00 | 0.42 | 0.39 |
| saccharopine | 160556 | 1 | 0.78 | 1.72 | 1.34 | 1.00 | 0.18 | 0.05 | 0.28 |
| 2-aminoadipate | 469 | 0.88 | 0.76 | 1.55 | 1.33 | 0.28 | 0.03 | 0.04 | 0.17 |
| glutarate | 743 | 0.92 | 0.71 | 1.25 | 0.97 | 0.53 | 0.02 | 0.26 | 0.86 |
| (pentanedioate) | | | | | | | | | |
| pipecolate | 849 | 1 | 0.66 | 1.39 | 0.91 | 0.98 | 0.03 | 0.29 | 0.76 |
| cadaverine | 273 | 1.1 | 0.79 | 1.12 | 0.8 | 0.56 | 0.17 | 0.73 | 0.52 |
| 5-aminovalerate | 138 | 0.67 | 0.61 | 1.16 | 1.06 | 0.00 | 0.00 | 0.43 | 0.77 |
| phenylalanine | 6140 | 1.24 | 1.09 | 1.42 | 1.24 | 0.13 | 0.57 | 0.16 | 0.38 |
| N- | 74839 | 0.99 | 0.73 | 1.14 | 0.84 | 0.93 | 0.06 | 0.61 | 0.51 |
| acetylphenylalanine | | | | | | | | | |
| phenylpyruvate | 997 | 0.86 | 0.58 | 1.46 | 0.99 | 0.22 | 0.00 | 0.09 | 0.98 |
| phenyllactate | 3848 | 0.83 | 0.74 | 1.12 | 1 | 0.16 | 0.03 | 0.63 | 0.99 |
| (PLA) | | | | | | | | | |
| phenylacetate | 999 | 0.83 | 0.69 | 2.12 | 1.75 | 0.36 | 0.08 | 0.09 | 0.20 |
| 4- | 127 | 0.73 | 0.73 | 1.31 | 1.31 | 0.05 | 0.07 | 0.37 | 0.37 |
| hydroxyphenylacetate | | | | | | | | | |
| phenylacetylglutamine | 92258 | 1.1 | 0.76 | 1.41 | 0.98 | 0.58 | 0.13 | 0.27 | 0.95 |
| tyrosine | 6057 | 1.35 | 1.28 | 1.4 | 1.33 | 0.06 | 0.15 | 0.19 | 0.28 |
| N-acetyltyrosine | 68310 | 1.1 | 0.77 | 1.48 | 1.04 | 0.56 | 0.15 | 0.14 | 0.89 |
| tyramine | 5610 | 1.03 | 1.07 | 0.93 | 0.96 | 0.90 | 0.79 | 0.90 | 0.95 |
| 4- | 979 | 1.02 | 0.62 | 2.29 | 1.4 | 0.90 | 0.01 | 0.00 | 0.14 |
| hydroxyphenylpyruvate | | | | | | | | | |
| 3-(4- | 9378 | 0.81 | 0.83 | 1.05 | 1.07 | 0.12 | 0.18 | 0.82 | 0.77 |
| hydroxyphenyl)lactate | | | | | | | | | |
| phenol sulfate | 74426 | 0.92 | 0.79 | 1.94 | 1.68 | 0.45 | 0.06 | 0.02 | 0.07 |
| p-cresol sulfate | 4615423 | 1.18 | 0.83 | 1.99 | 1.39 | 0.36 | 0.32 | 0.02 | 0.26 |
| 3-(4- | 10394 | 0.67 | 0.47 | 1.25 | 0.88 | 0.05 | 0.00 | 0.46 | 0.67 |
| hydroxyphenyl)propionate | | | | | | | | | |
| 3- | 107 | 0.61 | 0.49 | 1.64 | 1.32 | 0.01 | 0.00 | 0.25 | 0.51 |
| phenylpropionate | | | | | | | | | |
| (hydrocinnamate) | | | | | | | | | |
| N- | 759256 | 1.05 | 0.84 | 0.36 | 0.29 | 0.67 | 0.19 | 0.06 | 0.02 |
| formylphenylalanine | | | | | | | | | |
| tryptophan | 6305 | 1.38 | 1.05 | 1.64 | 1.24 | 0.10 | 0.82 | 0.11 | 0.48 |
| N- | 700653 | 1.16 | 0.67 | 1.43 | 0.83 | 0.51 | 0.09 | 0.24 | 0.53 |
| acetyltryptophan | | | | | | | | | |
| indolelactate | 92904 | 0.92 | 0.7 | 1.31 | 0.99 | 0.65 | 0.06 | 0.36 | 0.98 |
| indoleacetate | 802 | 0.64 | 0.46 | 0.69 | 0.5 | 0.18 | 0.03 | 0.49 | 0.21 |
| indolepropionate | 3744 | 0.82 | 0.45 | 2.21 | 1.21 | 0.23 | 0.00 | 0.01 | 0.53 |
| 3-indoxyl sulfate | 10258 | 1.21 | 0.65 | 1.91 | 1.02 | 0.47 | 0.12 | 0.09 | 0.96 |
| kynurenine | 161166 | 1.02 | 0.8 | 1.63 | 1.27 | 0.90 | 0.26 | 0.07 | 0.37 |
| kynurenate | 3845 | 1.06 | 0.72 | 1.42 | 0.97 | 0.62 | 0.02 | 0.04 | 0.84 |
| tryptophan betaine | 442106 | 0.91 | 0.64 | 1.43 | 1 | 0.64 | 0.04 | 0.47 | 0.99 |
| C- | 1.1E+07 | 1.02 | 0.67 | 1.85 | 1.21 | 0.91 | 0.03 | 0.05 | 0.53 |
| glycosyltryptophan | | | | | | | | | |
| leucine | 6106 | 1.31 | 1.07 | 1.58 | 1.29 | 0.09 | 0.68 | 0.10 | 0.36 |
| N-acetylleucine | 70912 | 0.99 | 0.69 | 1.34 | 0.92 | 0.97 | 0.04 | 0.32 | 0.78 |
| 4-methyl-2- | 70 | 0.84 | 0.68 | 1.96 | 1.58 | 0.31 | 0.04 | 0.02 | 0.12 |
| oxopentanoate | | | | | | | | | |
| isovalerate | 10430 | 1.16 | 1.06 | 1.17 | 1.07 | 0.17 | 0.60 | 0.33 | 0.68 |
| isovalerylcarnitine | 6426851 | 1.21 | 0.96 | 1.09 | 0.86 | 0.38 | 0.85 | 0.84 | 0.72 |
| beta- | 69362 | 1.02 | 0.78 | 1.96 | 1.51 | 0.90 | 0.09 | 0.01 | 0.11 |
| hydroxyisovalerate | | | | | | | | | |
| beta- | | 0.91 | 0.83 | 1.25 | 1.15 | 0.51 | 0.23 | 0.24 | 0.47 |
| hydroxyisovaleroylcarnitine | | | | | | | | | |
| alpha- | 99823 | 0.92 | 0.68 | 1.8 | 1.34 | 0.59 | 0.03 | 0.05 | 0.32 |
| hydroxyisovalerate | | | | | | | | | |
| methylsuccinate | 10349 | 0.79 | 0.69 | 1.25 | 1.09 | 0.08 | 0.01 | 0.30 | 0.68 |
| isoleucine | 6306 | 1.56 | 1.26 | 1.62 | 1.31 | 0.03 | 0.27 | 0.14 | 0.41 |
| N-acetylisoleucine | 2802421 | 0.93 | 0.84 | 1.2 | 1.09 | 0.69 | 0.36 | 0.43 | 0.72 |
| 3-methyl-2- | 47 | 0.98 | 0.89 | 1.84 | 1.68 | 0.89 | 0.53 | 0.04 | 0.07 |
| oxovalerate | | | | | | | | | |
| 2- | 6426901 | 0.98 | 0.75 | 1.72 | 1.31 | 0.90 | 0.05 | 0.03 | 0.28 |
| methylbutyrylcarnitine (C5) | | | | | | | | | |
| 2-hydroxy-3- | 164623 | 1.09 | 0.71 | 1.85 | 1.21 | 0.62 | 0.06 | 0.06 | 0.54 |
| methylvalerate | | | | | | | | | |
| ethylmalonate | 11756 | 0.9 | 0.8 | 1.33 | 1.18 | 0.33 | 0.05 | 0.16 | 0.40 |
| valine | 6287 | 1.2 | 0.92 | 1.59 | 1.22 | 0.25 | 0.63 | 0.10 | 0.48 |
| N-acetylvaline | 66789 | 0.96 | 0.69 | 1.32 | 0.96 | 0.76 | 0.03 | 0.31 | 0.89 |
| 3-methyl-2- | 49 | 0.76 | 0.59 | 1.61 | 1.26 | 0.05 | 0.00 | 0.03 | 0.28 |
| oxobutyrate | | | | | | | | | |
| isobutyrylcarnitine | 168379 | 1.18 | 0.78 | 1.56 | 1.02 | 0.25 | 0.10 | 0.07 | 0.92 |
| 3- | 87 | 1.1 | 0.71 | 1.59 | 1.03 | 0.39 | 0.01 | 0.01 | 0.85 |
| hydroxyisobutyrate | | | | | | | | | |
| alpha- | 83697 | 0.89 | 0.8 | 1.38 | 1.24 | 0.47 | 0.20 | 0.28 | 0.47 |
| hydroxyisocaproate | | | | | | | | | |
| methionine | 6137 | 1.2 | 1.08 | 1.41 | 1.28 | 0.16 | 0.56 | 0.13 | 0.28 |
| N- | 448580 | 1.11 | 0.66 | 2.01 | 1.19 | 0.57 | 0.05 | 0.05 | 0.62 |
| acetylmethionine | | | | | | | | | |
| N- | 439750 | 1.06 | 0.57 | 2.38 | 1.28 | 0.81 | 0.04 | 0.01 | 0.47 |
| formylmethionine | | | | | | | | | |
| methionine | 158980 | 1.62 | 0.94 | 3.55 | 2.07 | 0.14 | 0.86 | 0.02 | 0.18 |
| sulfoxide | | | | | | | | | |
| N- | 193368 | 1.36 | 0.67 | 2.32 | 1.16 | 0.22 | 0.14 | 0.05 | 0.73 |
| acetylmethionine | | | | | | | | | |
| sulfoxide | | | | | | | | | |
| 2-aminobutyrate | 439691 | 1.02 | 0.9 | 1.12 | 0.99 | 0.73 | 0.13 | 0.33 | 0.90 |
| cystine | 67678 | 2.74 | 2.55 | 1.57 | 1.47 | 0.00 | 0.01 | 0.31 | 0.40 |
| S-methylcysteine | 24417 | 1 | 0.64 | 1.81 | 1.16 | 0.99 | 0.02 | 0.05 | 0.62 |
| cysteine s-sulfate | 115015 | 2.62 | 2.91 | 1.43 | 1.59 | 0.00 | 0.00 | 0.23 | 0.12 |
| cysteine sulfinic | 109 | 1.22 | 1.07 | 1.51 | 1.33 | 0.28 | 0.70 | 0.23 | 0.40 |
| acid | | | | | | | | | |
| hypotaurine | 107812 | 0.92 | 0.61 | 2.67 | 1.76 | 0.76 | 0.08 | 0.05 | 0.24 |
| taurine | 1123 | 0.98 | 0.78 | 1.54 | 1.22 | 0.90 | 0.06 | 0.07 | 0.39 |
| N-acetyltaurine | 159864 | 0.86 | 0.74 | 1.4 | 1.2 | 0.28 | 0.05 | 0.23 | 0.51 |
| 2- | | 0.87 | 0.76 | 1.75 | 1.52 | 0.28 | 0.04 | 0.01 | 0.06 |
| hydroxybutyrate/2- | | | | | | | | | |
| hydroxyisobutyrate | | | | | | | | | |
| arginine | 232 | 1.15 | 1.01 | 1.15 | 1.01 | 0.27 | 0.94 | 0.46 | 0.95 |
| urea | 1176 | 1.17 | 1.2 | 0.97 | 0.99 | 0.43 | 0.38 | 0.92 | 0.98 |
| ornithine | 6262 | 1.16 | 1.49 | 1.11 | 1.42 | 0.36 | 0.02 | 0.70 | 0.19 |
| proline | 145742 | 1.33 | 1.27 | 1.48 | 1.41 | 0.05 | 0.12 | 0.16 | 0.22 |
| citrulline | 9750 | 0.89 | 0.83 | 1.58 | 1.47 | 0.45 | 0.25 | 0.11 | 0.17 |
| argininosuccinate | 16950; 828 | 0.84 | 0.88 | 0.95 | 1 | 0.26 | 0.44 | 0.84 | 1.00 |
| homoarginine | 9085 | 0.72 | 0.69 | 1.43 | 1.35 | 0.06 | 0.04 | 0.20 | 0.27 |
| homocitrulline | 65072 | 0.95 | 0.94 | 1.24 | 1.22 | 0.72 | 0.65 | 0.41 | 0.45 |
| dimethylarginine | 123831 | 0.94 | 0.71 | 1.71 | 1.29 | 0.74 | 0.07 | 0.09 | 0.42 |
| (SDMA + ADMA) | | | | | | | | | |
| N-acetylarginine | 67427 | 0.81 | 0.79 | 1.55 | 1.52 | 0.23 | 0.20 | 0.11 | 0.13 |
| N-delta- | 9920500 | 0.8 | 0.69 | 1.37 | 1.18 | 0.08 | 0.01 | 0.17 | 0.48 |
| acetylornithine | | | | | | | | | |
| N2,N5- | 1E+07 | 0.98 | 0.82 | 1.17 | 0.98 | 0.87 | 0.15 | 0.54 | 0.94 |
| diacetylornithine | | | | | | | | | |
| N-methylproline | 557 | 1.11 | 1.4 | 1.22 | 1.54 | 0.69 | 0.22 | 0.59 | 0.24 |
| trans-4- | 5810 | 1.04 | 0.76 | 1.35 | 0.99 | 0.79 | 0.06 | 0.17 | 0.95 |
| hydroxyproline | | | | | | | | | |
| N-acetylcitrulline | 656979 | 0.86 | 0.48 | 1.75 | 0.98 | 0.40 | 0.00 | 0.06 | 0.93 |
| creatine | 586 | 1 | 0.83 | 1.37 | 1.13 | 1.00 | 0.12 | 0.17 | 0.58 |
| creatinine | 588 | 0.93 | 0.8 | 1.3 | 1.11 | 0.46 | 0.03 | 0.12 | 0.52 |
| guanidinoacetate | 763 | 0.97 | 0.86 | 1.3 | 1.14 | 0.82 | 0.21 | 0.23 | 0.54 |
| agmatine | 199 | 0.83 | 0.5 | 1.62 | 0.98 | 0.35 | 0.00 | 0.16 | 0.95 |
| acisoga | 129397 | 0.87 | 0.98 | 1.25 | 1.4 | 0.31 | 0.87 | 0.29 | 0.11 |
| putrescine | 1045 | 0.78 | 0.62 | 1.17 | 0.92 | 0.08 | 0.00 | 0.59 | 0.78 |
| spermidine | 1102 | 0.73 | 0.63 | 1.52 | 1.3 | 0.04 | 0.00 | 0.11 | 0.32 |
| 5- | 439176 | 1 | 0.66 | 2.42 | 1.6 | 0.99 | 0.09 | 0.00 | 0.12 |
| methylthioadenosine | | | | | | | | | |
| (MTA) | | | | | | | | | |
| N(1)- | 916 | 0.97 | 0.63 | 2.32 | 1.51 | 0.86 | 0.01 | 0.02 | 0.24 |
| acetylspermine | | | | | | | | | |
| N-acetylputreseine | 122356 | 0.72 | 0.63 | 1.39 | 1.22 | 0.01 | 0.00 | 0.20 | 0.43 |
| 4- | 500 | 0.97 | 0.78 | 1.29 | 1.03 | 0.84 | 0.10 | 0.34 | 0.91 |
| guanidinobutanoate | | | | | | | | | |
| guanidinosuccinate | 97856 | 1.19 | 0.74 | 1.17 | 0.73 | 0.27 | 0.07 | 0.63 | 0.34 |
| cys-gly, oxidized | 333293 | 0.39 | 0.17 | 2.78 | 1.19 | 0.01 | 0.00 | 0.01 | 0.65 |
| 5-oxoproline | 7405 | 1.04 | 0.86 | 1.35 | 1.12 | 0.71 | 0.20 | 0.16 | 0.60 |
| gamma- | 7017195 | 1.15 | 1.26 | 1.18 | 1.29 | 0.29 | 0.10 | 0.57 | 0.38 |
| glutamylhistidine | | | | | | | | | |
| gamma- | 1.4E+07 | 0.56 | 0.91 | 0.67 | 1.09 | 0.03 | 0.73 | 0.26 | 0.80 |
| glutamylisoleucine* | | | | | | | | | |
| gamma- | 151023 | 0.46 | 0.34 | 1.19 | 0.88 | 0.03 | 0.00 | 0.68 | 0.77 |
| glutamylleucine | | | | | | | | | |
| gamma-glutamyl- | 65254; 14284565 | 1.7 | 1.23 | 1.48 | 1.08 | 0.05 | 0.46 | 0.35 | 0.86 |
| epsilon-lysine | | | | | | | | | |
| gamma- | 7009567 | 1.03 | 1.28 | 0.6 | 0.75 | 0.89 | 0.23 | 0.09 | 0.33 |
| glutamylmethionine | | | | | | | | | |
| gamma- | 111299 | 0.8 | 0.82 | 1.1 | 1.13 | 0.12 | 0.18 | 0.74 | 0.68 |
| glutamylphenylalanine | | | | | | | | | |
| gamma- | 94340 | 0.94 | 0.39 | 2.19 | 0.91 | 0.78 | 0.00 | 0.02 | 0.77 |
| glutamyltyrosine | | | | | | | | | |
| gamma- | 7015683 | 1.25 | 0.84 | 1.8 | 1.2 | 0.38 | 0.51 | 0.14 | 0.64 |
| glutamylvaline | | | | | | | | | |
| carnosine | 439224 | 0.79 | 0.45 | 1.39 | 0.8 | 0.21 | 0.00 | 0.23 | 0.41 |
| anserine | 112072 | 0.59 | 0.45 | 1.7 | 1.27 | 0.05 | 0.00 | 0.15 | 0.51 |
| alanylleucine | 259583 | 0.57 | 0.23 | 1.98 | 0.81 | 0.06 | 0.00 | 0.07 | 0.57 |
| glycylisoleucine | 88079 | 0.91 | 0.65 | 2.17 | 1.55 | 0.65 | 0.05 | 0.03 | 0.22 |
| glycylleucine | 92843 | 0.99 | 0.69 | 1.91 | 1.34 | 0.96 | 0.13 | 0.08 | 0.43 |
| glycylvaline | 97417 | 1.16 | 0.91 | 2.02 | 1.57 | 0.46 | 0.64 | 0.07 | 0.23 |
| isoleucylglycine | 342532 | 0.88 | 0.49 | 1.81 | 1 | 0.47 | 0.00 | 0.03 | 1.00 |
| leucylglycine | 79070 | 0.73 | 0.45 | 1.82 | 1.11 | 0.16 | 0.00 | 0.04 | 0.71 |
| phenylalanylalanine | 6993123; 5488196 | 0.34 | 0.13 | 2.81 | 1.09 | 0.01 | 0.00 | 0.03 | 0.85 |
| phenylalanylglycine | 98207 | 0.7 | 0.31 | 1.77 | 0.79 | 0.22 | 0.00 | 0.07 | 0.47 |
| prolylglycine | 7408076; 626709 | 0.88 | 0.95 | 1.28 | 1.38 | 0.43 | 0.75 | 0.43 | 0.31 |
| threonylphenylalanine | 4099799; 4099798 | 0.33 | 0.13 | 2.25 | 0.86 | 0.01 | 0.00 | 0.07 | 0.74 |
| valylglutamine | 5253209 | 0.56 | 0.21 | 2.32 | 0.88 | 0.08 | 0.00 | 0.03 | 0.73 |
| valylglycine | 136487 | 0.87 | 0.55 | 1.78 | 1.14 | 0.52 | 0.01 | 0.07 | 0.69 |
| valylleucine | 352039 | 0.68 | 0.27 | 2.17 | 0.86 | 0.16 | 0.00 | 0.03 | 0.67 |
| leucylglutamine* | 4305457 | 0.41 | 0.14 | 2.96 | 1.05 | 0.04 | 0.00 | 0.03 | 0.92 |
| 1,5- | 64960 | 1.15 | 0.9 | 1.58 | 1.23 | 0.35 | 0.50 | 0.12 | 0.48 |
| anhydroglucitol | | | | | | | | | |
| (1,5-AG) | | | | | | | | | |
| glucose | 79025 | 0.86 | 0.62 | 1.56 | 1.12 | 0.40 | 0.02 | 0.15 | 0.70 |
| 2- | 59 | 0.97 | 0.77 | 1.32 | 1.04 | 0.78 | 0.02 | 0.31 | 0.88 |
| phosphoglycerate | | | | | | | | | |
| 3- | 724 | 1.09 | 0.87 | 0.94 | 0.75 | 0.44 | 0.26 | 0.75 | 0.17 |
| phosphoglycerate | | | | | | | | | |
| phosphoenolpyruvate | 1005 | 0.87 | 0.37 | 2.29 | 0.97 | 0.44 | 0.00 | 0.07 | 0.95 |
| (PEP) | | | | | | | | | |
| pyruvate | 1060 | 0.86 | 0.86 | 1.49 | 1.48 | 0.29 | 0.30 | 0.04 | 0.04 |
| lactate | 612 | 0.93 | 0.78 | 1.84 | 1.55 | 0.62 | 0.13 | 0.03 | 0.12 |
| glycerate | 752 | 0.79 | 0.67 | 1.49 | 1.27 | 0.15 | 0.03 | 0.20 | 0.44 |
| 6- | 91493 | 0.92 | 0.81 | 1.08 | 0.96 | 0.48 | 0.12 | 0.84 | 0.91 |
| phosphogluconate | | | | | | | | | |
| arabonate/xylonate | | 1.21 | 0.71 | 1.8 | 1.05 | 0.31 | 0.08 | 0.04 | 0.85 |
| ribose | 5779 | 0.81 | 0.6 | 1.68 | 1.24 | 0.24 | 0.01 | 0.15 | 0.54 |
| ribitol | 6912 | 0.82 | 0.73 | 1.35 | 1.21 | 0.14 | 0.03 | 0.28 | 0.49 |
| ribonate | 5460677 | 1.11 | 0.72 | 1.69 | 1.09 | 0.52 | 0.07 | 0.12 | 0.79 |
| fucose | 19466 | 0.66 | 0.72 | 1.07 | 1.17 | 0.00 | 0.01 | 0.75 | 0.46 |
| arabitol/xylitol | | 1.1 | 0.94 | 1.59 | 1.36 | 0.60 | 0.76 | 0.18 | 0.37 |
| maltotetraose | 446495 | 1.59 | 0.69 | 2.35 | 1.01 | 0.14 | 0.26 | 0.07 | 0.98 |
| maltotriose | 439586 | 1.11 | 0.58 | 1.9 | 1 | 0.77 | 0.16 | 0.16 | 1.00 |
| maltose | 1.1E+07 | 1.1 | 0.97 | 0.84 | 0.74 | 0.68 | 0.90 | 0.75 | 0.59 |
| Lewis X | 4571095 | 1.12 | 1.1 | 1.37 | 1.35 | 0.64 | 0.70 | 0.42 | 0.44 |
| trisaccharide | | | | | | | | | |
| sucrose | 5988 | 2.39 | 1.64 | 0.88 | 0.6 | 0.02 | 0.19 | 0.76 | 0.23 |
| fructose | 5984 | 1 | 0.56 | 2.18 | 1.22 | 1.00 | 0.09 | 0.16 | 0.71 |
| mannitol/sorbitol | 5780 | 0.52 | 0.13 | 2.22 | 0.58 | 0.12 | 0.00 | 0.14 | 0.30 |
| mannose | 18950 | 0.97 | 0.75 | 1.06 | 0.82 | 0.87 | 0.17 | 0.80 | 0.40 |
| galactonate | 128869 | 0.96 | 0.76 | 1.11 | 0.88 | 0.85 | 0.21 | 0.68 | 0.60 |
| glucuronate | 444791 | 1.2 | 0.88 | 1.52 | 1.11 | 0.20 | 0.39 | 0.16 | 0.72 |
| N- | 439197 | 0.69 | 0.97 | 1.11 | 1.56 | 0.01 | 0.83 | 0.71 | 0.11 |
| acetylneuraminate | | | | | | | | | |
| N-acetylmuramate | 5462244 | 0.74 | 0.56 | 1.75 | 1.33 | 0.17 | 0.02 | 0.14 | 0.45 |
| erythronate* | 2781043 | 1.07 | 0.82 | 1.46 | 1.12 | 0.64 | 0.18 | 0.12 | 0.63 |
| citrate | 311 | 1.33 | 1.01 | 1.25 | 0.95 | 0.04 | 0.92 | 0.35 | 0.83 |
| isocitrate | 1198 | 1.08 | 0.91 | 1.05 | 0.88 | 0.46 | 0.39 | 0.74 | 0.34 |
| alpha- | 51 | 0.73 | 0.66 | 1.42 | 1.27 | 0.01 | 0.00 | 0.09 | 0.24 |
| ketoglutarate | | | | | | | | | |
| succinylcarnitine | | 0.95 | 0.71 | 1.58 | 1.17 | 0.77 | 0.06 | 0.14 | 0.61 |
| succinate | 1110 | 0.63 | 0.52 | 1.4 | 1.17 | 0.01 | 0.00 | 0.24 | 0.59 |
| fumarate | 444972 | 1.1 | 0.73 | 1.59 | 1.06 | 0.60 | 0.09 | 0.04 | 0.78 |
| malate | 525 | 0.92 | 0.73 | 1.68 | 1.33 | 0.51 | 0.03 | 0.02 | 0.20 |
| tricarballylate | 14925 | 1.04 | 0.66 | 1.55 | 0.99 | 0.81 | 0.01 | 0.24 | 0.97 |
| phosphate | 1061 | 0.78 | 0.7 | 0.89 | 0.81 | 0.17 | 0.07 | 0.73 | 0.52 |
| 2- | 43 | 0.87 | 0.73 | 1.3 | 1.09 | 0.22 | 0.01 | 0.25 | 0.69 |
| hydroxyglutarate | | | | | | | | | |
| maleate | 444266 | 1.35 | 1 | 0.9 | 0.67 | 0.03 | 0.98 | 0.62 | 0.05 |
| 3-carboxy-4- | 123979 | 0.97 | 1.03 | 0.99 | 1.05 | 0.31 | 0.34 | 0.84 | 0.37 |
| methyl-5-propyl- | | | | | | | | | |
| 2-furanpropanoate | | | | | | | | | |
| (CMPF) | | | | | | | | | |
| butyrylcarnitine | 439829 | 1.01 | 0.83 | 1.96 | 1.6 | 0.94 | 0.27 | 0.02 | 0.10 |
| propionylcarnitine | 107738 | 0.96 | 0.79 | 1.43 | 1.18 | 0.73 | 0.10 | 0.16 | 0.51 |
| methylmalonate | 487 | 0.91 | 0.71 | 1.44 | 1.13 | 0.49 | 0.02 | 0.13 | 0.62 |
| (MMA) | | | | | | | | | |
| acetylcarnitine | 1 | 0.94 | 0.85 | 1.44 | 1.3 | 0.69 | 0.29 | 0.17 | 0.33 |
| 3- | 5.3E+07 | 0.79 | 0.61 | 1.28 | 0.99 | 0.12 | 0.00 | 0.29 | 0.98 |
| hydroxybutyrylcarnitine | | | | | | | | | |
| (1) | | | | | | | | | |
| hexanoylcarnitine | 6426853 | 1.07 | 0.95 | 1.74 | 1.54 | 0.56 | 0.68 | 0.02 | 0.07 |
| octanoylcarnitine | 123701 | 1.22 | 1.23 | 1.41 | 1.43 | 0.20 | 0.20 | 0.11 | 0.10 |
| deoxycarnitine | 134 | 0.9 | 0.68 | 1.33 | 1 | 0.37 | 0.00 | 0.31 | 0.99 |
| carnitine | 10917 | 0.95 | 0.8 | 1.5 | 1.26 | 0.65 | 0.06 | 0.04 | 0.23 |
| 3-hydroxybutyrate | 441 | 0.91 | 0.7 | 1.37 | 1.05 | 0.41 | 0.00 | 0.10 | 0.80 |
| (BHBA) | | | | | | | | | |
| 4-hydroxybutyrate | 10413 | 1.09 | 0.85 | 1.3 | 1.02 | 0.33 | 0.09 | 0.09 | 0.90 |
| (GHB) | | | | | | | | | |
| 13-HODE + 9- | 43013 | 1.07 | 0.67 | 1.75 | 1.1 | 0.72 | 0.04 | 0.04 | 0.73 |
| HODE | | | | | | | | | |
| myo-inositol | 892 | 1.16 | 0.86 | 1.49 | 1.11 | 0.41 | 0.43 | 0.18 | 0.71 |
| choline | 305 | 0.89 | 0.72 | 1.35 | 1.1 | 0.31 | 0.01 | 0.20 | 0.67 |
| choline phosphate | 1014 | 3.19 | 2 | 1.04 | 0.65 | 0.02 | 0.16 | 0.96 | 0.60 |
| glycerophosphorylcholine | 71920 | 0.97 | 1.24 | 1.2 | 1.53 | 0.80 | 0.11 | 0.49 | 0.11 |
| (GPC) | | | | | | | | | |
| phosphoethanolamine | 1015 | 2.47 | 1.64 | 1.21 | 0.8 | 0.01 | 0.13 | 0.73 | 0.69 |
| trimethylamine N- | 1145 | 0.79 | 0.45 | 1.1 | 0.63 | 0.45 | 0.02 | 0.86 | 0.38 |
| oxide | | | | | | | | | |
| glycerophosphoinositol* | | 1.04 | 1.1 | 1.02 | 1.07 | 0.46 | 0.14 | 0.80 | 0.36 |
| glycerol | 753 | 1.17 | 1.04 | 1.34 | 1.19 | 0.38 | 0.84 | 0.32 | 0.55 |
| glycerol 3- | 754 | 1.58 | 1.1 | 1.32 | 0.92 | 0.08 | 0.71 | 0.54 | 0.86 |
| phosphate | | | | | | | | | |
| palmitoyl sphingomyelin | 9939941 | 0.72 | 0.77 | 0.94 | 0.99 | 0.03 | 0.09 | 0.72 | 0.98 |
| (d18:1/16:0) | | | | | | | | | |
| 3-hydroxy-3- | 1662 | 0.91 | 0.76 | 1.17 | 0.97 | 0.50 | 0.06 | 0.49 | 0.89 |
| methylglutarate | | | | | | | | | |
| mevalonate | 439230 | 0.91 | 0.69 | 1.45 | 1.1 | 0.49 | 0.01 | 0.06 | 0.62 |
| mevalonolactone | 10428 | 1.08 | 0.79 | 1.45 | 1.07 | 0.74 | 0.32 | 0.13 | 0.79 |
| inosine | 6021 | 0.55 | 0.55 | 0.5 | 0.5 | 0.00 | 0.00 | 0.01 | 0.01 |
| hypoxanthine | 790 | 0.91 | 0.75 | 1.99 | 1.65 | 0.56 | 0.10 | 0.02 | 0.10 |
| xanthine | 1188 | 0.94 | 0.76 | 1.83 | 1.47 | 0.67 | 0.07 | 0.05 | 0.20 |
| xanthosine | 64959 | 0.77 | 0.41 | 1.54 | 0.82 | 0.18 | 0.00 | 0.17 | 0.51 |
| 2′-deoxyinosine | 65058 | 1.22 | 0.84 | 0.99 | 0.68 | 0.42 | 0.49 | 0.98 | 0.28 |
| urate | 1175 | 1.01 | 0.95 | 1.53 | 1.44 | 0.96 | 0.67 | 0.06 | 0.10 |
| allantoin | 204 | 0.81 | 0.81 | 1.15 | 1.15 | 0.23 | 0.25 | 0.66 | 0.65 |
| adenosine | 60961 | 0.79 | 0.91 | 0.39 | 0.45 | 0.23 | 0.63 | 0.00 | 0.00 |
| adenine | 190 | 2.88 | 2.55 | 1.27 | 1.13 | 0.00 | 0.00 | 0.45 | 0.70 |
| 1-methyladenine | 78821 | 0.92 | 0.71 | 1.73 | 1.34 | 0.56 | 0.04 | 0.05 | 0.29 |
| N1- | 27476 | 1 | 1.08 | 0.89 | 0.97 | 0.99 | 0.74 | 0.71 | 0.93 |
| methyladenosine | | | | | | | | | |
| N6- | 161466 | 1.15 | 0.76 | 1.35 | 0.89 | 0.32 | 0.06 | 0.19 | 0.62 |
| carbamoylthreonyl adenosine | | | | | | | | | |
| 2′-deoxyadenosine | 13730 | 1.18 | 0.9 | 0.8 | 0.62 | 0.28 | 0.52 | 0.34 | 0.04 |
| N6- | | 1.01 | 0.8 | 1.35 | 1.08 | 0.97 | 0.34 | 0.33 | 0.81 |
| succinyladenosine | | | | | | | | | |
| guanosine | 6802 | 0.47 | 0.67 | 0.25 | 0.36 | 0.01 | 0.20 | 0.00 | 0.01 |
| guanine | 764 | 0.92 | 0.98 | 0.44 | 0.47 | 0.68 | 0.92 | 0.05 | 0.07 |
| 7-methylguanine | 11361 | 0.89 | 0.72 | 1.36 | 1.1 | 0.36 | 0.02 | 0.19 | 0.69 |
| N2,N2- | 92919 | 1.14 | 0.61 | 1.77 | 0.94 | 0.55 | 0.03 | 0.05 | 0.84 |
| dimethylguanosine | | | | | | | | | |
| N2,N2- | 74047 | 0.98 | 0.68 | 1.83 | 1.26 | 0.91 | 0.03 | 0.06 | 0.46 |
| dimethylguanine | | | | | | | | | |
| 2′-deoxyguanosine | 187790 | 1.21 | 1.05 | 0.59 | 0.51 | 0.29 | 0.80 | 0.04 | 0.01 |
| orotate | 967 | 0.6 | 0.62 | 1.56 | 1.62 | 0.00 | 0.00 | 0.11 | 0.08 |
| orotidine | 92751 | 0.95 | 0.88 | 1.18 | 1.09 | 0.55 | 0.14 | 0.21 | 0.52 |
| uridine | 6029 | 0.93 | 1.67 | 0.54 | 0.97 | 0.71 | 0.02 | 0.04 | 0.93 |
| uracil | 1174 | 0.8 | 0.67 | 1.91 | 1.6 | 0.16 | 0.02 | 0.05 | 0.15 |
| pseudouridine | 15047 | 0.9 | 0.73 | 1.66 | 1.35 | 0.43 | 0.03 | 0.05 | 0.24 |
| 5-methyluridine | 445408 | 0.98 | 0.66 | 1.37 | 0.93 | 0.89 | 0.02 | 0.23 | 0.77 |
| (ribothymidine) | | | | | | | | | |
| 5,6-dihydrouracil | 649 | 0.9 | 0.79 | 1.21 | 1.06 | 0.37 | 0.05 | 0.32 | 0.75 |
| 2′-deoxyuridine | 13712 | 0.83 | 0.68 | 1.46 | 1.21 | 0.26 | 0.04 | 0.23 | 0.54 |
| beta-alanine | 239 | 0.71 | 0.7 | 1.3 | 1.28 | 0.01 | 0.01 | 0.26 | 0.29 |
| cytidine | 6175 | 0.88 | 0.35 | 0.58 | 0.23 | 0.75 | 0.02 | 0.34 | 0.01 |
| cytosine | 597 | 1.16 | 0.97 | 1.55 | 1.3 | 0.43 | 0.89 | 0.16 | 0.41 |
| 2′-deoxycytidine | 13711 | 1.41 | 0.75 | 1.03 | 0.55 | 0.08 | 0.16 | 0.94 | 0.07 |
| thymidine | 5789 | 1.05 | 0.69 | 1.13 | 0.74 | 0.74 | 0.02 | 0.70 | 0.33 |
| thymine | 1135 | 0.94 | 0.73 | 1.41 | 1.09 | 0.68 | 0.04 | 0.27 | 0.78 |
| 5,6- | 93556 | 0.9 | 0.73 | 1.29 | 1.04 | 0.36 | 0.01 | 0.17 | 0.82 |
| dihydrothymine | | | | | | | | | |
| 3- | 64956 | 0.93 | 0.73 | 1.31 | 1.03 | 0.54 | 0.01 | 0.23 | 0.90 |
| aminoisobutyrate | | | | | | | | | |
| nicotinate | 938 | 0.75 | 0.61 | 1.97 | 1.6 | 0.06 | 0.00 | 0.02 | 0.09 |
| nicotinate | 161234 | 1.47 | 0.82 | 2.86 | 1.59 | 0.27 | 0.59 | 0.03 | 0.32 |
| ribonucleoside | | | | | | | | | |
| nicotinamide | 936 | 0.54 | 0.47 | 0.63 | 0.55 | 0.10 | 0.06 | 0.33 | 0.21 |
| 1- | 1E+07 | 0.96 | 1.03 | 1.04 | 1.12 | 0.66 | 0.81 | 0.85 | 0.59 |
| methylnicotinamide | | | | | | | | | |
| trigonelline (N′- | 5570 | 0.64 | 0.55 | 1.34 | 1.15 | 0.07 | 0.03 | 0.49 | 0.74 |
| methylnicotinate) | | | | | | | | | |
| N1-Methyl-2- | 69698 | 0.93 | 0.88 | 1.7 | 1.61 | 0.50 | 0.27 | 0.03 | 0.04 |
| pyridone-5- | | | | | | | | | |
| carboxamide | | | | | | | | | |
| riboflavin | 493570 | 0.89 | 0.58 | 1.59 | 1.03 | 0.44 | 0.00 | 0.10 | 0.91 |
| (Vitamin B2) | | | | | | | | | |
| pantothenate | 6613 | 0.99 | 0.76 | 1.67 | 1.28 | 0.93 | 0.07 | 0.06 | 0.35 |
| threonate | 151152 | 1.38 | 0.83 | 1.87 | 1.12 | 0.06 | 0.27 | 0.05 | 0.70 |
| oxalate | 971 | 1.11 | 0.9 | 1.1 | 0.88 | 0.30 | 0.31 | 0.64 | 0.53 |
| (ethanedioate) | | | | | | | | | |
| gulonic acid* | 9794176 | 1.14 | 0.63 | 1.81 | 1 | 0.52 | 0.03 | 0.13 | 1.00 |
| 5-aminolevulinate | 137 | 0.88 | 0.74 | 1.05 | 0.88 | 0.20 | 0.00 | 0.76 | 0.47 |
| thiamin (Vitamin | 1130 | 0.85 | 0.75 | 1.23 | 1.09 | 0.27 | 0.07 | 0.45 | 0.76 |
| B1) | | | | | | | | | |
| pyridoxamine | 1052 | 0.83 | 0.6 | 1.25 | 0.9 | 0.10 | 0.00 | 0.26 | 0.60 |
| pyridoxal | 1050 | 0.85 | 0.64 | 1.65 | 1.24 | 0.26 | 0.00 | 0.06 | 0.42 |
| pyridoxate | 6723 | 0.95 | 1.05 | 0.87 | 0.96 | 0.65 | 0.69 | 0.56 | 0.85 |
| hippurate | 464 | 0.85 | 0.83 | 1.1 | 1.08 | 0.25 | 0.22 | 0.75 | 0.80 |
| 2- | 10253 | 1.13 | 1.17 | 1.23 | 1.27 | 0.27 | 0.17 | 0.50 | 0.43 |
| hydroxyhippurate | | | | | | | | | |
| (salicylurate) | | | | | | | | | |
| 3- | 450268 | 1.16 | 0.93 | 1.41 | 1.14 | 0.24 | 0.61 | 0.25 | 0.67 |
| hydroxyhippurate | | | | | | | | | |
| 4- | 151012 | 1.2 | 0.9 | 1.2 | 0.9 | 0.10 | 0.35 | 0.32 | 0.54 |
| hydroxyhippurate | | | | | | | | | |
| catechol sulfate | 3083879 | 1.25 | 0.87 | 1.72 | 1.2 | 0.36 | 0.60 | 0.22 | 0.68 |
| O-methylcatechol | 22473 | 1.11 | 1.02 | 1.11 | 1.03 | 0.27 | 0.83 | 0.26 | 0.77 |
| sulfate | | | | | | | | | |
| 4-methylcatechol | | 1.2 | 0.94 | 1.36 | 1.07 | 0.06 | 0.56 | 0.04 | 0.67 |
| sulfate | | | | | | | | | |
| caffeine | 2519 | 0.58 | 0.89 | 1.88 | 2.87 | 0.01 | 0.60 | 0.11 | 0.01 |
| paraxanthine | 4687 | 0.88 | 1.24 | 1.44 | 2.02 | 0.58 | 0.37 | 0.36 | 0.08 |
| theobromine | 5429 | 0.83 | 0.94 | 1.22 | 1.38 | 0.22 | 0.69 | 0.51 | 0.29 |
| theophylline | 2153 | 1.05 | 1.05 | 1.79 | 1.8 | 0.74 | 0.73 | 0.05 | 0.05 |
| 1-methylurate | 69726 | 1.12 | 0.79 | 1.91 | 1.35 | 0.55 | 0.23 | 0.10 | 0.44 |
| 7-methylurate | 69160 | 0.89 | 0.71 | 1.02 | 0.81 | 0.50 | 0.07 | 0.97 | 0.62 |
| 1,3-dimethylurate | 70346 | 1.02 | 0.92 | 1.19 | 1.07 | 0.85 | 0.36 | 0.25 | 0.63 |
| 1,7-dimethylurate | 91611 | 0.82 | 0.73 | 1.71 | 1.53 | 0.21 | 0.06 | 0.26 | 0.37 |
| 3,7-dimethylurate | 83126 | 1.06 | 0.9 | 1.09 | 0.92 | 0.37 | 0.11 | 0.44 | 0.46 |
| 1,3,7- | 79437 | 0.97 | 0.98 | 1 | 1 | 0.29 | 0.36 | 0.92 | 1.00 |
| trimethylurate | | | | | | | | | |
| 1-methylxanthine | 80220 | 0.86 | 0.74 | 1.53 | 1.32 | 0.40 | 0.13 | 0.24 | 0.44 |
| 3-methylxanthine | 70639 | 0.87 | 0.88 | 0.97 | 0.98 | 0.32 | 0.40 | 0.89 | 0.95 |
| 7-methylxanthine | 68374 | 1.03 | 0.85 | 1.02 | 0.84 | 0.91 | 0.46 | 0.96 | 0.61 |
| 5-acetylamino-6- | 88299 | 1.02 | 0.79 | 1.21 | 0.94 | 0.90 | 0.12 | 0.48 | 0.82 |
| amino-3- | | | | | | | | | |
| methyluracil | | | | | | | | | |
| cotinine | 854019 | 1.12 | 1 | 0.73 | 0.65 | 0.01 | 1.00 | 0.14 | 0.05 |
| hydroxycotinine | 1E+07 | 1.08 | 1 | 0.66 | 0.62 | 0.01 | 1.00 | 0.08 | 0.04 |
| 2-piperidinone | 12665 | 0.81 | 0.62 | 1.71 | 1.31 | 0.20 | 0.01 | 0.09 | 0.38 |
| 2,3- | 677 | 1 | 0.95 | 1.38 | 1.3 | 1.00 | 0.68 | 0.19 | 0.28 |
| dihydroxyisovalerate | | | | | | | | | |
| 2-isopropylmalate | 77 | 0.7 | 0.66 | 1.42 | 1.34 | 0.03 | 0.02 | 0.20 | 0.29 |
| 2-oxindole-3- | 3080590 | 0.81 | 0.6 | 0.83 | 0.62 | 0.35 | 0.03 | 0.61 | 0.18 |
| acetate | | | | | | | | | |
| betonicine | 164642 | 1.04 | 1.1 | 1.36 | 1.44 | 0.87 | 0.71 | 0.34 | 0.27 |
| gluconate | 10690 | 1.59 | 1.1 | 2.29 | 1.58 | 0.05 | 0.71 | 0.07 | 0.32 |
| ergothioneine | 3032311 | 1 | 0.72 | 1.38 | 0.99 | 0.98 | 0.01 | 0.20 | 0.98 |
| erythritol | 222285 | 0.77 | 0.47 | 1.67 | 1.02 | 0.32 | 0.01 | 0.21 | 0.97 |
| homostachydrine* | 441447 | 1.01 | 0.77 | 1.22 | 0.93 | 0.97 | 0.10 | 0.31 | 0.73 |
| piperine | 638024 | 1.03 | 0.91 | 1.1 | 0.97 | 0.87 | 0.64 | 0.76 | 0.92 |
| quinate | 6508 | 0.76 | 0.74 | 0.97 | 0.94 | 0.41 | 0.38 | 0.95 | 0.90 |
| saccharin | 5143 | 2.09 | 1.6 | 1.31 | 1.01 | 0.03 | 0.17 | 0.58 | 0.99 |
| stachydrine | 115244 | 1.03 | 0.7 | 2.16 | 1.46 | 0.92 | 0.33 | 0.15 | 0.47 |
| tartarate | 444305 | 1.63 | 1.05 | 1 | 0.65 | 0.08 | 0.85 | 1.00 | 0.12 |
| pyrraline | | 0.84 | 0.71 | 1.08 | 0.92 | 0.23 | 0.04 | 0.73 | 0.73 |
| 2- | | 1.13 | 0.95 | 1.07 | 0.9 | 0.33 | 0.69 | 0.70 | 0.55 |
| hydroxyacetaminophen | | | | | | | | | |
| sulfate* | | | | | | | | | |
| 4-acetaminophen | 83939 | 1.28 | 0.75 | 1.21 | 0.71 | 0.15 | 0.12 | 0.52 | 0.23 |
| sulfate | | | | | | | | | |
| 4- | 1983 | 1.28 | 0.52 | 1.38 | 0.56 | 0.36 | 0.02 | 0.47 | 0.20 |
| acetamidophenol | | | | | | | | | |
| 4- | 83944 | 1.02 | 1.04 | 1 | 1.02 | 0.45 | 0.24 | 1.00 | 0.62 |
| acetamidophenylglucuronide | | | | | | | | | |
| O- | | 0.84 | 1 | 0.38 | 0.45 | 0.24 | 1.00 | 0.05 | 0.10 |
| desmethylvenlafaxine | | | | | | | | | |
| dextromethorphan | 5362449 | 1.32 | 1 | 0.95 | 0.72 | 0.11 | 1.00 | 0.76 | 0.08 |
| diphenhydramine | 3100 | 1.19 | 1.05 | 0.62 | 0.55 | 0.06 | 0.64 | 0.14 | 0.06 |
| escitalopram | 146570 | 0.96 | 1 | 0.83 | 0.87 | 0.06 | 1.00 | 0.27 | 0.39 |
| hydroxybupropion | 446 | 0.93 | 1.11 | 0.83 | 0.99 | 0.41 | 0.25 | 0.30 | 0.94 |
| metformin | 4091 | 0.9 | 1 | 0.54 | 0.6 | 0.15 | 1.00 | 0.15 | 0.22 |
| metoprolol | 4171 | 0.93 | 1 | 0.76 | 0.82 | 0.46 | 1.00 | 0.20 | 0.33 |
| metoprolol acid | 62936 | 0.94 | 1 | 0.88 | 0.93 | 0.36 | 1.00 | 0.14 | 0.42 |
| metabolite* | | | | | | | | | |
| nicotine | 89594 | 1.09 | 0.69 | 0.79 | 0.5 | 0.56 | 0.02 | 0.49 | 0.05 |
| oxypurinol | 4644 | 1 | 1 | 1 | 1 | 1.00 | 0.15 | 1.00 | 0.14 |
| pseudoephedrine | 7028 | 0.98 | 1 | 0.98 | 1 | 0.18 | 1.00 | 0.18 | 1.00 |
| salicylate | 338 | 1.09 | 1.03 | 1.4 | 1.33 | 0.74 | 0.92 | 0.31 | 0.39 |
| venlafaxine | 5656 | 0.92 | 1 | 0.86 | 0.94 | 0.06 | 1.00 | 0.12 | 0.49 |
| 2-pyrrolidinone | 12025 | 1.29 | 0.88 | 1.22 | 0.83 | 0.13 | 0.46 | 0.37 | 0.39 |
| sulfate* | 1118 | 1.17 | 1.03 | 1.38 | 1.21 | 0.26 | 0.85 | 0.13 | 0.36 |
| O-sulfo-L-tyrosine | 514186 | 1.04 | 0.97 | 1.96 | 1.83 | 0.82 | 0.87 | 0.13 | 0.17 |
| dexpanthenol | 4678 | 0.93 | 1.36 | 0.79 | 1.16 | 0.62 | 0.06 | 0.43 | 0.63 |
| succinimide | 11439 | 1.24 | 0.93 | 1.52 | 1.13 | 0.26 | 0.70 | 0.16 | 0.67 |
| triethanolamine | 7618 | 1.03 | 1.04 | 1.58 | 1.59 | 0.89 | 0.87 | 0.34 | 0.33 |
| N- | | 0.92 | 0.9 | 1.14 | 1.11 | 0.52 | 0.40 | 0.58 | 0.67 |
| methylpipecolate | | | | | | | | | |
| 3-hydroxypyridine | | 1.15 | 1.29 | 1.89 | 2.13 | 0.65 | 0.42 | 0.19 | 0.12 |
| sulfate | | | | | | | | | |
| X - 11381 | | 0.97 | 0.77 | 1.26 | 0.99 | 0.81 | 0.05 | 0.37 | 0.98 |
| X - 12100 | | 1.16 | 0.79 | 1.5 | 1.03 | 0.38 | 0.20 | 0.10 | 0.90 |
| X - 12472 | | 1.1 | 0.72 | 1.85 | 1.2 | 0.49 | 0.03 | 0.00 | 0.37 |
| X - 12565 | | 0.92 | 0.88 | 0.69 | 0.66 | 0.66 | 0.53 | 0.17 | 0.13 |
| X - 12688 | | 0.85 | 0.64 | 1.33 | 1 | 0.29 | 0.01 | 0.35 | 1.00 |
| X - 12748 | | 1.36 | 0.49 | 2.86 | 1.04 | 0.23 | 0.01 | 0.02 | 0.93 |
| X - 12855 - retired | | 0.87 | 0.66 | 1.59 | 1.2 | 0.37 | 0.01 | 0.08 | 0.48 |
| for 3- | | | | | | | | | |
| hydroxybutyrylcarnitine (2) | | | | | | | | | |
| X - 13255 | | 0.8 | 0.94 | 0.91 | 1.06 | 0.06 | 0.60 | 0.50 | 0.67 |
| X - 13848 | | 0.69 | 0.18 | 6.65 | 1.74 | 0.37 | 0.00 | 0.01 | 0.41 |
| X - 14113 | | 2.02 | 2.18 | 1.57 | 1.69 | 0.01 | 0.01 | 0.30 | 0.22 |
| X - 14141 | | 1.29 | 0.98 | 1.95 | 1.48 | 0.40 | 0.95 | 0.09 | 0.31 |
| X - 14196 | | 1.57 | 0.86 | 2.14 | 1.18 | 0.03 | 0.48 | 0.01 | 0.53 |
| X - 14314 | | 1.14 | 0.7 | 2.13 | 1.32 | 0.55 | 0.13 | 0.02 | 0.40 |
| X - 14568 | | 1.08 | 0.83 | 1.37 | 1.05 | 0.62 | 0.24 | 0.28 | 0.87 |
| X - 14697 | | 1.19 | 0.84 | 2.08 | 1.46 | 0.44 | 0.46 | 0.10 | 0.39 |
| X - 16071 | | 0.68 | 0.5 | 2.06 | 1.54 | 0.04 | 0.00 | 0.09 | 0.31 |
| X - 17299 | | 0.94 | 0.76 | 1.63 | 1.33 | 0.65 | 0.07 | 0.07 | 0.29 |
| X - 18278 | | 0.34 | 0.22 | 0.67 | 0.43 | 0.02 | 0.00 | 0.42 | 0.09 |
| X - 21365 | | 0.9 | 0.73 | 1.18 | 0.95 | 0.29 | 0.00 | 0.44 | 0.83 |
| X - 21729 | | 1.21 | 1.13 | 1.64 | 1.53 | 0.15 | 0.39 | 0.19 | 0.26 |
| X - 21772 | | 1.14 | 1 | 1 | 0.87 | 0.18 | 1.00 | 1.00 | 0.18 |
| X - 23644 | | 1.03 | 0.58 | 1.27 | 0.72 | 0.93 | 0.08 | 0.58 | 0.44 |
| X - 23662 | | 0.99 | 0.69 | 1.35 | 0.94 | 0.96 | 0.03 | 0.32 | 0.84 |
| X - 23670 - retired | | 0.71 | 0.54 | 1.7 | 1.3 | 0.10 | 0.01 | 0.18 | 0.50 |
| for N1,N12- | | | | | | | | | |
| diacetylspermine | | | | | | | | | |
| X - 23673 | | 1.47 | 1 | 1 | 0.68 | 0.07 | 1.00 | 1.00 | 0.07 |
| X - 23747 | | 0.79 | 0.63 | 1.47 | 1.18 | 0.18 | 0.02 | 0.26 | 0.62 |
| X - 23775 | | 1.37 | 1.6 | 1.74 | 2.03 | 0.15 | 0.04 | 0.12 | 0.05 |
| X - 24020 | | 0.81 | 0.58 | 1.67 | 1.2 | 0.26 | 0.01 | 0.17 | 0.62 |
| X - 24071 | | 1.1 | 0.82 | 1.78 | 1.33 | 0.60 | 0.33 | 0.06 | 0.36 |
| X - 24240 | | 0.88 | 0.64 | 1.72 | 1.26 | 0.49 | 0.03 | 0.14 | 0.53 |
| X - 24243 | | 0.98 | 0.69 | 1.36 | 0.96 | 0.84 | 0.01 | 0.20 | 0.87 |
| X - 24246 | | 0.74 | 0.57 | 1.38 | 1.07 | 0.06 | 0.00 | 0.28 | 0.82 |
| X - 24529 | | 1.28 | 1.29 | 0.98 | 0.99 | 0.40 | 0.40 | 0.95 | 0.98 |

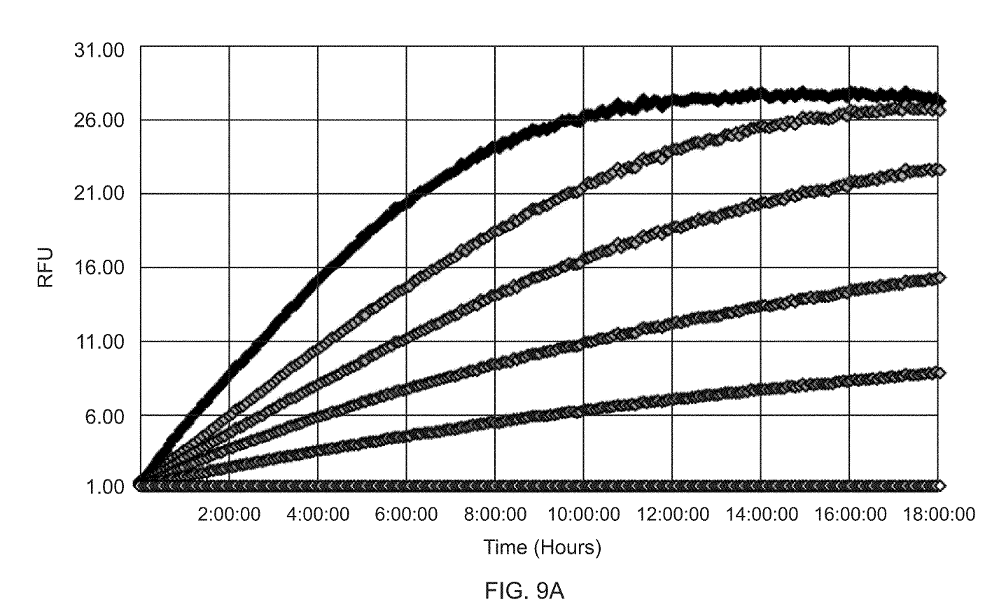

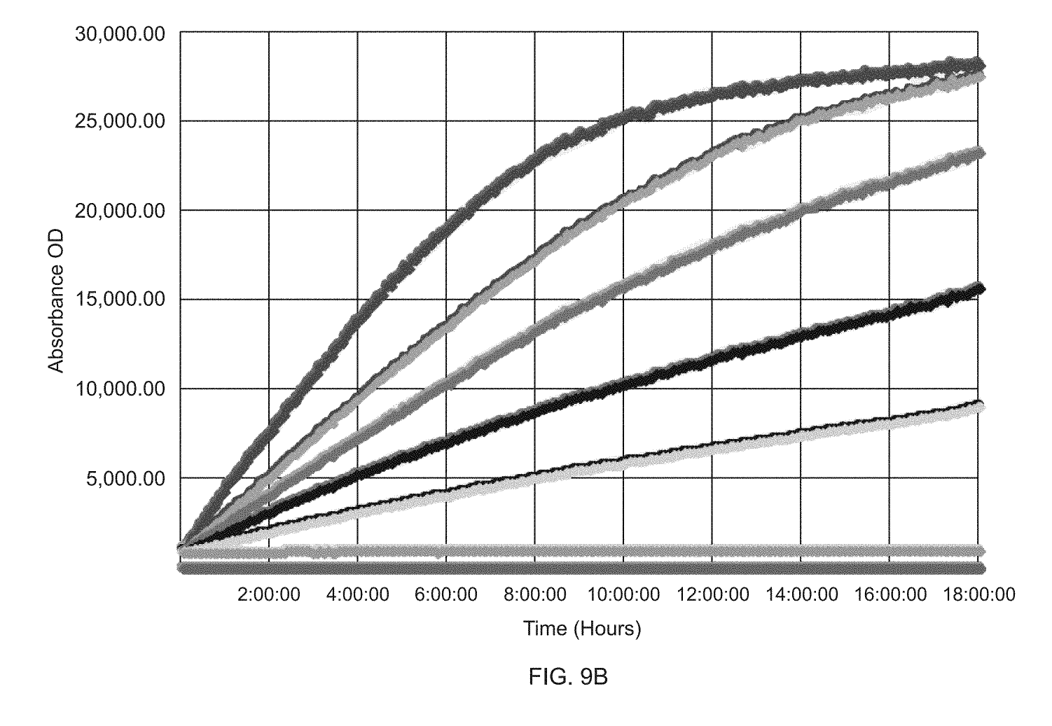

Another clinical study was carried out to examine the efficacy of ProHealth® toothpaste in treating gingivitis. This was a controlled, examiner-blind study. Sixty panelists were enrolled. Panelists had more than 20 bleeding sites and at least three dental pockets greater than or equal to 3 mM, but not deeper than 4 mM in depth. And the panelists also had three dental sites that were less than or equal to 2 mM deep without bleeding. Three bleeding and three non-bleeding sites were sampled for both supragingival and subgingival plaques. ProHealth® toothpaste was used by the panelists for 8 weeks, twice a day. Supragingival, subgingival plaques, and oral lavage were collected at baseline, week 4 and week 8 of the treatment. Oral lavage samples of the week 8 were pooled from the 60 panelists, labeled as pooled oral lavage samples. The pooled samples were centrifuged at 5000 rpm for 15 mM in a Sigma 4K15C centrifuge (Sigma Laborzentrifugen GmbH, 37520, Germany), and the supernatant were collected and used to develop a reduction activity assay. The pooled samples contained both enzymes and substrates. The reactions of the enzymes and substrates generate NADH, which reduces resazurin or tetrazolium salts in the presence of other electron carriers or enzymes. For instance, the pooled samples were analyzed for activities that reduced resazurin to resorufin. The pooled lavage samples were added to wells of a 96-well plate in an amount of 50, 25, 12.5, 6.25 and 3.13 μl in duplicate. And then a 10 μl of reaction mix was added to each well. The volume in all wells was adjusted to 100 μl with 100 mM potassium phosphate of pH 7.5. The reaction mix contained 500 μM resazurin, 40 μM rotenone, 700 μM NAD+, 10 mM MgCl, and 100 mM potassium phosphate of pH 7.5. The reaction plate was carried out at room temperature, and covered with sealing film (Platemax AxySeal Sealing film, Axygen, Union City, Calif.) to prevent evaporation of reaction mixture. The fluorescence was measured every 5 min for 18 hours at Excitation 544/Emission 590 nm in a spectrometry plate reader (Spectra Max M3, Molecular Devices, Sunnyvale, Calif.). The results are shown in FIGS. 9A and 9B. Relative fluorescence unit (RFU) was calculated by dividing each fluorescence reading with that of the control wells, which did not contain any pooled oral lavage samples. The RFU numbers were correlated well with the amount of pooled lavage samples. The more pooled lavage samples, the higher the RFU number.

The fluorescence absorbance was also plotted, as shown in FIG. 9B. Again, the fluorescence absorbance was related to the amount of pooled oral lavage in the wells. The higher absorbance, the more pooled oral lavage samples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for reducing a tetrazolium salt comprising:
providing an oral cavity sample;
combining the oral cavity sample with a tetrazolium salt;
wherein the oral cavity sample comprises an enzyme and at least one of a dehydrogenase, reductase or reducing reagent; and
wherein the tetrazolium salt is reduced to produce a formazan dye.

2. The method of claim 1, wherein a biomarker is extracted from oral lavage, gingival brush samples and supragingival and subgingival plaques.

3. The method of claim 2, wherein the biomarker is extracted using, sonication, vortex and centrifugation.

4. The method of claim 2, wherein the extracted biomarker is analyzed with at least one of immunoassay, gradient hydrophilic interaction liquid chromatography with tandem mass spectrometry (HILIC/MS/MS), enzymatic assay, or colorimetric assay to quantify the levels of at least one of protein or enzyme.

5. The method of claim 2, wherein the biomarker is a protein.

6. The method of claim 5, wherein the protein is involved in glycolysis or cellular respiration pathway.

7. The method of claim 5, wherein the protein is at least one of: aldolase, triosephosphate isomerase, glyceraldehyde phosphate dehydrogenase, phosphoglycerate kinase, phosphoglycerate mutase, enolase, pyruvate dehydrogenase, lactate dehydrogenase, alcohol dehydrogenase, aconitase, isocitrate dehydrogenase, α-ketoglutarate dehydrogenase, succinyl coenzyme A synthetase, succinate dehydrogenase, fumarase, or malate dehydrogenase.

8. The method of claim 2, wherein the biomarker is a metabolite.

9. The method of claim 1, wherein the oral cavity sample comprises at least one of oral lavage sample, gingival brush sample, or gingival plaque sample.

10. The method of claim 1 wherein the oral cavity sample comprises a substrate.

11. The method of claim 1, wherein the oral cavity sample comprises an electron coupling reagent.

12. The method of claim 11 wherein the electron coupling reagent is at least one of diaphorase, 1-Methoxy-5-methylphenazinium methyl sulfate, 5-Methylphenazinium methyl sulfate, or Phenazine ethosulfate.

13. The method of claim 1, wherein the tetrazolium salt is at least one of MTT, EZMTT, MTS, XTT, INT, Nitro-TB, WST-1, WST-4, WST-5, WST-8, or WST-9.

14. The method of claim 1, wherein the oral cavity sample comprises a cofactor.

15. The method of claim 14, wherein cofactor is at least one of NAD+, NADP+, NADH, or NADPH.

16. A method for reducing resazurin comprising:
providing an oral cavity sample;
combining the oral cavity sample with resazurin;
wherein the oral cavity sample comprises an enzyme and at least one of a dehydrogenase, reductase or reducing reagent; and
wherein the resazurin is reduced to produce resorufin.
